(19) United States
(12) Patent Application Publication
(10) Pub. No.: US 2025/0101496 A1
(43) Pub. Date: Mar. 27, 2025

(54) METHODS FOR BALANCING ENCODING SIGNALS OF ANALYTES

(71) Applicant: Encodia, Inc., San Diego, CA (US)

(72) Inventors: Devin SULLIVAN, San Diego, CA (US); Norihito MURANAKA, San Diego, CA (US); Mark S. CHEE, Encinitas, CA (US)

(21) Appl. No.: 18/715,001

(22) PCT Filed: Dec. 21, 2022
(86) PCT No.: PCT/US2022/082187

§ 371 Date: May 30, 2024

Related U.S. Application Data
(60) Provisional application No. 63/292,406, filed on Dec. 21, 2021.

(51) Int. Cl. C12Q 1/6813; C12Q 1/6806; G01N 33/543; G01N 33/68

(52) U.S. Cl. CPC C12Q 1/6813; C12Q 1/6806; G01N 33/54366; G01N 33/68; G01N 2333/922; G01N 2440/40; G01N 2458/10

(57) ABSTRACT

The present disclosure relates to methods for high throughput analysis of analytes such as polypeptides in a cyclic manner (e.g., using NGPA or NGPS described herein) that allow adjustment of the dynamic range and sensitivity of analyte detection, for instance, based on the abundances of different polypeptides present in a sample to be analyzed. The approaches proposed herein can be used to adjust the dynamic range of abundant analytes (e.g., polypeptides present in high concentrations) in biological samples, such as plasma samples, and to increase proteome coverage achieved by high throughput protein analysis methods, for instance, by improving the sensitivity of detecting less abundant analytes.

### CROSS-REFERENCE TO RELATED APPLICATION

This application claims priority to U.S. Provisional Patent Application No. 63/292,406 filed Dec. 21, 2021, entitled “METHODS FOR BALANCING ENCODING SIGNALS OF POLYPEPTIDE ANALYTES,” which is herein incorporated by reference in its entirety for all purposes.

### REFERENCE TO AN ELECTRONIC SEQUENCE LISTING

The contents of the electronic sequence listing (776532003940SEQLIST.xml; Size: 31,811 bytes; and Date of Creation: Dec. 21, 2022) is herein incorporated by reference in its entirety.

### TECHNICAL FIELD

This disclosure generally relates to biotechnology, and in particular to analysis of immobilized analytes (e.g., polypeptides) in a cyclic manner employing binding of the analytes with binding agents conjugated with nucleic acid coding tags, followed by encoding of the binding events in nucleic acid libraries. The disclosure finds utility at least in a variety of methods and related kits for high-throughput analysis, including polypeptide identification or protein sequencing.

### BACKGROUND

Highly-parallel macromolecular characterization and recognition of proteins is challenging for several reasons. The use of affinity-based assays is often difficult due to several key challenges. One significant challenge is multiplexing the readout of a collection of affinity agents to a collection of cognate polypeptides; another challenge is minimizing cross-reactivity between the affinity agents and off-target polypeptides; a third challenge is developing an efficient high-throughput read out platform. An example of this problem occurs in proteomics in which one goal is to identify and quantitate most or all the proteins in a sample. Additionally, it is desirable to characterize various post-translational modifications (PTMs) on the proteins at a single molecule level. Currently this is a formidable task to accomplish in a high-throughput way. Improved methods and compositions for macromolecule analysis, especially high-throughput assays, are needed. This disclosure addresses these and other needs.

### BRIEF SUMMARY

The summary is not intended to be used to limit the scope of the claimed subject matter. Other features, details, utilities, and advantages of the claimed subject matter will be apparent from the detailed description including those aspects disclosed in the accompanying drawings and in the appended claims.

Recently, methods for high-throughput polypeptide characterization have been proposed, e.g., in US 2019/0145982 A1, US 2020/0348308 A1, US 2020/0348307 A1, US 2021/0208150 A1. US 2021/0396762 A1, WO 2022/040098 A1, and U.S. patent application Ser. No. 17/432,475 filed on Aug. 19, 2021, that utilize use of nucleic acid-encoded binding agents recognizing particular components of an immobilized polypeptide in a cyclic manner and encoding of binding agent binding history after each binding cycle in a nucleic acid recording tag, thus generating an extended recording tag. After encoding of information regarding binding agents that are bound to a plurality of immobilized polypeptides, the recording tags can be analyzed in parallel by, for example, next-generation sequencing (NGS), and information regarding structures of the polypeptides can be elucidated by decoding the information regarding binding agents that are bound to these polypeptides during each binding cycle.

One known problem that exists during analysis of biological samples, such as for example plasma samples, is the wide range of concentration of different proteins (dynamic range of plasma samples being estimated to be about 12 orders of magnitude between the lowest and the highest abundance proteins). Previous approaches include depletion of abundant proteins before sample preparation for the high-throughput assay (e.g., Kaur G, et al., Extending the Depth of Human Plasma Proteome Coverage Using Simple Fractionation Techniques. J Proteome Res. 2021 Feb. 5; 20 (2): 1261-1279), and commercial kits for removal of abundant proteins are available. However, existing solutions show high variability in performance. Accordingly, in order to perform high-throughput polypeptide characterization of biological samples, there remains a need for improved techniques relating to balancing concentrations of polypeptides before or during the polypeptide analysis. The disclosed methods allow for highly-parallelized, accurate and sensitive polypeptide characterization.

These and other aspects of the disclosure will be apparent upon reference to the following detailed description. To this end, various references are set forth herein which describe in more detail certain background information, procedures, compounds and/or compositions, and are each hereby incorporated by reference in their entireties.

Several variants of the ProteoCode™ assay that allow for high-throughput polypeptide characterization have been disclosed in US 2019/0145982 A1, US 2020/0348308 A1, US 2020/0348307 A1, US 2021/0396762 A1, WO 2022/040098 A1 and US 2021/0208150 A1. During an exemplary assay, an immobilized polypeptide associated with a nucleic acid recording tag is contacted sequentially with binding agents capable of binding to the polypeptide, wherein each binding agent comprises or is associated with a nucleic acid coding tag with identifying information regarding the binding agent. During each binding cycle, the coding tag and the recording tag are located in a sufficient proximity for interaction, and following the binding of the binding agent to the immobilized polypeptide, the identifying information regarding the binding agent bound to the polypeptide at this cycle is transferred from the coding tag to the recording tag by primer extension and/or ligation (chemical or enzymatic), e.g., primer extension followed by ligation, or ligation followed by primer extension, thus generating an extended recording tag. The binding cycle can be repeated multiple times using different binding agents interacting with the polypeptide, either separately, or in a mixture, and after completion of the binding cycles, binding history of the polypeptide is recorded in the extended recording tag.

High dynamic range in concentrations of polypeptide analytes present in a sample or biological samples represents a significant challenge for performing high-throughput polypeptide characterization with sufficient proteome coverage. Here, a few embodiments for adjusting signals generated from abundant (or known or suspected to be abundant) polypeptides (e.g., peptide chains, proteins, or protein complexes) during encoding assays are disclosed.

Provided herein is a method for analyzing a plurality of different polypeptides immobilized on a support, the method comprising:
(a) contacting the plurality of different polypeptides comprising a first polypeptide and a second polypeptide with a binder, wherein the first polypeptide is associated with a first recording tag and the second polypeptide is associated with a second recording tag, and wherein the binder comprises: (i) a binding moiety capable of binding to the first polypeptide; and (ii) a handle attached to the binding moiety and configured to bind to or react with the first recording tag;
(b) allowing the handle to bind to or react with the first recording tag brought in proximity by binding of the binder to the first polypeptide, thereby modifying the first recording tag to generate a modified first recording tag associated with the first polypeptide;
(c) optionally, fragmenting the plurality of different polypeptides immobilized on the support to generate fragments of different polypeptides immobilized on the support;
(d) contacting the plurality of different polypeptides or the fragments of different polypeptides with a plurality of binding agents, wherein each binding agent comprises: (i) a binding moiety capable of binding to a portion or component of a polypeptide of the plurality of different polypeptides or the fragments thereof; and (ii) a coding tag that comprises identifying information regarding the binding agent;
(e) allowing transfer of identifying information from coding tags of the plurality of binding agents to recording tags associated with the plurality of different polypeptides or the fragments of different polypeptides, thereby generating an extended second recording tag associated with the second polypeptide or fragment thereof upon binding of a binding agent to the second polypeptide or fragment thereof, wherein transfer of identifying information to the modified first recording tag associated with the first polypeptide or fragment thereof is suppressed or blocked; and
(f) analyzing the extended second recording tag to obtain identifying information regarding the binding agent that binds to the second polypeptide or fragment thereof, thereby obtaining information about the second polypeptide or fragment thereof, wherein the analyzing comprises nucleic acid sequencing.

Provided herein is a method for analyzing molecules of a polypeptide immobilized on a support, the method comprising:
(a) contacting the molecules of the polypeptide with a first binding agent and a second binding agent,
wherein each molecule of the polypeptide is associated with a recording tag immobilized on a support,
wherein the first binding agent comprises (i) a first binding moiety capable of binding to the polypeptide; and (ii) a first coding tag attached to the first binding moiety and comprising identifying information regarding the first binding agent, and
wherein the second binding agent comprises (i) a second binding moiety capable of binding to the polypeptide; and, optionally, (ii) a handle attached to the second binding moiety and configured to bind to or react with the recording tag;
(b) allowing transfer of the identifying information regarding the first binding agent from the first coding tag to the recording tag by primer extension and/or ligation to generate an extended recording tag, and optionally, allowing the handle to bind to or react with the first recording tag to generate a modified first recording tag;
(c) contacting the molecules of the polypeptide with a third binding agent comprising (i) a third binding moiety capable of binding to the polypeptide; and (ii) a third coding tag attached to the third binding moiety and comprising identifying information regarding the third binding agent;
(d) allowing transfer of the identifying information regarding the third binding agent from the third coding tag to the extended recording tag by primer extension and/or ligation to generate a further extended recording tag, wherein transfer of the identifying information regarding the third binding agent from the third coding tag to the recording tag or to the modified first recording tag is suppressed or blocked; and
(e) analyzing the further extended recording tag to obtain identifying information regarding the first binding agent and/or the third binding agent, thereby obtaining information about the polypeptide, wherein the analyzing comprises nucleic acid sequencing.

Reducing dynamic range of protein(s)/peptide(s) present in a sample before analysis by one of the high throughput peptide analysis improves the ability to detect low abundance protein(s)/peptide(s). Further, maintaining knowledge of input abundances improves quantification accuracy for peptide analytes.

### BRIEF DESCRIPTION OF THE DRAWINGS

Non-limiting embodiments of the present disclosure will be described by way of example with reference to the accompanying figures, which are schematic and are not intended to be drawn to scale. For purposes of illustration, not every component is labeled in every figure, nor is every component of each embodiment of the disclosure shown where illustration is not necessary to allow those of ordinary skill in the art to understand the disclosure.

FIGS. 1A-1C show exemplary variants of an encoding reaction involving information transfer, which forms a basis for macromolecule analysis. FIG. 1A depicts an exemplary assay using one or more binding agents that comprise (i) binding moieties specific for particular component(s) of a polypeptide attached to a support (e.g., a bead or a sequencing substrate such as a flowcell) and (ii) coding tag(s) with identifying information regarding the binding agent(s) (e.g., a barcode) and an optional UMI. Step 1 comprises immobilizing a polypeptide with an associated recording tag that comprises a barcode (e.g., a sample barcode, a fraction barcode, spatial barcode, a compartment tag, etc.) and an optional UMI (not shown). Step 2 comprises binding of a binding agent (e.g., the binding moiety being an antibody, Ab) conjugated with a coding tag that comprises a barcode with identifying information regarding the corresponding binding moiety (Ab1, Ab2, etc). Step 3 comprises transferring the identifying information from the coding tag to the recording tag, creating an extended recording tag (encoding step). Steps 2-3 form an encoding cycle during which the identifying information of the binding agent is encoded in the polypeptide-associated extended recording tag. Several encoding cycles can be employed during the assay (e.g., through cyclic binding of cognate binding agents to the immobilized polypeptide and corresponding information transfer after binding). After a series of sequential binding and coding tag information transfer steps, the final extended recording tag is produced, containing binding agent coding tag information including barcodes from “n” binding cycles providing identifying information for the binding moieties in the binding agents (e.g., antibody 1 (Ab1), antibody 2 (Ab2), antibody 3 (Ab3), . . . antibody “n” (Abn)), a barcode sequence from the recording tag, and flanking universal priming sequences at each end of the library construct to facilitate amplification and analysis by next-generation sequencing (NGS), such as digital NGS (dNGS).

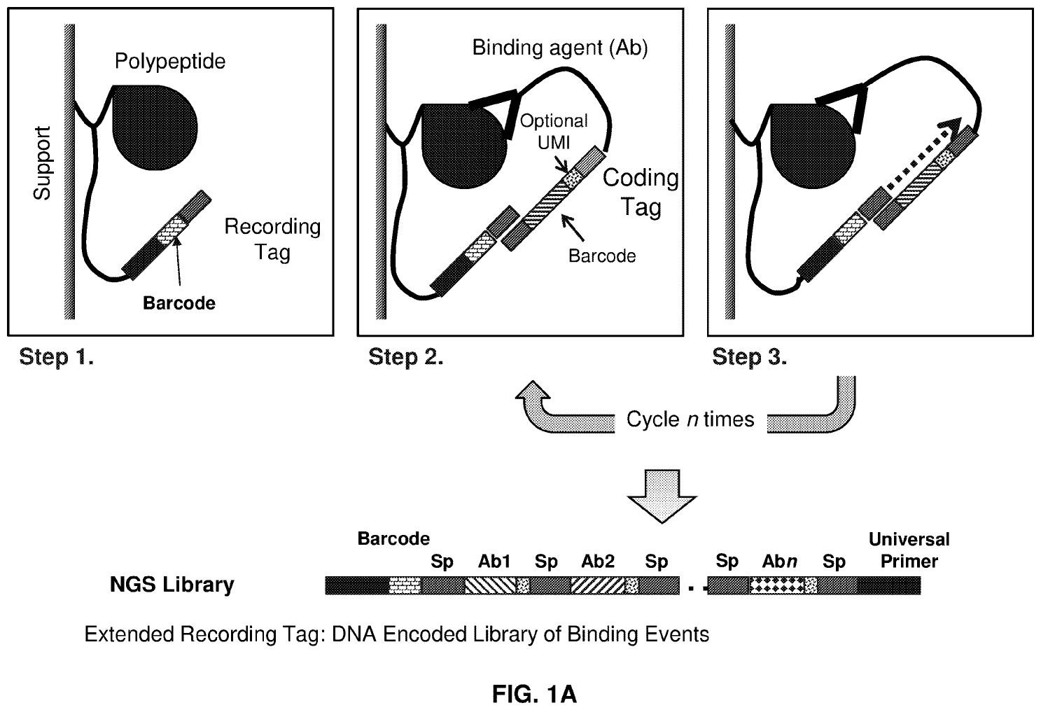

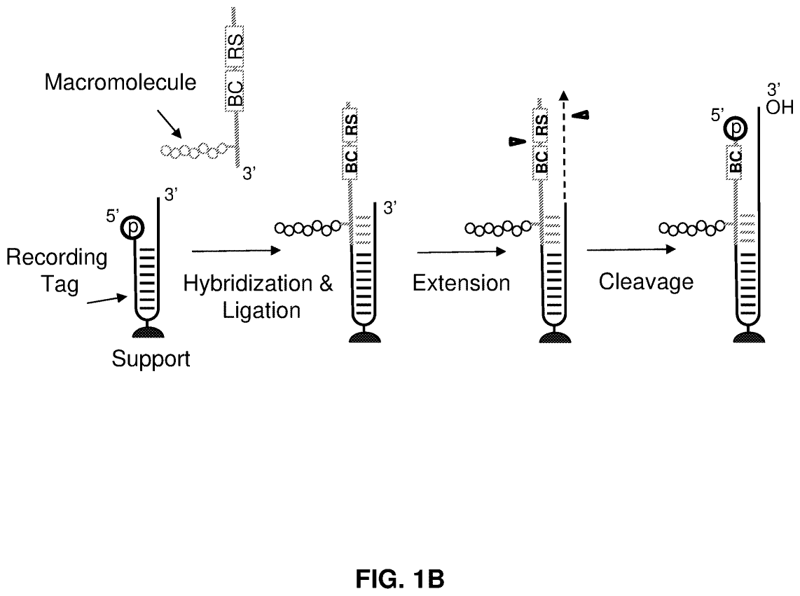

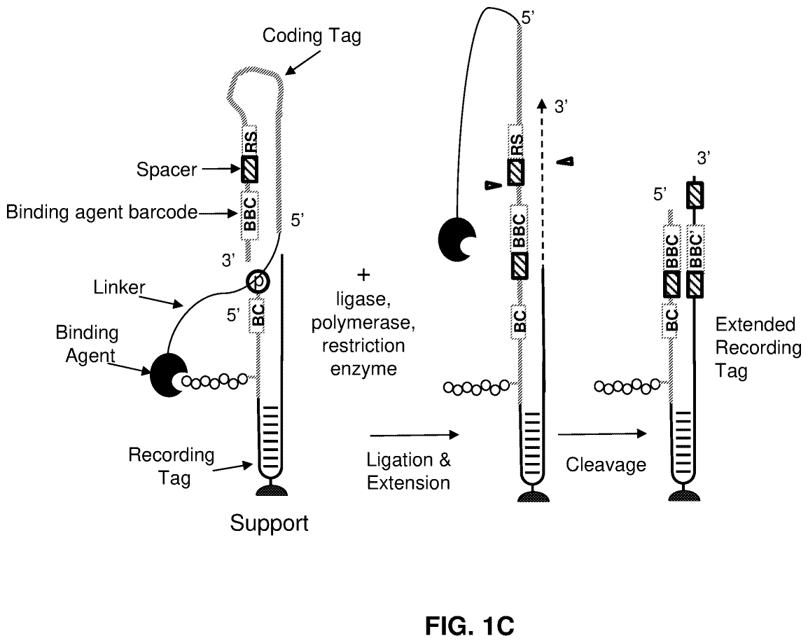

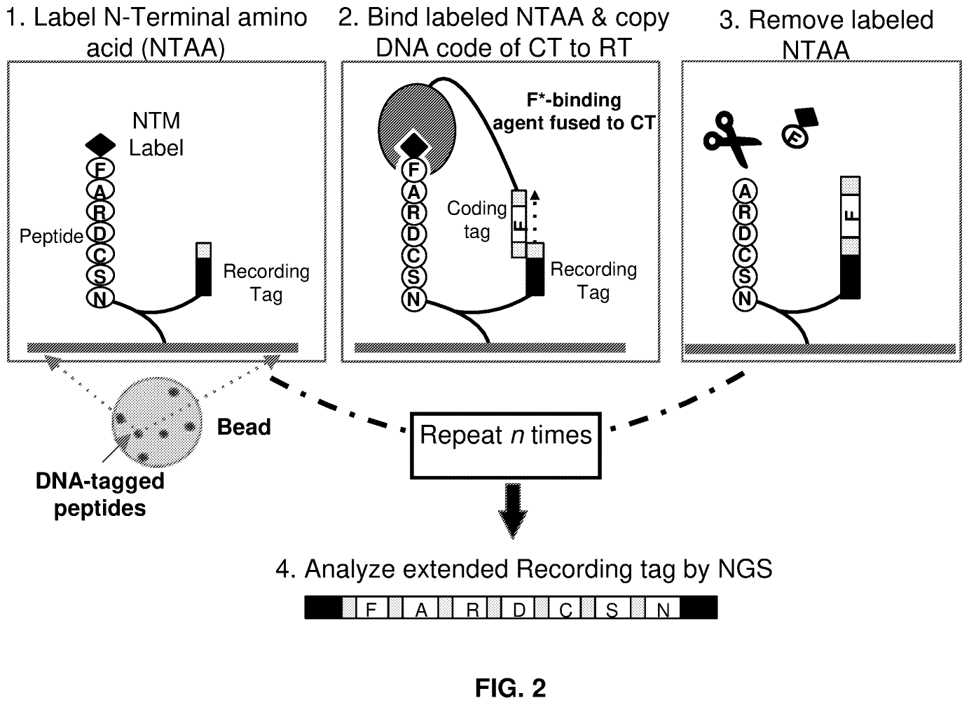

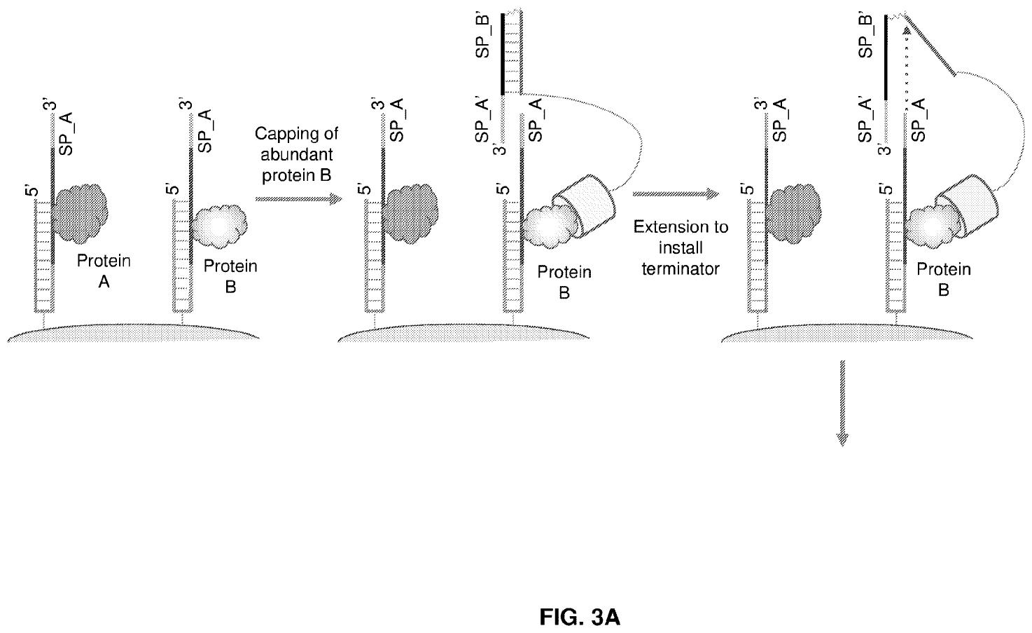

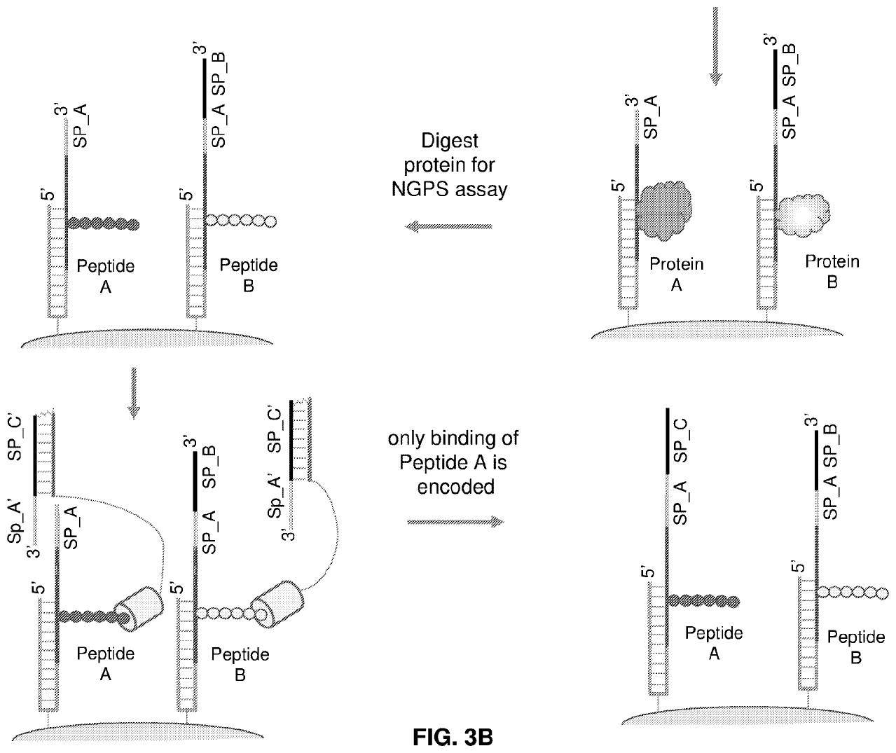

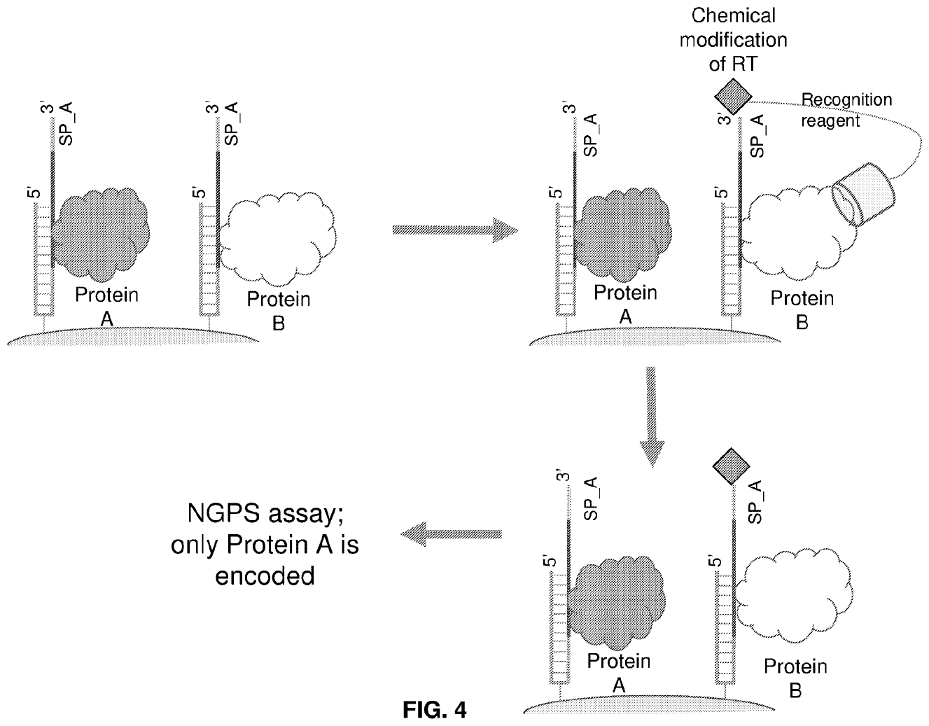

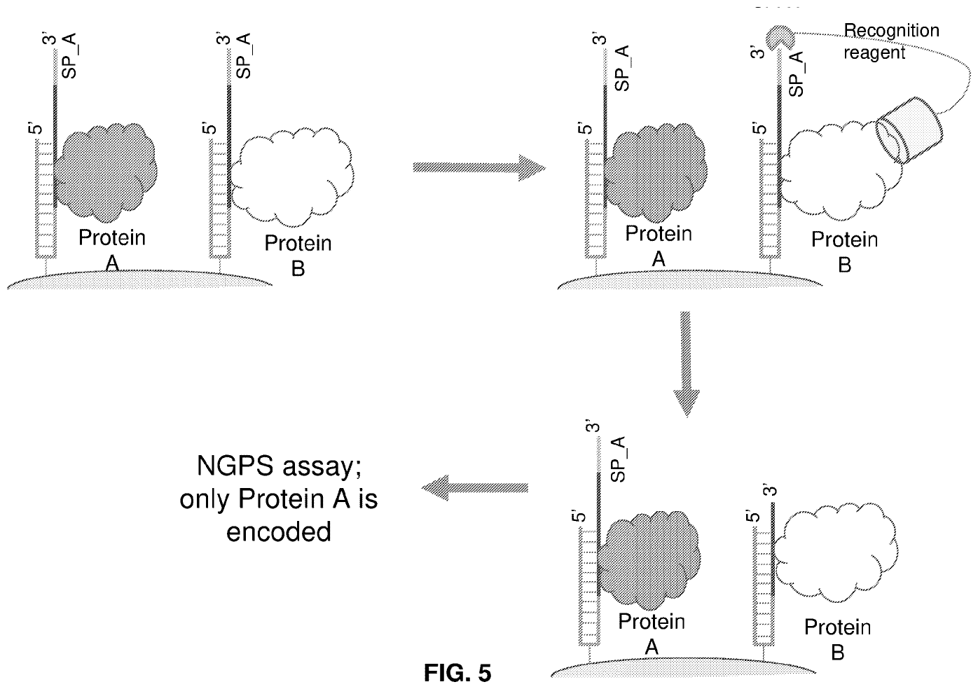

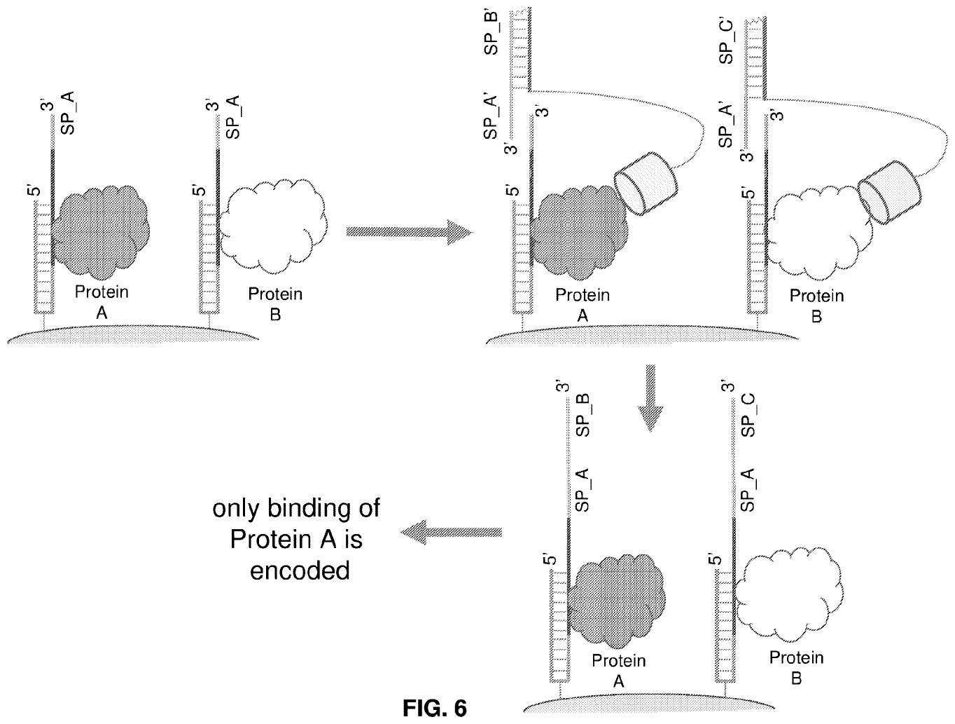

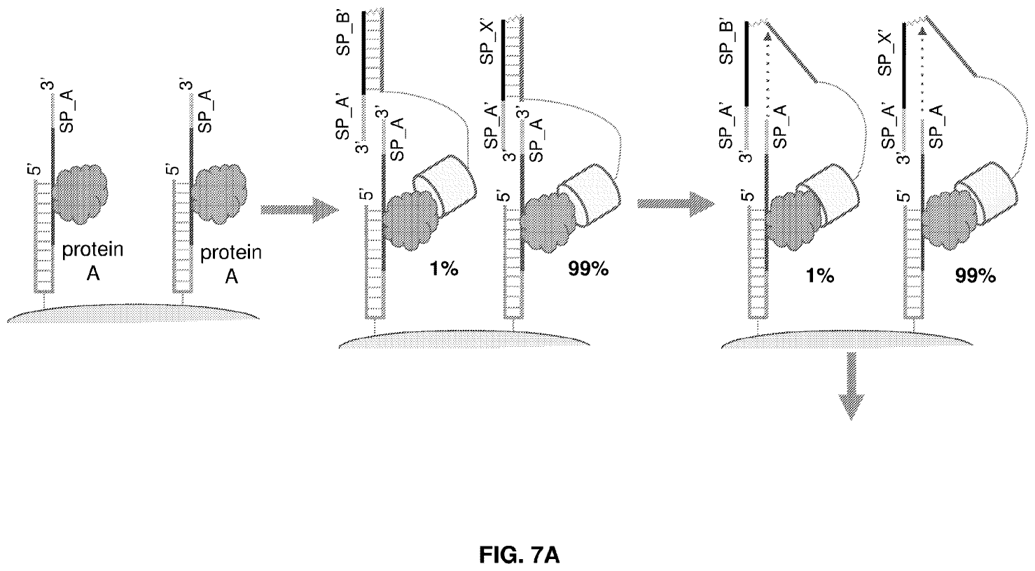

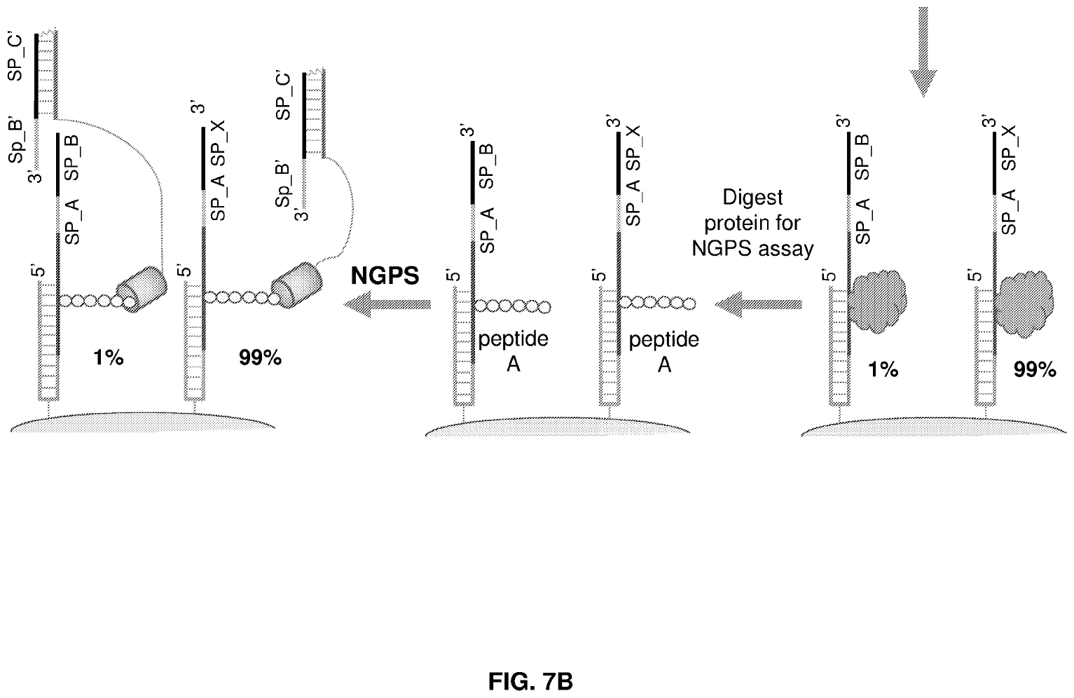

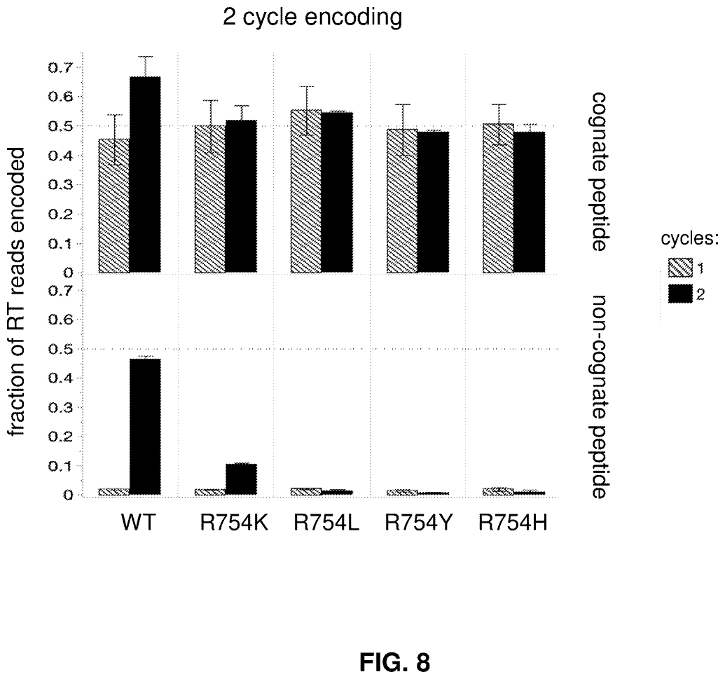

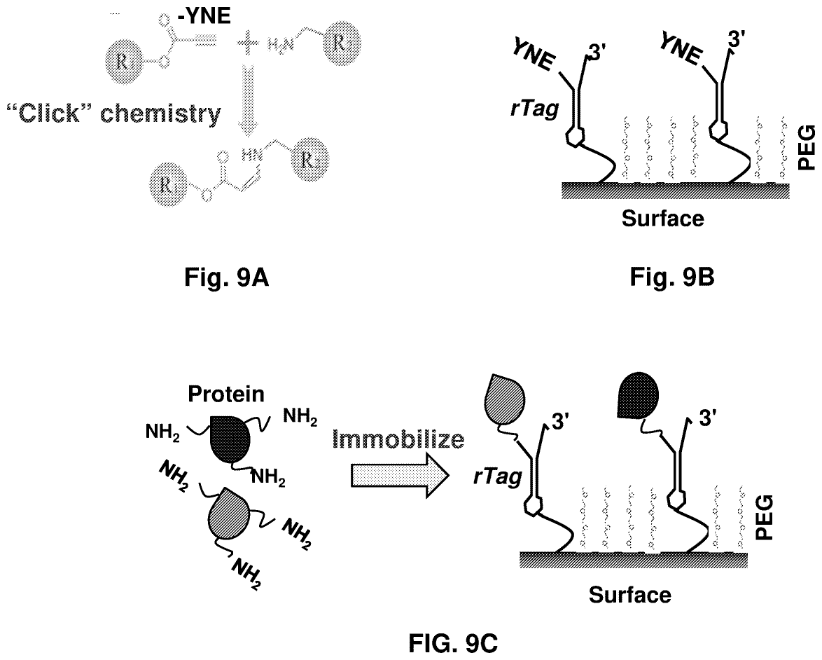

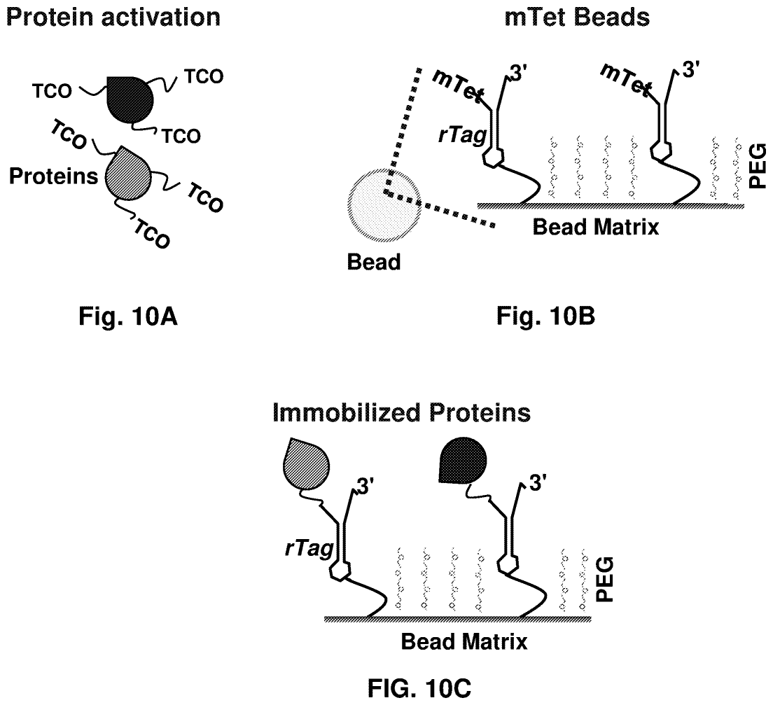

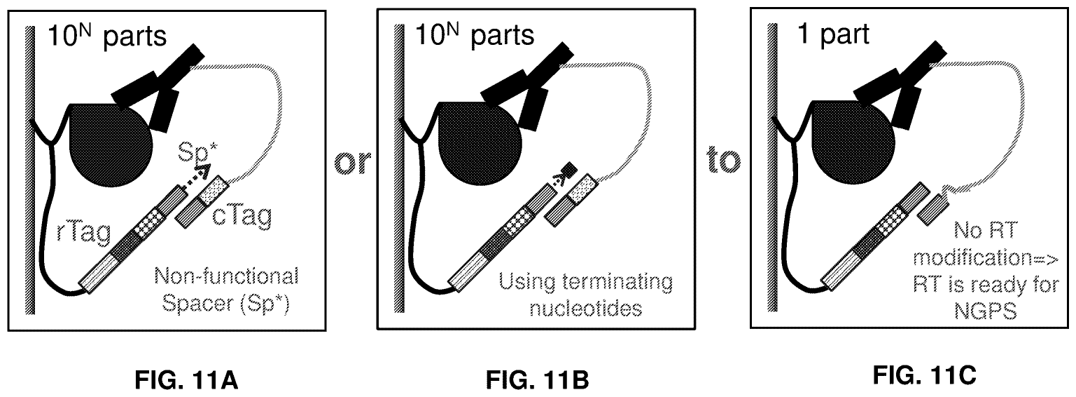

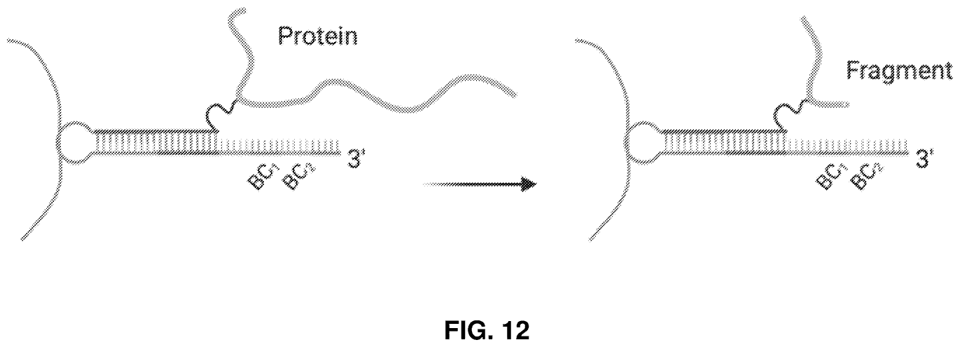

In FIG. 1B, a macromolecule (e.g., a peptide) to be analyzed is joined to a recording tag hairpin immobilized on a support using a ligation reaction and the structure is cleaved for subsequent steps. First (leftmost) panel of FIG. 1B shows a capture nucleic acid hairpin with recessed 5′ phosphorylated end. Second panel of FIG. 1B shows a peptide to be analyzed is attached to a bait nucleic acid which hybridizes to the capture nucleic acid hairpin immobilized on a support (e.g., via a reactive coupling moiety). The bait nucleic acid is ligated to the capture nucleic acid. The recording tag is ligated to the hairpin. Block-labeled barcode (BC) indicates any optional barcodes, e.g. sample-specific barcode &/or UMI that can be attached to the macromolecule and incorporated to the recording tag. Block-labeled restriction enzyme recognition site (RS) site represents an incorporated sequence for a type IIS restriction enzyme to recognize and cleave. Third panel of FIG. 1B shows polymerase extension to produce a double stranded DNA (dsDNA) construct with the peptide attached. Fourth panel of FIG. 1B shows the dsDNA construct following digestion with a type IIS RE to produce a 3′ overhang (e.g., 2-base pair sequence) with a recessed 5′ phosphorylated end. In this manner, the recording tag containing one or more barcodes is prepared and available for information transfer from a coding tag.

FIG. 1C shows a cycle of encoding with the structure generated in FIG. 1B. The left panel of FIG. 1C shows a binding agent bound to the peptide, bringing a coding tag attached to the binding agent into proximity with the recording tag. In some embodiments, the binding agent can be attached or joined to the coding tag in locations other than depicted (e.g., at the loop region of the coding tag or others). The binding agent as shown is attached to coding tag by a linker. The coding tag contains a binding agent-specific barcode (BBC), a 2 bp spacer, and a type IIS restriction enzyme site (RS). The middle panel of FIG. 1C shows the product of first two enzymatic reactions. Upon ligation of the 5′ end of the recording tag to the 3′ end of the coding tag, polymerase extends the 3′ (non-ligated) end of the recording tag to create a dsDNA molecule containing 2-base pair spacers adjacent to their respective type IIS RE sites. Following double stranding, the type IIS RE binds and cuts adjacent to its recognition site. The right panel of FIG. 1C illustrates the final product after all 3 enzymatic steps, where the dsDNA now contains the binding agent-specific barcode and a 3′ overhang (OH), e.g., a 2 nt overhang, which serves as the spacer sequence. In some embodiments, after a cycle of information transfer, a portion of the polypeptide for analysis can be removed from the polypeptide. The cycle of steps shown in FIG. 1C may be repeated one or more times with additional binding agents and coding tags to further extend the recording tag.

The exemplary assays described in FIG. 1A and FIG. 1C can be referred to as next generation protein assay (NGPA).

FIG. 2 depicts an exemplary polypeptide sequencing assay with N-terminal amino acid (NTAA)-specific binding agents. (1) Peptide molecules are each associated with a recording tag (RT) (e.g., a DNA) and attached to beads at a low peptide/RT pair (e.g., a peptide/RT conjugate) density, a sparsity that permits only intra-peptide/RT pair information transfer to occur. For instance, the peptide and its RT can form a construct, and in cases where the peptide is covalently attached to the RT to form a single molecule, the construct sparsity on a bead permits only intramolecular information transfer, and not information transfer between adjacent constructs on the bead. The peptide N-terminal amino acid (NTAA) residues are labeled with an N-terminal modification (NTM). (2) Next, immobilized and labeled peptides are contacted with binding agents specific for labeled NTAA (labeled F-specific binding agent is shown). Each binding agent comprises a coding tag (CT) (e.g., DNA) that comprises identifying information regarding the binding moiety in the binding agent. After binding and washing, the coding tag identifying information is transferred enzymatically to the recording tag (via extension and/or ligation, such as primer extension followed by ligation), generating an extended RT. (3) The labeled NTAA is removed, e.g., by using mild Edman-like elimination chemistry or by a Cleavase enzyme. The cycle 1-2-3 is repeated n times. After n cycles, the extended RT representing the n amino acids of the peptide sequence is formed and can be sequenced by NGS. A representative structure of the extended RT after 7 cycles is shown. The exemplary assay described in FIG. 2 can be referred to as next generation peptide sequencing (NGPS).

FIGS. 3A-3B show exemplary capping of abundant protein B prior to a polypeptide sequencing assay (e.g., as shown in FIG. 2). A plurality of proteins including protein A and protein B are immobilized on a support (e.g., a bead) and each protein is associated with a recording tag. Abundant protein B is targeted with a binding agent comprising a coding tag that upon binding to protein B modifies the recording tag associated with protein B. Next, the plurality of proteins was digested and peptides that remain immobilized on the solid support are subjected to the polypeptide sequencing assay. Protein A associated with the unmodified recording tag is encoded, and encoding (e.g., transfer of identifying information regarding binding agent) of protein B is blocked.

FIG. 4 shows an exemplary embodiment of capping of abundant protein B prior to a polypeptide sequencing assay. A plurality of proteins including protein A and protein B are immobilized on a support (e.g., a bead) and each protein is associated with a recording tag. Abundant protein B is targeted with a binding agent comprising a chemical moiety that upon binding to protein B modifies the recording tag associated with protein B. Next, the plurality of proteins is subjected to the polypeptide sequencing assay. Protein A associated with the unmodified recording tag is encoded, and encoding of protein B is blocked.

FIG. 5 shows an exemplary embodiment of capping of abundant protein B prior to a polypeptide sequencing assay. A plurality of proteins including protein A and protein B are immobilized on a support (e.g., a bead) and each protein is associated with a recording tag. Abundant protein B is targeted with a binding agent conjugated to an enzyme (e.g., a nuclease) that upon binding to protein B modifies (e.g., cleaves) the recording tag associated with protein B. Next, the plurality of proteins is subjected to the polypeptide sequencing assay. Protein A associated with the unmodified recording tag is encoded, and encoding of protein B is blocked.

FIG. 6 shows an exemplary embodiment of capping of abundant protein B prior to a protein assay. A plurality of proteins including protein A and protein B are immobilized on a support (e.g., a bead) and each protein is associated with a recording tag. Prior to the protein assay, protein A and protein B are contacted with binding agents specific for protein A and for protein B, respectively, and conjugated to coding tags comprising either SP_B′ or SP_C′, which are different spacer sequences. Following binding of the binding agents to protein A and protein B, the recording tags associated with protein A and protein B are extended (modified) to contain either SP_B or SP_C spacer sequences. Next, the plurality of proteins is subjected to the protein assay. Protein A associated with the recording tag having the SP_B sequence can be encoded by using binding agents comprising the SP_B′ sequence, and encoding of the recording tag having the SP_C sequence is blocked since it cannot hybridize to the SP_B′ sequence to initiate information transfer for encoding.

FIGS. 7A-7B show an exemplary competitive attenuation of encoding during a polypeptide sequencing assay for an abundant protein A. A plurality of proteins including protein A is immobilized on a support (e.g., a bead) and each protein is associated with a recording tag. Prior to polypeptide sequencing, protein A is contacted with a mixture of binding agents specific for Protein A and conjugated with coding tag comprising either SP_B (e.g., 1% of binding agents) or SP_X (e.g., 99% of binding agents). Following binding of the binding agents to protein A, the recording tags associated with Protein A are extended (modified) to contain either SP_B′ or SP_X′, which are different spacer sequences. Next, the plurality of proteins is subjected to the polypeptide sequencing assay, for instance by digesting protein A into peptides followed by NGPS assay. Peptides from protein A that remain attached to the support and are associated with the recording tag having the SP_B′ sequence can be encoded (which comprises further extending associated recording tags) by using binding agents comprising the SP_B′ sequence, and encoding of the recording tag having the SP_X′ sequence is blocked since it cannot hybridize to the SP_B′ sequence to initiate information transfer for encoding.

FIG. 8. Exemplary multicycle encoding assay for Klenow Fragment (KF) exo-WT enzyme and its mutants. See Example 5 for details. The encoding yields were shown as fractions of recording tag (RT) reads encoded. Results of two cycles encoding are shown, and similar results for 5 and more cycles of encoding can be obtained by the same methods. The KF WT enzyme produces a high (about 50% relative to the specific signal) non-specific signal for the 2nd cycle of the encoding on the non-cognate peptide, while the mutants produce much lower non-specific signal for the 2nd cycle of the encoding.

FIGS. 9A-9C. Exemplary immobilization of native proteins on beads via a single attachment point and a single associated recording tag. FIG. 9A shows amino-yne “click” chemistry is used to immobilized proteins by native amines to an activated bead surface. FIG. 9B shows beads are designed with hairpin recording tags (rTags) containing a 5′ “yne-click” moiety at the 5′ end and a 3′ extendable primer. The lateral density of the rTags is controlled by titrating a bead conjugation moiety with capped methyl-PEG moieties which also provide bead passivation. After protein attachment in FIG. 9C, barcodes such as sample barcodes can be incorporated into the hairpin sequence.

FIGS. 10A-10C. Exemplary immobilization of derivatized proteins on beads via a single attachment point and a single associated recording tag. As an alternative to the native immobilization chemistry, alternative “click chemistry” modalities such as iEDDA click pairs can be employed. FIG. 10A shows the proteins can be first derivatized at native lysine amines using an NHS-PEG-TCO reagent, and FIG. 10B shows the beads can be derivatized with mTet near the 5′ terminus of the hairpin. FIG. 10C shows incubation of the TCO-derivatized proteins with the mTet-derivatized beads generates a single point attachment (given sufficient lateral spacing distance) at the site of an attached recording tag.

FIGS. 11A-11C. Exemplary strategies for suppression of recording tags (rTags) associated with abundant proteins via “terminating” coding tag (cTag) antibody-based methods. FIG. 11A shows an antibody to an abundant protein comprises a “terminating” cTag, which drives primer extension of the rTag associated with the abundant protein creating a non-functional spacer element Sp*. The spacer element is unable to participate in further primer extension reactions during the next rounds of protein analysis (e.g., during NGPS assay). FIG. 11B shows an antibody to an abundant protein comprises a “terminating” cTag which drives primer extension of rTag in the presence of a terminating nucleotide, such that the extended rTag is blocked from further primer extension by a terminating nucleotide. The terminating nucleotide may be an irreversible terminator (e.g., a ddNTP) or a reversible terminator, where the blocking group(s) in the reversible terminator can be controllably removed, allowing further primer extension. FIG. 11C shows the same antibody molecule as shown in FIG. 11A or FIG. 11B, but with no extension or termination moiety attached so that after a binding event, rTag associated with the protein remains unmodified (e.g., neither extended nor blocked from primer extension) and ready for the next rounds of protein analysis (e.g., NGPS assay). For instance, the linker between the antibody molecule and the spacer element in the cTag shown in FIG. 11C can be a non-nucleic acid linker, such that upon hybridization of the spacer elements in the cTag and the rTag, the linker cannot serve as a template for primer extension of the rTag. Variants of binders shown in FIG. 11A (binder A) or FIG. 11B (binder B) may be used in combination with binder shown in FIG. 11C (binder C), and the ratio of binder A or B to binder C may be adjusted to reflect the level of suppression needed. For example, when 10\ parts of binder A (or binder B) are mixed with one part of binder C, this ratio results in N logs suppression of rTag signal from the abundant protein during the next rounds of protein analysis.

FIG. 12. Exemplary conversion of protein assay format to polypeptide sequencing assay format. Immobilized protein (with associated recording tag) is denatured, for example, by exposure to sodium dodecyl sulphate (SDS). The denatured protein can be fragmented (e.g., using trypsin digestion), leaving only the fragment (e.g., a random tryptic peptide fragment) that is anchored to the bead by a single attachment point; the retained peptide fragment is sequenced by a polypeptide sequencing assay. For proteins of a biological sample, different tryptic fragments may be sequenced from different molecules of the same protein due to random immobilization of protein molecules via different single attachment points.

### DETAILED DESCRIPTION

High dynamic range in concentrations of polypeptide analytes present in biological samples represents a significant challenge for performing high-throughput polypeptide characterization, such as by performing the ProteoCode™ assay, due to non-sufficient representation of minor proteins present in a sample which results in low quality or absent information regarding them. Here, several embodiments for adjusting signals generated from known abundant polypeptides or polypeptides during the encoding assays are disclosed. In some embodiments, these approaches are designed to be used in combination with an NGPA (next generation protein assay) or NGPS (next generation peptide sequencing) assays (collectively called the ProteoCode™ assay) disclosed in the published US patent application US 2019/0145982 A1, see also FIG. 1A, FIG. 1C, and FIG. 2. The NGPS peptide sequencing assay comprises several chemical and enzymatic steps in a cyclical progression. The fact that NGPS sequencing is at a single molecule level confers several key advantages to the process, including robustness to inefficiencies in the various cyclical chemical/enzymatic steps.

A first exemplary NGPA/NGPS method for analyzing a macromolecule (e.g., polypeptide) analyte comprises the following steps:
(a) providing the polypeptide analyte and an associated recording tag joined to a solid support;
(b) contacting the polypeptide analyte with a first binding agent capable of binding to the polypeptide analyte, wherein the first binding agent comprises a first coding tag that comprises identifying information regarding the first binding agent;
(c) following binding of the first binding agent to the polypeptide analyte, transferring the identifying information regarding the first binding agent from the first coding tag to the recording tag to generate a first order extended recording tag;
(d) contacting the polypeptide analyte with a second binding agent capable of binding to the polypeptide analyte, wherein the second binding agent comprises a second coding tag that comprises identifying information regarding the second binding agent;
(c) following binding of the second binding agent to the polypeptide analyte, transferring the identifying information regarding the second binding agent from the second coding tag to the first order extended recording tag to generate a second order extended recording tag; and
(f) analyzing the second order extended recording tag, wherein analyzing comprises a sequencing method, and obtaining the identifying information regarding the first binding agent and the identifying information regarding the second binding agent to provide information regarding the polypeptide analyte, thereby analyzing the polypeptide analyte.

A second exemplary NGPA/NGPS method for analyzing a macromolecule (e.g., polypeptide) analyte comprises the following steps:
(a) providing the polypeptide analyte and an associated recording tag joined to a solid support;
(b) contacting the polypeptide analyte with a binding agent capable of binding to the polypeptide analyte, wherein the binding agent comprises or is configured to be associated with a coding tag that comprises identifying information regarding the binding agent, to allow binding between the polypeptide analyte and the binding agent;
(c) generating a double stranded extended recording tag by (i) joining at least one end of the recording tag to an end of the coding tag by a nucleic acid joining reagent, and (ii) optionally, extending the recording tag using the coding tag as a template by a polymerase; and
(d) cleaving the double stranded extended recording tag with a double strand nucleic acid cleaving reagent to generate a truncated extended recording tag;
whereby the identifying information regarding the binding agent is transferred from the coding tag to the recording tag to generate the double stranded extended recording tag, and is present in the truncated extended recording tag; and
(c) analyzing one or more of the truncated extended recording tags, wherein analyzing the extended recording tag(s) comprises a nucleic acid sequencing method.

In preferred embodiments of the second exemplary NGPA/NGPS method, in step (d) the double stranded extended recording tag comprises a recognition sequence capable of being recognized by the double strand nucleic acid cleaving reagent, and the cleavage of the double stranded extended recording tag releases the binding agent from the polypeptide analyte.

In preferred embodiments of the second exemplary NGPA/NGPS method, steps (b), (c) and (d) are repeated sequentially one or more times in a cyclic manner, and a 3′ overhang of the extended recording tag is generated by the double strand nucleic acid cleaving reagent in the cleavage step (d) that is available to hybridize with a second coding tag when the contacting step (b) is repeated.

In preferred embodiments of the NGPA methods, binding agent (e.g., an antibody or an aptamer) is configured to recognize a specific epitope on the immobilized polypeptide (FIG. 1A and FIG. 1C). Instead, in preferred embodiments of the NGPS methods, binding agent (e.g., an engineered protein or an aptamer) is configured to recognize an N-terminal amino acid (NTAA) or a modified (functionalized) NTAA on the immobilized polypeptide (NTAA-specific binding agents, FIG. 2). The steps of NGPS also include cleavage of the modified NTAA after binding and encoding steps. Then, the steps of NTAA functionalization, binding, encoding and cleavage are repeated n times to generate a DNA-encoded library on the recording tag associated with the immobilized peptide, representing identifying information at least for some amino acid residues of the immobilized peptide. Analysis by sequencing of the recording tag (or a complement thereof) after completion of the n cycles provides the identifying information for these amino acid residues (both identities and order of the amino acid residues can be decoded from the sequence of the recording tag), which results in identification of the immobilized peptide. In some embodiments, entire binding history of the immobilized peptide can be encoded in a nucleic acid recording tag associated with the immobilized peptide, and then decoded during analysis, revealing the peptide identity.

In some embodiments, both NGPA and NGPS can be combined in one assay for characterization and sequencing of the immobilized peptide. For example, at first, the NGPA assay utilizing a PTM-specific antibody is employed, which recognizes the corresponding PTM (post-translational modification) on the immobilized peptide, and encodes this information on the recording tag. Next, the NGPS assay is employed on the same immobilized peptide (or the immobilized peptide further digested with a protease, such as trypsin, to generate a smaller immobilized peptide associated with the same recording tag). Thus, both information regarding specific PTM(s) and information regarding amino acid residues of the immobilized peptide is encoded in the same recording tag associated with the immobilized peptide. Typically, for successful encoding (which comprises transferring the identifying information regarding the binding agent bound to the peptide from the coding tag of the binding agent to the recording tag), binding agents have affinity (Kd) to a component of the polypeptide of less than 500 nM, and preferably less than 100 nM; more preferably in the range of 10-100 nM, and even more preferably in the range of 1-10 nM.

In some embodiments, a component of the polypeptide may be a single terminal amino acid residue of the polypeptide (e.g., an NTAA or a CTAA) or a single terminal amino acid residue of the polypeptide modified by a functionalizing reagent (e.g., a modified NTAA or a modified CTAA). In some embodiments, a component of the polypeptide comprises a single terminal amino acid residue of the polypeptide or a single terminal amino acid residue of the polypeptide modified by a functionalizing reagent. In some embodiments, a component of the polypeptide may be a terminal dipeptide, terminal tripeptide, or a terminal oligopeptide. In some embodiments, a component of the polypeptide may be a terminal dipeptide, terminal tripeptide, or a terminal oligopeptide modified by a functionalizing reagent. In some embodiments, a component of the polypeptide may be an internal residue, internal dipeptide, internal tripeptide or internal oligopeptide.

The described approach can be used to characterize and/or identify at least 100, at least 500, at least 1000, at least 5000, at least 10000, at least 100,000, or at least 1,000,000 of different polypeptide analytes simultaneously (i.e., in a single assay). However, overrepresentation of abundant proteins in biological samples can result in reduced proteome coverage, including missing information regarding polypeptides present in low concentrations. Thus, methods are needed that would decrease encoding from abundant polypeptides and/or increase encoding from low concentration polypeptides present in a sample. This problem is addressed or solved herein by the disclosed methods as provided below.

In various disclosed embodiments, a dynamic range modulation is achieved using NGPA-based attenuation of the encoding signal from abundant or selected polypeptide analytes prior to NGPS. In some exemplary embodiments, proteins from a sample (e.g., biological sample) are immobilized on solid support-attached activated recording tags via a single attachment point, preferably a lysine or derivatized lysine residue of proteins. The remaining lysine residues of the proteins are capped with a reactive capping agent. Immobilized proteins are optionally fragmented before or after immobilization by a protease, such as trypsin, LysC, proteinase K, etc., and some fragments of proteins are associated with a recording tag immobilized on a solid support. Herein, proteins and fragments of proteins are collectively called polypeptides. For analysis, selected immobilized polypeptides each associated with a recording tag are contacted with at least one binder, wherein the binder comprises: (i) a binding moiety capable of binding to a first polypeptide of the plurality of different polypeptides; and (ii) a handle attached to the binding moiety and configured to bind or react, when in proximity, to a first recording tag associated with the first polypeptide. Following binding of the binder to the first polypeptide, conditions are provided to allow the handle to bind to or react with the first recording tag, thereby modifying the first recording tag and generating a modified first recording tag. In some embodiments, the purpose of generating the modified first recording tag associated with the first polypeptide is to prevent this tag from being used in later rounds of binding and encoding (i.e., making a “dead end” first recording tag). Specific conditions for this modification depend on the structure of the handle, which may comprise a polynucleotide, a protein enzyme, or a small chemical moiety. In some embodiments, regardless of the structure of the handle, reaction between the handle and the first recording tag produces an unfunctional first recording tag associated with the first polypeptide. In preferred embodiments, such reaction is configured to prevent (block) further extension of first recording tag, effectively terminating further encoding reaction for selected polypeptide analytes (in this case, the first polypeptide). The binder in this example can be viewed as a “dead-end” binder in contract to “productive” binders. In addition to a binding moiety capable of binding to the first polypeptide, an “productive” binder also contains a coding tag that comprises identifying information regarding the binder, wherein the coding tag can extend the first recording tag during encoding reaction, and is configured to leave the first recording tag amenable to further extension reactions (e.g., following binding of additional binding agents to the first polypeptide).

In yet other embodiments, the handle comprises a coding tag comprising an encoder sequence comprising identifying information regarding the binder and/or a spacer sequence. In these embodiments, the modified first recording tag incorporates the encoder sequence and/or the spacer sequence, and can be further extended in next rounds of binding and encoding. These embodiments can be viewed as “positive capping”, and may be used for targeted analysis of selective analytes, wherein each analyte would be labeled with its own specific binder (such as specific antibody or aptamer).

In some embodiments, NGPA-based attenuation of the encoding signal from abundant or selected polypeptide analytes is achieved by contacting a first polypeptide with a set of binders, wherein the set contains x % “productive” binders and (100−x) % “dead-end” binders, effectively reducing the number of peptides derived from the first polypeptide that can be encoded in downstream NGPS assay by a factor of 100%/x %. In one embodiment, the set of binders contains 1% “productive” binders and 99% “dead-end” binders (e.g., as shown in FIG. 7A), effectively reducing the number of peptides derived from the first polypeptide that can be encoded in downstream NGPS assay by a factor of 100. In some embodiments, x % can be 0.01%, 0.1%, 0.5%, 1%, 5%, 10%, 50% or any other number from 0% to 100%, such as a number between any two of the aforementioned percentages. Any two or more binders of the set of binders can have the same binding moiety or different moieties that bind to the same region of the first polypeptide. For instance, a “productive” binder and a “dead-end” binder can have the same binding moiety. In other instances, a “productive” binder and a “dead-end” binder can have different binding moieties, which can bind to the same region of the first polypeptide or different regions of the first polypeptide.

In some embodiments, “dead-end” encoding tag is configured to have a different spacer element (or devoid of spacer element) than used in the NGPS assay. Thus, the modified first recording tag associated with the first polypeptide will contain an incompatible spacer element (or devoid of spacer element) and will not be extended in further extension reactions (e.g., following binding of additional binding agents to the first polypeptide). In other embodiments, prevention of further extension reactions occurs by modifying the first recording tag associated with the first polypeptide chemically or enzymatically, generating an unextendible first recording tag.

In some exemplary embodiments, immobilization of polypeptide analytes on a solid support occurs as follows. A protein mix (e.g., cell lysate) is incubated with a solid support (e.g., porous beads) containing spatially separated recording tags attached to a bioconjugatable moiety, wherein this moiety is configured to be conjugated to reactive amino acid residues, or derivatized residues, on protein analytes. The spatial separation and single recording tag stoichiometry ensures that a protein can be immobilized by a single attachment point (semi-random depending on the location of the reactive amino acid residues). Derivatized residues on protein analytes can be produced by a reactive probe configured to specifically react with an amino acid residue. A number of such probes is known in the art to target specific amino acid types, such as cysteines, lysines, tyrosines, aspartates and glutamates, methionines, tryptophans, histidines and arginines. For example, reactive alkyne probes have been developed to specifically target particular amino acid types in proteins and were evaluated on broad sets of protein targets (Zanon PRA, et al. Profiling the proteome-wide selectivity of diverse electrophiles. ChemRxiv. Cambridge: Cambridge Open Engage; 2021; Gehringer, M. & Laufer, S. A. Emerging and Re-Emerging Warheads for Targeted Covalent Inhibitors: Applications in Medicinal Chemistry and Chemical Biology. J. Med. Chem. 2019, 62, 5673-5724; Parker, C. G. & Pratt, M. R. Click Chemistry in Proteomic Investigations. Cell 2020, 180, 605-632). Some examples of such reactive alkyne probes include: IA-alkyne and EBX2-alkyne for cysteines; STP-alkyne, ArSq-alkyne and EBA-alkyne for lysines; PTAD-alkyne and SuTEx2-alkyne for tyrosines; HC-alkyne, MeTet-alkyne and Az-alkyne for aspartates and glutamates; OxMet2-alkyne for methionines; CP-alkyne, HMN-alkyne, MMP-alkyne for tryptophans; and CP-alkyne for histidines; PhGO-alkyne for arginines (see U.S. patent application Ser. No. 18/065,282 filed on Dec. 13, 2022, and Zanon PRA, et al. Profiling the proteome-wide selectivity of diverse electrophiles. ChemRxiv. Cambridge: Cambridge Open Engage; 2021). In one embodiment, a DNA tag having a reactive moiety is used to label the side chain amino groups on lysines of polypeptides using standard bioconjugation methods (see US20190145982 A1). Other examples include utilizing click chemistry reagents to target selected amino acid residues, such as lysines. N-hydroxysuccinimide (NHS) is an amine reactive coupling agent, and Dibenzocyclooctyl (DBCO) is a strained alkyne useful in “click” coupling to the surface of a solid support. In one embodiment, DNA tags are coupled to side chain amines of lysine (K) residues of protein analytes via NHS moieties. Also, a heterobifunctional linker, NHS-alkyne, can be used to label side chain amines of lysine residues to create an alkyne “click” moiety. Azide-labeled DNA tags can then easily be attached to these reactive alkyne groups via standard click chemistry. Moreover, DNA tags can also be designed with an orthogonal methyltetrazine (mTet) moiety for downstream coupling to a TCO-derivatized sequencing substrate via an inverse iEDDA reaction. In preferred embodiments, immobilization of polypeptide analytes on a solid support occurs via nucleic acid hybridization to capture DNA molecules spatially separated on a solid support as described in US 2022/0049246 A1, incorporated herein by reference in its entirety.

In some exemplary embodiments, a fractional inactivation of recording tags associated with abundant or selected protein analytes immobilized on a solid support occurs as follows. The recording tags associated with an abundant or selected protein species can be inactivated (i.e., made non-extendable in subsequent NGPS encoding cycles) by performing an NGPA assay with a population of cognate binding agent, wherein a fraction of the population is associated with a “productive” coding tag and the remaining fraction of the population is associated with a “non-productive” coding tag. During the encoding step of NGPA, the “productive” coding tag information is transferred to the proximal recording tag associated with the bound protein; likewise, the “non-productive” coding tag information is either rendered non-transferrable to the recording tag, or transfers “non-productive” sequence information to the proximal recording tag to the bound protein. Non-productive is defined in the context of the downstream encoding steps using NGPA or NGPS. In the simplest implementation, a coding tag is rendered “non-productive” by using a terminal non-cognate spacer sequence, wherein the non-productive spacer sequence is not recognized by downstream encoding cycles. Alternatively, the “productive” coding tag information can introduce a new spacer sequence compatible with downstream encoding events; “non-productive” encoding, in this context, can be accomplished by transferring information from a “non-productive” coding tag containing the same spacer sequence as existing, or by failure to transfer any sequence information (e.g., complete absence of coding tag sequence on binder).

Numerous specific details are set forth in the following description in order to provide a thorough understanding of the present disclosure. These details are provided for the purpose of example and the claimed subject matter may be practiced according to the claims without some or all of these specific details. It is to be understood that other embodiments can be used and structural changes can be made without departing from the scope of the claimed subject matter. It should be understood that the various features and functionality described in one or more of the individual embodiments are not limited in their applicability to the particular embodiment with which they are described. They instead can be applied, alone or in some combination, to one or more of the other embodiments of the disclosure, whether or not such embodiments are described, and whether or not such features are presented as being a part of a described embodiment. For the purpose of clarity, technical material that is known in the technical fields related to the claimed subject matter has not been described in detail so that the claimed subject matter is not unnecessarily obscured.

All publications, including patent documents, scientific articles and databases, referred to in this application are incorporated by reference in their entireties for all purposes to the same extent as if each individual publication were individually incorporated by reference. Citation of the publications or documents is not intended as an admission that any of them is pertinent prior art, nor does it constitute any admission as to the contents or date of these publications or documents.

All headings are for the convenience of the reader and should not be used to limit the meaning of the text that follows the heading, unless so specified.

### Definitions

Unless defined otherwise, all technical and scientific terms used herein have the same meaning as is commonly understood by one of ordinary skill in the art to which the present disclosure belongs. If a definition set forth in this section is contrary to or otherwise inconsistent with a definition set forth in the patents, applications, published applications and other publications that are herein incorporated by reference, the definition set forth in this section prevails over the definition that is incorporated herein by reference.

As used herein, the singular forms “a,” “an” and “the” include plural referents unless the context clearly dictates otherwise. Thus, for example, reference to “a peptide” includes one or more peptides, or mixtures of peptides. Also, and unless specifically stated or obvious from context, as used herein, the term “or” is understood to be inclusive and covers both “or” and “and”.

The term “about” as used herein refers to the usual error range for the respective value readily known to the skilled person in this technical field. Reference to “about” a value or parameter herein includes (and describes) embodiments that are directed to that value or parameter per se. For example, description referring to “about X” includes description of “X.

Use of ordinal terms such as “first”, “second”, “third”, etc., in the claims to modify a claim element does not by itself connote any priority, precedence, or order of one claim element over another or the temporal order in which acts of a method are performed, but are used merely as labels to distinguish one claim element having a certain name from another element having a same name (but for use of the ordinal term) to distinguish the claim elements. Similarly, use of a), b), etc., or i), ii), etc. does not by itself connote any priority, precedence, or order of steps in the claims. Similarly, the use of these terms in the specification does not by itself connote any required priority, precedence, or order.

As used herein, the term “sample” refers to anything which may contain an analyte for which an analyte assay is desired. As used herein, a “sample” can be a solution, a suspension, liquid, powder, a paste, aqueous, non-aqueous or any combination thereof. In some embodiments, the sample is a biological sample. A biological sample of the present disclosure encompasses a sample in the form of a solution, a suspension, a liquid, a powder, a paste, an aqueous sample, or a non-aqueous sample. As used herein, a “biological sample” includes any sample obtained from a living or viral (or prion) source or other source of polypeptides and biomolecules, and includes any cell type or tissue of a subject from which nucleic acid, protein and/or other polypeptide can be obtained. The biological sample can be a sample obtained directly from a biological source or a sample that is processed. For example, isolated nucleic acids that are amplified constitute a biological sample. Biological samples include, but are not limited to, body fluids, such as blood, plasma, serum, cerebrospinal fluid, synovial fluid, urine and sweat, tissue and organ samples from animals and plants and processed samples derived therefrom. In some embodiments, the sample can be derived from a tissue or a body fluid, for example, a connective, epithelium, muscle or nerve tissue; a tissue selected from the group consisting of brain, lung, liver, spleen, bone marrow, thymus, heart, lymph, blood, bone, cartilage, pancreas, kidney, gall bladder, stomach, intestine, testis, ovary, uterus, rectum, nervous system, gland, and internal blood vessels; or a body fluid selected from the group consisting of blood, urine, saliva, bone marrow, sperm, an ascitic fluid, and subfractions thereof, e.g., serum or plasma.

The terms “level” or “levels” are used to refer to the presence and/or amount of a target, e.g., a substance or an organism that is part of the etiology of a disease or disorder, and can be determined qualitatively or quantitatively. A “qualitative” change in the target level refers to the appearance or disappearance of a target that is not detectable or is present in samples obtained from normal controls. A “quantitative” change in the levels of one or more targets refers to a measurable increase or decrease in the target levels when compared to a healthy control.

As used herein, the term “polypeptide” encompasses peptides and proteins, and refers to a molecule comprising a chain of two or more amino acids joined by peptide bonds. In some embodiments, a polypeptide comprises 2 to 50 amino acids. In some embodiments, a polypeptide does not comprise a secondary, tertiary, or higher structure. In some embodiments, the polypeptide is a protein. In some embodiments, a protein comprises 30 or more amino acids. In some embodiments, in addition to a primary structure, a protein comprises a secondary, tertiary, or higher structure. The amino acids of the polypeptides are most typically L-amino acids, but may also be D-amino acids, modified amino acids, amino acid analogs, amino acid mimetics, or any combination thereof. Polypeptides may be naturally occurring, synthetically produced, or recombinantly expressed. Polypeptides may be synthetically produced, isolated, recombinantly expressed, or be produced by a combination of methodologies as described above. Polypeptides may also comprise additional groups modifying the amino acid chain, for example, functional groups added via post-translational modification. The polymer may be linear or branched, it may comprise modified amino acids, and it may be interrupted by non-amino acids. The term also encompasses an amino acid polymer that has been modified naturally or by intervention; for example, disulfide bond formation, glycosylation, lipidation, acetylation, phosphorylation, or any other manipulation or modification, such as conjugation with a labeling component.

As used herein, the term “amino acid” refers to an organic compound comprising an amine group, a carboxylic acid group, and a side-chain specific to each amino acid, which serve as a monomeric subunit of a peptide. An amino acid includes the 20 standard, naturally occurring or canonical amino acids as well as non-standard amino acids. The standard, naturally-occurring (or natural) amino acids include Alanine (A or Ala), Cysteine (C or Cys), Aspartic Acid (D or Asp), Glutamic Acid (E or Glu), Phenylalanine (F or Phe), Glycine (G or Gly), Histidine (H or His), Isoleucine (I or Ile), Lysine (K or Lys), Leucine (L or Leu), Methionine (M or Met), Asparagine (N or Asn), Proline (P or Pro), Glutamine (Q or Gln), Arginine (R or Arg), Serine (S or Ser), Threonine (T or Thr), Valine (V or Val), Tryptophan (W or Trp), and Tyrosine (Y or Tyr). An amino acid may be an L-amino acid or a D-amino acid. Non-standard amino acids may be modified amino acids, amino acid analogs, amino acid mimetics, non-standard proteinogenic amino acids, or non-proteinogenic amino acids that occur naturally or are chemically synthesized. Examples of non-standard amino acids include, but are not limited to, selenocysteine, pyrrolysine, and N-formylmethionine, β-amino acids, Homo-amino acids, Proline and Pyruvic acid derivatives, 3-substituted alanine derivatives, glycine derivatives, ring-substituted phenylalanine and tyrosine derivatives, linear core amino acids, N-methyl amino acids. The term “amino acid residue” refers to an amino acid incorporated into a polypeptide that forms peptide bond(s) with neighboring amino acid(s).

As used herein, the term “post-translational modification” refers to modifications that occur on a peptide after its translation, e.g., translation by ribosomes, is complete. A post-translational modification may be a covalent chemical modification or enzymatic modification. Examples of post-translation modifications include, but are not limited to, acylation, acetylation, alkylation (including methylation), biotinylation, butyrylation, carbamylation, carbonylation, deamidation, deiminiation, diphthamide formation, disulfide bridge formation, eliminylation, flavin attachment, formylation, gamma-carboxylation, glutamylation, glycylation, glycosylation, glypiation, heme C attachment, hydroxylation, hypusine formation, iodination, isoprenylation, lipidation, lipoylation, malonylation, methylation, myristolylation, oxidation, palmitoylation, pegylation, phosphopantetheinylation, phosphorylation, prenylation, propionylation, retinylidene Schiff base formation, S-glutathionylation, S-nitrosylation, S-sulfenylation, selenation, succinylation, sulfination, ubiquitination, and C-terminal amidation. A post-translational modification includes modifications of the amino terminus and/or the carboxyl terminus of a peptide. Modifications of the terminal amino group include, but are not limited to, des-amino, N-lower alkyl, N-di-lower alkyl, and N-acyl modifications. Modifications of the terminal carboxy group include, but are not limited to, amide, lower alkyl amide, dialkyl amide, and lower alkyl ester modifications (e.g., wherein lower alkyl is C1-C4 alkyl). A post-translational modification also includes modifications, such as but not limited to those described above, of amino acids falling between the amino and carboxy termini. The term post-translational modification can also include peptide modifications that include one or more detectable labels.

The term “detectable label” as used herein refers to a substance which can indicate the presence of another substance when associated with it. The detectable label can be a substance that is linked to or incorporated into the substance to be detected. In some embodiments, a detectable label is suitable for allowing for detection and also quantification, for example, a detectable label that emitting a detectable and measurable signal. Examples of detectable labels include a dye, a fluorophore, a chromophore, a fluorescent nanoparticle (e.g. quantum dot), a radiolabel, an enzyme (e.g. alkaline phosphatase, luciferase or horseradish peroxidase), or a chemiluminescent or bioluminescent molecule.

As used herein, the term “binding agent” or the term “binder” can refer to a nucleic acid molecule, a peptide, a polypeptide, a protein, carbohydrate, or a small molecule that binds to, associates, unites with, recognizes, or combines with a binding target, e.g., a polypeptide or a component or feature of a polypeptide. A binding agent or a binder may form a covalent association or non-covalent association with the polypeptide or component or feature of a polypeptide. A binding agent or a binder may also be a chimeric binding agent or binder, composed of two or more types of molecules, such as a nucleic acid molecule-peptide chimeric binding agent or binder or a carbohydrate-peptide chimeric binding agent or binder. A binding agent or a binder may be a naturally occurring, synthetically produced, or recombinantly expressed molecule. A binding agent or a binder may bind to a single monomer or subunit of a polypeptide (e.g., a single amino acid of a polypeptide) or bind to a plurality of linked subunits of a polypeptide (e.g., a di-peptide, tri-peptide, or higher order peptide of a longer peptide, polypeptide, or protein molecule). A binding agent or a binder may bind to a linear molecule or a molecule having a three-dimensional structure (also referred to as conformation). For example, an antibody binding agent or binder may bind to linear peptide, polypeptide, or protein, or bind to a conformational peptide, polypeptide, or protein. A binding agent or a binder may bind to an N-terminal peptide, a C-terminal peptide, or an intervening peptide of a peptide, polypeptide, or protein molecule. A binding agent or a binder may bind to an N-terminal amino acid, C-terminal amino acid, or an intervening amino acid of a peptide molecule. A binding agent or a binder may preferably bind to a chemically modified or labeled amino acid (e.g., an amino acid that has been labeled (modified) by a functionalizing reagent) over a non-modified or unlabeled amino acid. For example, a binding agent or a binder may preferably bind to an amino acid that has been labeled or modified over an amino acid that is unlabeled or unmodified. A binding agent or a binder may bind to a post-translational modification of a peptide molecule. A binding agent or a binder may exhibit selective binding to a component or feature of a polypeptide (e.g., a binding agent may selectively bind to one of the 20 possible natural amino acid residues and bind with very low affinity or not at all to the other 19 natural amino acid residues). A binding agent or a binder may exhibit less selective binding, where the binding agent or binder is capable of binding or configured to bind to a plurality of components or features of a polypeptide (e.g., a binding agent or a binder may bind with similar affinity to two or more different amino acid residues). A binding agent or a binder may comprise a coding tag or a handle (e.g., configured to bind to or react with a nucleic acid such as a recording tag), which may be joined to a binding moiety of the binding agent or binder by a linker.

The term “essentially identical binding moiety” as used herein generally refers to comparison of the binding moiety of one binding agent to the binding moiety of another binding agent. Essentially identical binding moieties refer to situation where both binding moieties comprise an identical protein or essentially identical protein, having no more than 10% of conservative amino acid substitutions, while retaining the ability to bind to the same cognate component of the polypeptide analyte (essentially identical binding moieties are configured to bind to the same component of the polypeptide analyte).

In some embodiments, variants of a proteinaceous binding agent or binding moiety displaying only non-substantial or negligible differences in structure can be generated by making conservative amino acid substitutions in the engineered binding agent or binding moiety. By doing this, further engineered binding agent or binding moiety variants that comprise a sequence having at least 90% (90%, 91%, 92%, 93%, 94%, 95%, 96%, 97%, 98%, and 99%) sequence identity with the initial engineered binding agent or binding moiety sequences can be generated, retaining at least one functional activity of the engineered binding agent or binding moiety, e.g., ability to bind to a specific (cognate) component of the polypeptide analyte. Examples of conservative amino acid changes are known in the art. Examples of non-conservative amino acid changes that are likely to cause major changes in protein structure are those that cause substitution of (a) a hydrophilic residue, e.g., serine or threonine, for (or by) a hydrophobic residue, e.g., leucine, isoleucine, phenylalanine, valine or alanine; (b) a cysteine or proline for (or by) any other residue; (c) a residue having an electropositive side chain, e.g., lysine, arginine, or histidine, for (or by) an electronegative residue, e.g., glutamic acid or aspartic acid; or (d) a residue having a bulky side chain, e.g., phenylalanine, for (or by) one not having a side chain, e g., glycine. Methods of making targeted amino acid substitutions, deletions, truncations, and insertions are generally known in the art. For example, amino acid sequence variants can be prepared by mutations in the DNA. Methods for polynucleotide alterations are well known in the art, for example, Kunkel et al. (1987) Methods in Enzymol. 154:367-382; U.S. Pat. No. 4,873,192 and the references cited therein.

The term “specifically binding” as used herein generally refers to an engineered binder (binding agent) that binds to a cognate target polypeptide analyte (such as polypeptide analyte) or a portion thereof more readily than it would bind to a random, non-cognate polypeptide analyte. The term “specificity” is used herein to qualify the relative affinity by which an engineered binder binds to a cognate target polypeptide analyte. Specific binding typically means that an engineered binder binds to a cognate target polypeptide analyte at least or about twice more likely that to a random, non-cognate polypeptide analyte (e.g., a 2:1 ratio of specific to non-specific binding). Non-specific binding refers to background binding, and is the amount of signal that is produced in a binding assay with an engineered binder when the cognate target polypeptide is not present in the assay. In some embodiments, specific binding will be at least three times the standard deviation of the background signal. In some specific embodiments, specific binding refers to binding between an engineered binder and an N-terminally modified target polypeptide with a dissociation constant (Kd) of 200 nM or less.

Binding agents that are specific for or bind specifically to a target polypeptide analyte avoid binding to a significant percentage of non-target substances, e.g., non-target substances present in a testing sample. In some embodiments, binding agents of the present disclosure avoid binding greater than about 90% of non-target substances, although higher percentages are clearly contemplated and preferred. For example, binding agents of the present disclosure avoid binding about 91%, about 92%, about 93%, about 94%, about 95%, about 96%, about 97%, about 98%, about 99% or more of non-target substances. In other embodiments, binding agents of the present disclosure avoid binding greater than about 10%, 20%, 30%, 40%, 50%, 60%, or 70%, or greater than about 75%, or greater than about 80%, or greater than about 85% of non-target substances.

As used herein, the term “linker” refers to one or more of a nucleotide, a nucleotide analog, an amino acid, a peptide, a polypeptide, a polymer, or a non-nucleotide chemical moiety that is used to join two molecules. A linker may be used to join a binding agent with a coding tag, a recording tag with a polypeptide, a polypeptide with a support, a recording tag with a solid support, etc. In certain embodiments, a linker joins two molecules via enzymatic reaction or chemistry reaction (e.g., a click chemistry reaction). In certain embodiments, the nucleic acid recording tag is associated directly or indirectly to the polypeptide analyte via a non-nucleotide chemical moiety.

The term “ligand” as used herein refers to any molecule or moiety connected to the compounds described herein. “Ligand” may refer to one or more ligands attached to a compound. In some embodiments, the ligand is a pendant group or binding site (e.g., the site to which the binding agent binds).

The terminal amino acid at one end of a peptide or polypeptide chain that has a free amino group is referred to herein as the “N-terminal amino acid” (NTAA). The terminal amino acid at the other end of the chain that has a free carboxyl group is referred to herein as the “C-terminal amino acid” (CTAA). The amino acids making up a peptide may be numbered in order, with the peptide being “n” amino acids in length. As used herein, NTAA is considered the nth amino acid (also referred to herein as the “n NTAA”). Using this nomenclature, the next amino acid is the n−1 amino acid, then the n−2 amino acid, and so on down the length of the peptide from the N-terminal end to C-terminal end. In certain embodiments, an NTAA, CTAA, or both may be modified or labeled with a moiety or a chemical moiety.

As used herein, the term “barcode” refers to a nucleic acid molecule of about 2 to about 30 bases (e.g., 2, 3, 4, 5, 6, 7, 8, 9, 10, 11, 12, 13, 14, 15, 16, 17, 18, 19, 20, 21, 22, 23, 24, 25, 26, 27, 28, 29 or 30 bases) providing a unique identifier tag or origin information for a polypeptide, a binding agent, a set of binding agents from a binding cycle, a sample polypeptides, a set of samples, polypeptides within a compartment (e.g., droplet, bead, or separated location), polypeptides within a set of compartments, a fraction of polypeptides, a set of polypeptide fractions, a spatial region or set of spatial regions, a library of polypeptides, or a library of binding agents. A barcode can be an artificial sequence or a naturally occurring sequence. In certain embodiments, each barcode within a population of barcodes is different. In other embodiments, a portion of barcodes in a population of barcodes is different, e.g., at least about 10%, 15%, 20%, 25%, 30%, 35%, 40%, 45%, 50%, 55%, 60%, 65%, 70%, 75%, 80%, 85%, 90%, 95%, 97%, or 99% of the barcodes in a population of barcodes is different. A population of barcodes may be randomly generated or non-randomly generated. In certain embodiments, a population of barcodes are error-correcting or error-tolerant barcodes. Barcodes can be used to computationally deconvolute the multiplexed sequencing data and identify sequence reads derived from an individual polypeptide, sample, library, etc. A barcode can also be used for deconvolution of a collection of polypeptides that have been distributed into small compartments for enhanced mapping. For example, rather than mapping a peptide back to the proteome, the peptide is mapped back to its originating protein molecule or protein complex.

As used herein, the term “coding tag” refers to a polynucleotide with any suitable length, e.g., a nucleic acid molecule of about 2 bases to about 100 bases, including any integer including 2 and 100 and in between, that comprises identifying information for its associated binding agent. A “coding tag” may also be made from a “sequenceable polymer” (see, e.g., Niu et al., 2013, Nat. Chem. 5:282-292; Roy et al., 2015, Nat. Commun. 6:7237; Lutz, 2015, Polypeptides 48:4759-4767; each of which are incorporated by reference in its entirety). A coding tag may comprise an encoder sequence, which is optionally flanked by one spacer on one side or optionally flanked by a spacer on each side. A coding tag may also be comprised of an optional UMI and/or an optional binding cycle-specific barcode. A coding tag may be single stranded or double stranded. A double stranded coding tag may comprise blunt ends, overhanging ends, or both. A coding tag may refer to the coding tag that is directly attached to a binding agent, to a complementary sequence hybridized to the coding tag directly attached to a binding agent (e.g., for double stranded coding tags), or to coding tag information present in an extended recording tag. In certain embodiments, a coding tag may further comprise a binding cycle specific spacer or barcode, a unique molecular identifier, a universal priming site, or any combination thereof.

As used herein, the term “spacer” (Sp) refers to a nucleic acid molecule of about 1 base to about 20 bases (e.g., 1, 2, 3, 4, 5, 6, 7, 8, 9, 10, 11, 12, 13, 14, 15, 16, 17, 18, 19, or 20 bases) in length that is present on a terminus of a recording tag or coding tag. In certain embodiments, a spacer sequence flanks an encoder sequence of a coding tag on one end or both ends. Following binding of a binding agent to a polypeptide, annealing between complementary spacer sequences on their associated coding tag and recording tag, respectively, allows transfer of binding information through a primer extension reaction or ligation to the recording tag, coding tag, or a di-tag construct. Sp′ refers to spacer sequence complementary to Sp. Preferably, spacer sequences within a library of binding agents possess the same number of bases. A common (shared or identical) spacer may be used in a library of binding agents. A spacer sequence may have a “cycle specific” sequence in order to track binding agents used in a particular binding cycle. The spacer sequence (Sp) can be constant across all binding cycles, be specific for a particular class of polypeptides, or be binding cycle number specific. Polypeptide class-specific spacers permit annealing of a cognate binding agent's coding tag information present in an extended recording tag from a completed binding/extension cycle to the coding tag of another binding agent recognizing the same class of polypeptides in a subsequent binding cycle via the class-specific spacers. Only the sequential binding of correct cognate pairs results in interacting spacer elements and effective primer extension. A spacer sequence may comprise sufficient number of bases to anneal to a complementary spacer sequence in a recording tag to initiate a primer extension (also referred to as polymerase extension) reaction, or provide a “splint” for a ligation reaction, or mediate a “sticky end” ligation reaction. In some embodiments, a spacer sequence disclosed herein comprises a nucleic acid sequence, and an extended recording tag or extended coding tag comprising one or more spacer sequences (or complements thereof) is sequenceable.

As used herein, the term “recording tag” refers to a moiety, e.g., a chemical coupling moiety, a nucleic acid molecule, or a sequenceable polymer molecule (see, e.g., Niu et al., 2013, Nat. Chem. 5:282-292; Roy et al., 2015, Nat. Commun. 6:7237; Lutz, 2015, Polypeptides 48:4759-4767; each of which are incorporated by reference in its entirety) to which identifying information of a coding tag can be transferred, or from which identifying information about the polypeptide associated with the recording tag can be transferred to the coding tag. Identifying information can comprise any information characterizing a molecule such as information pertaining to sample, fraction, partition, spatial location, interacting neighboring molecule(s), cycle number, etc. Additionally, the presence of UMI can also be classified as identifying information. In certain embodiments, after a binding agent binds to a polypeptide, information from a coding tag linked to a binding agent can be transferred to the recording tag associated with the polypeptide while the binding agent is bound to the polypeptide. In other embodiments, after a binding agent binds to a polypeptide, information from a recording tag associated with the polypeptide can be transferred to the coding tag linked to the binding agent while the binding agent is bound to the polypeptide. A recording tag may be directly linked to a polypeptide, linked to a polypeptide via a multifunctional linker, or associated with a polypeptide by virtue of its proximity (or co-localization) on a support. A recording tag may be linked via its 5′ end or 3′ end or at an internal site, as long as the linkage is compatible with the method used to transfer coding tag information to the recording tag or vice versa. A recording tag may further comprise other functional components, e.g., a universal priming site, unique molecular identifier, a barcode (e.g., a sample barcode, a fraction barcode, spatial barcode, a compartment tag, etc.), a spacer sequence that is complementary to a spacer sequence of a coding tag, or any combination thereof. The spacer sequence of a recording tag is preferably at the 3′-end of the recording tag in embodiments where polymerase extension is used to transfer coding tag information to the recording tag.

As used herein, the term “unique molecular identifier” or “UMI” refers to a nucleic acid molecule of about 3 to about 40 bases (3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, or 40 bases) in length providing a unique identifier tag for each polypeptide, polypeptide or binding agent to which the UMI is linked. A polypeptide UMI can be used to computationally deconvolute sequencing data from a plurality of extended recording tags to identify extended recording tags that originated from an individual polypeptide. A polypeptide UMI can be used to accurately count originating polypeptide molecules by collapsing NGS reads to unique UMIs. A binding agent UMI can be used to identify each individual molecular binding agent that binds to a particular polypeptide. For example, a UMI can be used to identify the number of individual binding events for a binding agent specific for a single amino acid that occurs for a particular peptide molecule. It is understood that when UMI and barcode are both referenced in the context of a binding agent or polypeptide, that the barcode refers to identifying information other that the UMI for the individual binding agent or polypeptide (e.g., sample barcode, compartment barcode, binding cycle barcode).

As used herein, the term “universal priming site” or “universal primer” or “universal priming sequence” refers to a nucleic acid molecule, which may be used for library amplification and/or for sequencing reactions. A universal priming site may include, but is not limited to, a priming site (primer sequence) for PCR amplification, flow cell adaptor sequences that anneal to complementary oligonucleotides on flow cell surfaces enabling bridge amplification in some next generation sequencing platforms, a sequencing priming site, or a combination thereof. Universal priming sites can be used for other types of amplification, including those commonly used in conjunction with next generation digital sequencing. For example, extended recording tag molecules may be circularized and a universal priming site used for rolling circle amplification to form DNA nanoballs that can be used as sequencing templates (Drmanac et al., 2009, Science 327:78-81).

As used herein, the term “extended recording tag” refers to a recording tag to which information of at least one binding agent's coding tag (or its complementary sequence) has been transferred following binding of the binding agent to a polypeptide. Information of the coding tag may be transferred to the recording tag directly (e.g., ligation) or indirectly (e.g., primer extension). Information of a coding tag may be transferred to the recording tag enzymatically or chemically. An extended recording tag may comprise binding agent information of 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 35, 40, 45, 50, 55, 60, 65, 70, 75, 80, 85, 90, 95, 100, 200 or more coding tags. The base sequence of an extended recording tag may reflect the temporal and sequential order of binding of the binding agents identified by their coding tags, may reflect a partial sequential order of binding of the binding agents identified by the coding tags, or may not reflect any order of binding of the binding agents identified by the coding tags. In certain embodiments, the coding tag information present in the extended recording tag represents with at least 25%, 30%, 35%, 40%, 45%, 50%, 55%, 60%, 65%, 70%, 75%, 80%, 85%, 90%, 91%, 92%, 93%, 94%, 95%, 96%, 97%, 98%, 99%, or 100% identity the polypeptide sequence being analyzed. In certain embodiments where the extended recording tag does not represent the polypeptide sequence being analyzed with 100% identity, errors may be due to off-target binding by a binding agent, or to a “missed” binding cycle (e.g., because a binding agent fails to bind to a polypeptide during a binding cycle, because of a failed primer extension reaction), or both.

As used herein, the term “support” or “solid support” can include any material suitable for an assay disclosed herein (e.g., an NGPA or NGPA assay), such as a suitable solid material, including porous and non-porous materials, to which a polypeptide can be associated directly or indirectly, by any means known in the art, including covalent and non-covalent interactions, or any combination thereof. A solid support may be two-dimensional (e.g., planar surface) or three-dimensional (e.g., gel matrix or bead). A solid support can be any support surface including, but not limited to, a bead, a microbead, an array, a glass surface, a silicon surface, a plastic surface, a filter, a membrane, a PTFE membrane, a silicon wafer chip, a flow through chip, a flow cell, a biochip including signal transducing electronics, a channel, a microtiter well, an ELISA plate, a spinning interferometry disc, a nitrocellulose-based polymer surface, a polymer matrix, a nanoparticle, or a microsphere. Materials for a support, such as a bead, include but are not limited to acrylamide, agarose, cellulose, dextran, nitrocellulose, glass, gold, quartz, polystyrene, polyethylene vinyl acetate, polypropylene, polyester, polymethacrylate, polyacrylate, polyethylene, polyethylene oxide, polysilicates, polycarbonates, poly vinyl alcohol (PVA), Teflon, fluorocarbons, nylon, silicon rubber, polyanhydrides, polyglycolic acid, polyvinylchloride, polylactic acid, polyorthoesters, functionalized silane, polypropylfumerate, collagen, glycosaminoglycans, polyamino acids, dextran, or any combination thereof. In some embodiments, a bead can be a porous bead, such as a cross-linked agarose bead, which provides a high surface area for interaction with molecules such as proteins. In some embodiments, a bead can be a gel bead. Exemplary supports further include thin film, membrane, bottles, dishes, fibers, woven fibers, shaped polymers such as tubes, particles, beads, microspheres, microparticles, or any combination thereof. For example, when solid support is a bead, the bead can include, but is not limited to, a ceramic bead, a polystyrene bead, a polymer bead, a polyacrylate bead, a methylstyrene bead, an agarose bead, a cellulose bead, a dextran bead, an acrylamide bead (e.g., a polyacrylamide gel bead), a solid core bead, a porous bead, a paramagnetic bead, a glass bead, a controlled pore bead, a silica-based bead, or any combinations thereof. A bead may be spherical or an irregularly shaped. A bead or support may be porous. A bead's size may range from nanometers, e.g., 100 nm, to millimeters, e.g., 1 mm. In certain embodiments, beads range in size from about 0.2 micron to about 200 microns, or from about 0.5 micron to about 5 micron. In some embodiments, beads can be about 1, 1.5, 2, 2.5, 2.8, 3, 3.5, 4, 4.5, 5, 5.5, 6, 6.5, 7, 7.5, 8, 8.5, 9, 9.5, 10, 10.5, 15, or 20 μm in diameter. In certain embodiments, “a bead” solid support may refer to an individual bead or a plurality of beads. In some embodiments, the solid support is a nanoparticle. In certain embodiments, the nanoparticles range in size from about 1 nm to about 500 nm in diameter, for example, between about 1 nm and about 20 nm, between about 1 nm and about 50 nm, between about 1 nm and about 100 nm, between about 10 nm and about 50 nm, between about 10 nm and about 100 nm, between about 10 nm and about 200 nm, between about 50 nm and about 100 nm, between about 50 nm and about 150, between about 50 nm and about 200 nm, or between about 200 nm and about 500 nm in diameter. In some embodiments, the nanoparticles can be about 10 nm, about 50 nm, about 100 nm, about 150 nm, about 200 nm, about 300 nm, or about 500 nm in diameter. In some embodiments, the nanoparticles are less than about 200 nm in diameter.

In some embodiments, a support is a porous bead. Porous beads are beads having pore structures or empty channels through which molecules can pass. In some embodiments, porous beads are microspheres. In some embodiments, porous beads are beads having pore structures or empty channels through which polypeptides and binding agents can pass. Using such porous beads is advantageous, since porous beads have significantly increased surface area, where antibodies may be attached and contacted with binding agents. Multiple examples of porous beads are known in the art, such as made of crosslinked polysaccharide polymers (e.g., sepharose), polymer microspheres and silica beads. Exemplary polymer non-agarose-based microspheres comprise TSKgel Ether-5 PW beads (made from polymethacrylate material bonded with polyether groups and having ˜100 nm pore size) and POROS beads (Thermo Scientific; incompressible beads with cross-linked polystyrene-divinylbenzene backbone having ˜100-360 nm pore size). Exemplary size ranges for porous beads comprise 20-50 um, 20-70 um, and 70-50 um. Exemplary pore size ranges comprise 20-70 nm. 30-70 nm, 40-70 nm, 70-100 nm and 100-360 nm.

As used herein, the term “nucleic acid molecule” or “polynucleotide” refers to a single- or double-stranded polynucleotide containing deoxyribonucleotides or ribonucleotides that are linked by 3′-5′ phosphodiester bonds, as well as polynucleotide analogs. A nucleic acid molecule includes, but is not limited to, DNA, RNA, and cDNA. A polynucleotide analog may possess a backbone other than a standard phosphodiester linkage found in natural polynucleotides and, optionally, a modified sugar moiety or moieties other than ribose or deoxyribose. Polynucleotide analogs contain bases capable of hydrogen bonding by Watson-Crick base pairing to standard polynucleotide bases, where the analog backbone presents the bases in a manner to permit such hydrogen bonding in a sequence-specific fashion between the oligonucleotide analog molecule and bases in a standard polynucleotide. Examples of polynucleotide analogs include, but are not limited to xeno nucleic acid (XNA), bridged nucleic acid (BNA), glycol nucleic acid (GNA), peptide nucleic acids (PNAs), morpholino polynucleotides, locked nucleic acids (LNAs), threose nucleic acid (TNA), 2′-O-Methyl polynucleotides, 2′-O-alkyl ribosyl substituted polynucleotides, phosphorothioate polynucleotides, and boronophosphate polynucleotides. A polynucleotide analog may possess purine or pyrimidine analogs, including for example, 7-deaza purine analogs, 8-halopurine analogs, 5-halopyrimidine analogs, or universal base analogs that can pair with any base, including hypoxanthine, nitroazoles, isocarbostyril analogues, azole carboxamides, and aromatic triazole analogues, or base analogs with additional functionality, such as a biotin moiety for affinity binding. In some embodiments, the nucleic acid molecule or oligonucleotide is a modified oligonucleotide. In some embodiments, the nucleic acid molecule or oligonucleotide is a DNA with pseudo-complementary bases, a DNA with protected bases, an RNA molecule, a BNA molecule, an XNA molecule, a LNA molecule, a PNA molecule, or a morpholino DNA, or a combination thereof. In some embodiments, the nucleic acid molecule or oligonucleotide is backbone modified, sugar modified, or nucleobase modified. In some embodiments, the nucleic acid molecule or oligonucleotide has nucleobase protecting groups such as Alloc, electrophilic protecting groups such as thiranes, acetyl protecting groups, nitrobenzyl protecting groups, sulfonate protecting groups, or traditional base-labile protecting groups.

As used herein, “nucleic acid sequencing” means the determination of the order of nucleotides in a nucleic acid molecule or a sample of nucleic acid molecules. Similarly, “polypeptide sequencing” means the determination of the identity and order of at least a portion of amino acids in the polypeptide molecule or in a sample of polypeptide molecules.

As used herein, “next generation sequencing” refers to high-throughput sequencing methods that allow the sequencing of millions to billions of molecules in parallel. Examples of next generation sequencing methods include sequencing by synthesis, sequencing by ligation, sequencing by hybridization, polony sequencing, ion semiconductor sequencing, and pyrosequencing. By attaching primers to a solid support and a complementary sequence to a nucleic acid molecule, a nucleic acid molecule can be hybridized to the solid support via the primer and then multiple copies can be generated in a discrete area on the solid support by using polymerase to amplify (these groupings are sometimes referred to as polymerase colonies or polonies). Consequently, during the sequencing process, a nucleotide at a particular position can be sequenced multiple times (e.g., hundreds or thousands of times)—this depth of coverage is referred to as “deep sequencing.” Examples of high throughput nucleic acid sequencing technology include platforms provided by Illumina, BGI, Qiagen, Thermo-Fisher, and Roche, including formats such as parallel bead arrays, sequencing by synthesis, sequencing by ligation, capillary electrophoresis, electronic microchips, “biochips.” microarrays, parallel microchips, and single-molecule arrays (See e.g., Service, Science (2006) 311:1544-1546).

As used herein, “analyzing” the polypeptide means to identify, detect, quantify, characterize, distinguish, or a combination thereof, all or a portion of the components of the polypeptide. For example, analyzing a peptide, polypeptide, or protein includes determining all or a portion of the amino acid sequence (contiguous or non-continuous) of the peptide. Analyzing a polypeptide also includes partial identification of a component of the polypeptide. For example, partial identification of amino acids in the polypeptide protein sequence can identify an amino acid in the protein as belonging to a subset of possible amino acids. Analysis typically begins with analysis of the n NTAA, and then proceeds to the next amino acid of the peptide (i.e., n−1, n−2, n−3, and so forth). This is accomplished by elimination of the n NTAA, thereby converting the n−1 amino acid of the peptide to an N-terminal amino acid (referred to herein as the “n−1 NTAA”). Analyzing the peptide may also include determining the presence and frequency of post-translational modifications on the peptide, which may or may not include information regarding the sequential order of the post-translational modifications on the peptide. Analyzing the peptide may also include determining the presence and frequency of epitopes in the peptide, which may or may not include information regarding the sequential order or location of the epitopes within the peptide. Analyzing the peptide may include combining different types of analysis, for example obtaining epitope information, amino acid sequence information, post-translational modification information, or any combination thereof.

The term “sequence identity” is a measure of identity between polypeptides at the amino acid level, and a measure of identity between nucleic acids at nucleotide level. The polypeptide sequence identity may be determined by comparing the amino acid sequence in a given position in each sequence when the sequences are aligned. Similarly, the nucleic acid sequence identity may be determined by comparing the nucleotide sequence in a given position in each sequence when the sequences are aligned. “Sequence identity” means the percentage of identical subunits at corresponding positions in two sequences when the two sequences are aligned to maximize subunit matching, i.e., taking into account gaps and insertions. For example, the BLAST algorithm (NCBI) calculates percent sequence identity and performs a statistical analysis of the similarity and identity between the two sequences. The software for performing BLAST analysis is publicly available through the National Center for Biotechnology Information (NCBI) website.

The term “unmodified” (also “wild-type” or “native”) as used herein is used in connection with biological materials such as nucleic acid molecules and proteins (e.g., cleavase), refers to those which are found in nature and not modified by human intervention.

The term “modified” or “engineered” (or “variant”, or “mutant”) as used in reference to nucleic acid molecules and protein molecules, e.g., an engineered DNA polymerase, implies that such molecules are created by human intervention and/or they are non-naturally occurring. The variant, mutant or engineered DNA polymerase is a polypeptide having an altered amino acid sequence, relative to an unmodified or wild-type protein, such as starting DNA polymerase, or a portion thereof. An engineered enzyme is a polypeptide which differs from a wild-type enzyme scaffold sequence, or a portion thereof, by one or more amino acid substitutions, deletions, additions, or combinations thereof. An engineered DNA polymerase generally exhibits at least 70%, 80%, 85%, 86%, 87%, 88%, 89%, 90%, 91%, 92%, 93%, 94%, 95%, 96%, 97%, 98%, 99% or more sequence identity to a corresponding wild-type starting DNA polymerase scaffold. Non-naturally occurring amino acids as well as naturally occurring amino acids are included within the scope of permissible substitutions or additions. A variant or engineered DNA polymerase denotes a composition and not necessarily a product produced by any given process. A variety of techniques including genetic selection, protein engineering, recombinant methods, chemical synthesis, or combinations thereof, may be employed.

The terms “corresponding to position(s)” or “position(s) . . . with reference to position(s)” of or within a polypeptide or a polynucleotide, such as recitation that nucleotides or amino acid positions “correspond to” nucleotides or amino acid positions of a disclosed sequence, such sequence set forth in the Sequence Listing, refers to nucleotides or amino acid positions identified in the polynucleotide or in the polypeptide upon alignment with the disclosed sequence using a standard alignment algorithm, such as the BLAST algorithm (NCBI). By aligning the sequences, one skilled in the art can identify corresponding residues in a given polypeptide, for example, by using conserved and identical amino acid residues in the alignment as guides. Similarly, one skilled in the art can identify any given amino acid residue in a given polypeptide at a position corresponding to a particular position of a reference sequence, such as set forth in the Sequence Listing, by performing alignment of the polypeptide sequence with the reference sequence (for example, by BLASTP publicly available through the NCBI website), matching the corresponding position of the reference sequence with the position in polypeptide sequence and thus identifying the amino acid residue within the polypeptide.

The term “template” as used herein refers to a double-stranded or single-stranded nucleic acid molecule which is to be amplified, synthesized or sequenced. In the case of a double-stranded DNA molecule, denaturation of its strands to form a first and a second strand is performed before these molecules may be amplified, synthesized or sequenced. A primer, complementary to a portion of a template is hybridized under appropriate conditions and the polymerase of the disclosure may then synthesize a molecule complementary to said template or a portion thereof. Mismatch incorporation or strand slippage during the synthesis or extension of the newly synthesized molecule may result in one or a number of mismatched nucleotide pairs.

As used herein “amplification” refers to any in vitro method for increasing the number of copies of a nucleotide sequence with the use of a DNA polymerase. Nucleic acid amplification results in the incorporation of nucleotides into a DNA molecule or primer thereby forming a new DNA molecule complementary to a DNA template. The formed DNA molecule and its template can be used as templates to synthesize additional DNA molecules.

The terms “hybridization” and “hybridizing” refers to the pairing of two complementary single-stranded nucleic acid molecules (RNA and/or DNA) to give a double-stranded molecule. As used herein, two nucleic acid molecules may be hybridized, although the base pairing is not completely complementary. Accordingly, mismatched bases do not prevent hybridization of two nucleic acid molecules provided that appropriate conditions, well known in the art, are used. In the present disclosure, the term “hybridization” refers particularly to hybridization of an oligonucleotide to a template molecule.

As used herein, the term “primer extension”, also referred to as “polymerase extension”, refers to a reaction catalyzed by a nucleic acid polymerase (e.g., DNA polymerase) whereby a nucleic acid molecule (e.g., oligonucleotide primer, spacer sequence) that anneals to a complementary strand is extended by the polymerase, using the complementary strand as template.

The term “3′→5′ exonuclease activity” refers to an enzymatic activity associated with DNA polymerases and is involved in a DNA replication “editing” or correction mechanism during template extension.

A “DNA polymerase substantially reduced in 3′-to-5′ (or 3′-5′) exonuclease activity” is defined herein as a DNA polymerase having a 3′-5′ exonuclease specific activity which is less than about 1 unit/mg protein, or preferably about or less than 0.1 units/mg protein. Non-limiting examples of commercially available DNA polymerases that have substantially reduced 3′->5′ exonuclease activity include Taq. Klenow fragment DNA polymerase, Tne(exo-), Tma(exo-), Pfu (exo-) DNA polymerases, and mutants, variants and derivatives thereof. Alternatively, a DNA polymerase with substantially reduced 3′-to-5′ exonuclease activity can be obtained by introducing mutation(s) to a DNA polymerase having 3′->5′ exonuclease activity, and can be defined as a mutated DNA polymerase that has about or less than 10%, or preferably about or less than 1%, of the 3′-5′ exonuclease activity of the corresponding unmutated, wildtype enzyme.

It is understood that aspects and embodiments of the disclosure described herein include “consisting of” and/or “consisting essentially of” aspects and embodiments.

Throughout this disclosure, various aspects of this disclosure are presented in a range format. It should be understood that the description in range format is merely for convenience and brevity and should not be construed as an inflexible limitation on the scope of the disclosure. Accordingly, the description of a range should be considered to have specifically disclosed all the possible sub-ranges as well as individual numerical values within that range. For example, description of a range such as from 1 to 6 should be considered to have specifically disclosed sub-ranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

Several elements and steps for performing the disclosed methods are further explained below.

### Attachment to the Support

In some embodiments, the target (e.g., polypeptide) is joined to a support before performing the binding reaction. In some cases, it is desirable to use a support with a large carrying capacity to immobilize a large number of targets (e.g., polypeptides). In some embodiments, it is preferred to immobilize the targets using a three-dimensional support (e.g., a porous matrix or a porous bead). For example, the preparation of the targets including joining the target to a support is performed prior to performing the binding reaction. In some examples, the preparation of the target including joining the polypeptide to nucleic acid molecule or a oligonucleotide may be performed prior to or after immobilizing the target. In some embodiments, a plurality of targets are attached to a support prior to the binding reaction and contacting with a binding agent.

In some embodiments, the support may comprise any suitable solid material, including porous and non-porous materials, to which a polypeptide, e.g., a polypeptide, can be associated directly or indirectly, by any means known in the art, including covalent and non-covalent interactions, or any combination thereof.

Various reactions may be used to attach the polypeptide analytes to a support (e.g., a solid or a porous support). The polypeptides may be attached directly or indirectly to the support. In some cases, the polypeptides are attached to the support via a nucleic acid. Exemplary reactions include click chemistry reactions, such as the copper catalyzed reaction of an azide and alkyne to form a triazole (Huisgen 1, 3-dipolar cycloaddition), strain-promoted azide alkyne cycloaddition (SPAAC), reaction of a diene and dienophile (Diels-Alder), strain-promoted alkyne-nitrone cycloaddition, reaction of a strained alkene with an azide, tetrazine or tetrazole, alkene and azide [3+2] cycloaddition, alkene and tetrazine inverse electron demand Diels-Alder (IEDDA) reaction (e.g., m-tetrazine (mTet) or phenyl tetrazine (pTet) and trans-cyclooctene (TCO); or pTet and an alkene), alkene and tetrazole photoreaction, Staudinger ligation of azides and phosphines, and various displacement reactions, such as displacement of a leaving group by nucleophilic attack on an electrophilic atom (Horisawa 2014, Knall, Hollauf et al. 2014). Exemplary displacement reactions include reaction of an amine with: an activated ester; an N-hydroxysuccinimide ester; an isocyanate; an isothioscyanate, an aldehyde, an epoxide, or the like. In some embodiments, iEDDA click chemistry is used for immobilizing polypeptides to a support since it is rapid and delivers high yields at low input concentrations. In another embodiment, m-tetrazine rather than tetrazine is used in an iEDDA click chemistry reaction, as m-tetrazine has improved bond stability. In another embodiment, phenyl tetrazine (pTet) is used in an iEDDA click chemistry reaction. In one case, a polypeptide is labeled with a bifunctional click chemistry reagent, such as alkyne-NHS ester (acetylene-PEG-NHS ester) reagent or alkyne-benzophenone to generate an alkyne-labeled polypeptide. In some embodiments, an alkyne can also be a strained alkyne, such as cyclooctynes including Dibenzocyclooctyl (DBCO).

Similar methods (e.g., click chemistry reactions, bioorthogonal reactions) can be used to attach the polypeptide analyte to the associated nucleic acid recording tag, or to attach the binding agent to the associated nucleic acid coding tag. Such attachments can be achieved by introducing reactive moiety or moieties on one or on both attachment partners.

In some embodiments of the disclosed methods, a plurality of different polypeptides is immobilized on a solid support, wherein each polypeptide of the plurality of different polypeptides is associated with a nucleic acid recording tag. Various possible ways exist for association between an immobilized polypeptide and the associated nucleic acid recording tag. A recording tag may be directly linked to the polypeptide, linked to a polypeptide via a linker, via a multifunctional linker, or associated with a polypeptide by virtue of its proximity (or co-localization) on the support (see also section “Recording Tag and immobilization methods” below). In some embodiments, the recording tag is attached to the support, and the polypeptide is immobilized on the support via the recording tag. In some embodiments, a linker is attached to the support, and the polypeptide and the recording tag are independently attached to the linker, thereby generating immobilization on the support and association of the polypeptide with the recording tag. Other immobilization and association variants are possible.

In certain embodiments where multiple targets are immobilized on the same support, the target molecules can be spaced appropriately to accommodate methods of performing the binding reaction and any downstream analysis steps to be used to assess the target. For example, it may be advantageous to space the target molecules that optimally to allow a nucleic acid-based method for assessing and sequencing the proteins to be performed. In some embodiments, the method for assessing and sequencing protein targets involve a binding agent which binds to the target molecules and the binding agent comprises a coding tag with information that is transferred to a nucleic acid attached to the target molecules. In some cases, spacing of the targets on the support is determined based on the consideration that information transfer from a coding tag of a binding agent bound to one target molecule may reach a neighboring molecule.

In some embodiments, the surface of the support is passivated (blocked). A “passivated” surface refers to a surface that has been treated with outer layer of material. Methods of passivating surfaces include standard methods from the fluorescent single molecule analysis literature, including passivating surfaces with polymer like polyethylene glycol (PEG) (Pan et al., 2015, Phys. Biol. 12:045006), polysiloxane (e.g., Pluronic F-127), star polymers (e.g., star PEG) (Groll et al., 2010, Methods Enzymol. 472:1-18), hydrophobic dichlorodimethylsilane (DDS)+self-assembled Tween-20 (Hua et al., 2014, Nat. Methods 11:1233-1236), diamond-like carbon (DLC), DLC+PEG (Stavis et al., 2011, Proc. Natl. Acad. Sci. USA 108:983-988), and zwitterionic moiety (e.g., U.S. Patent Application Publication US 2006/0183863). In addition to covalent surface modifications, a number of passivating agents can be employed as well including surfactants like Tween-20, polysiloxane in solution (Pluronic series), poly vinyl alcohol (PVA), and proteins like BSA and casein. Alternatively, density of polypeptides (e.g., proteins, polypeptide, or peptides) can be titrated on the surface or within the volume of a solid support by spiking a competitor or “dummy” reactive molecule when immobilizing the proteins, polypeptides or peptides to the solid support.

To control spacing of the immobilized targets on the support, the density of functional coupling groups for attaching the target (e.g., TCO or carboxyl groups (COOH)) may be titrated on the support surface. In some embodiments, multiple target molecules (e.g., polypeptides) are spaced apart on the surface or within the volume (e.g., porous supports) of a support such that adjacent molecules are spaced apart at a distance of about 50 nm to about 500 nm, or about 50 nm to about 200 nm, or about 50 nm to about 100 nm. In some embodiments, multiple molecules are spaced apart on the surface of a support with an average distance of at least 50 nm, at least 60 nm, at least 70 nm, at least 80 nm, at least 90 nm, at least 100 nm, at least 200 nm, or at least 500 nm. In some embodiments, multiple molecules are spaced apart on the surface of a support with an average distance of at least 50 nm. In some embodiments, molecules are spaced apart on the surface or within the volume of a support such that, empirically, the relative frequency of inter- to intra-molecular events (e.g., transfer of information) is <1:10; <1:100; <1:1,000; or <1:10,000. In some embodiments, the plurality of target molecules (e.g., polypeptides) is coupled on the support spaced apart at an average distance between two adjacent molecules which ranges from about 50 to 100 nm, from about 50 to 500 nm, from about 50 to 1,000 nm, from about 50 to 2,000 nm, from about 500 to 600 nm, from about 500 to 1,000 nm, from about 500 to 2,000 nm, from about 500 to 5,000 nm, from about 1,000 to 5,000 nm, or from about 3,000 to 5,000 nm.

In some embodiments, appropriate spacing of the target molecules (e.g., polypeptides) on the support is accomplished by titrating the ratio of available attachment molecules on the support surface. In some examples, the support surface (e.g., bead surface) is functionalized with a carboxyl group (COOH) which is treated with an activating agent (e.g., activating agent is EDC and Sulfo-NHS). In some examples, the support surface (e.g., bead surface) comprises NHS moieties. In some embodiments, a mixture of mPEGn-NH₂ and NH₂-PEGn-mTet is added to the activated beads (wherein n is any number, such as 1-100). The ratio between the mPEG3-NH₂ (not available for coupling) and NH₂-PEG24-mTet (available for coupling) is titrated to generate an appropriate density of functional moieties available to attach the polypeptides on the support surface. In certain embodiments, the mean spacing between coupling moieties (e.g., NH₂-PEG₄-mTet) on the solid support is at least 50 nm, at least 100 nm, at least 250 nm, or at least 500 nm. In some specific embodiments, the ratio of NH₂-PEGn-mTet to mPEG3-NH₂ is about or greater than 1:1000, about or greater than 1:10,000, about or greater than 1:100,000, or about or greater than 1:1,000,000. In some further embodiments, the recording tag attaches to the NH₂-PEGn-mTet. In some embodiments, the spacing of the target molecules (e.g., polypeptides) on the support is achieved by controlling the concentration and/or number of available COOH or other functional groups on the support.

### Binding Agent

The methods described herein use a binding agent capable of binding to the target molecules (e.g., polypeptides, polypeptides). The binding reaction may be performed by contacting a single binding agent with a single target, a single binding agent with a plurality of targets, a plurality of binding agents with a single target, or a plurality of binding agents to a plurality of targets. In some embodiments, the plurality of binding agents includes a mixture of binding agents.

A binding agent can be any molecule (e.g., peptide, polypeptide, protein, nucleic acid, carbohydrate, small molecule, and the like) capable of binding to a component or feature of a polypeptide, including a component modified by a functionalizing reagent. A binding agent can be a naturally occurring, synthetically produced, or recombinantly expressed molecule. In some embodiments, the scaffold used to engineer a binding agent can be from any species, e.g., human, non-human, transgenic. A binding agent may bind to a single monomer or subunit of a polypeptide (e.g., a single amino acid) or bind to multiple linked subunits of a polypeptide (e.g., dipeptide, tripeptide, or higher order peptide of a longer polypeptide molecule).

In certain embodiments, a binding agent may be designed to bind covalently. Covalent binding can be designed to be conditional or favored upon binding to the correct moiety. For example, an target and its cognate binding agent may each be modified with a reactive group such that once the target-specific binding agent is bound to the target, a coupling reaction is carried out to create a covalent linkage between the two. Non-specific binding of the binding agent to other locations that lack the cognate reactive group would not result in covalent attachment. In some embodiments, the target comprises a ligand that is capable of forming a covalent bond to a binding agent. In some embodiments, the target comprises a ligand group that is capable of covalent binding to a binding agent. Covalent binding between a binding agent and its target may allow for more stringent washing to be used to remove binding agents that are non-specifically bound, thus increasing the specificity of the assay. The stringency of wash steps may be tuned depending on the affinity of the binding agent to the target and/or the strength and stability of the complex formed.

In some embodiments, the binding reaction involves binding agents configured to provide specificity for binding of the binding agent to the target. A binding agent may bind to an N-terminal peptide, a C-terminal peptide of a polypeptide molecule. A binding agent may bind to an N-terminal amino acid, C-terminal amino acid, or an intervening amino acid of a polypeptide molecule. A binding agent may preferably bind to a chemically modified or labeled amino acid. In certain embodiments, a binding agent may be a selective binding agent. As used herein, selective binding refers to the ability of the binding agent to preferentially bind to a specific ligand (e.g., amino acid or class of amino acids) relative to binding to a different ligand (e.g., amino acid or class of amino acids). Selectivity is commonly referred to as the equilibrium constant for the reaction of displacement of one ligand by another ligand in a complex with a binding agent. Typically, such selectivity is associated with the spatial geometry of the ligand and/or the manner and degree by which the ligand binds to a binding agent, such as by hydrogen bonding, hydrophobic binding, and Van der Waals forces (non-covalent interactions) or by reversible or non-reversible covalent attachment to the binding agent. In one example, a binding agent selectively binds one of the twenty standard amino acids. In some examples, a binding agent binds to an N-terminal amino acid residue, a C-terminal amino acid residue, or an internal amino acid residue. In some embodiments, a binding agent may bind to or is capable of binding to two or more of the twenty standard amino acids. For example, a binding agent may preferentially bind the amino acids A, C, and G over other amino acids. In some other embodiments, the binding agent may selectively or specifically bind more than one amino acid residue. In some embodiments, the binding agent may also have a preference for one or more amino acids at the second, third, fourth, fifth, etc. positions from the terminal amino acid.

In some embodiments, the binding reaction comprises contacting a mixture of binding agents with a mixture of targets and selectively need only be relative to the other binding agents to which the target is exposed. It should also be understood that selectivity of a binding agent need not be absolute to a specific molecule but could be to a portion of a molecule. In some examples, selectivity of a binding agent need not be absolute to a specific amino acid, but could be selective to a class of amino acids, such as amino acids with polar or non-polar side chains, or with electrically (positively or negatively) charged side chains, or with aromatic side chains, or some specific class or size of side chains, and the like. In some embodiments, the ability of a binding agent to selectively bind a feature or component of a polypeptide is characterized by comparing binding abilities of binding agents. For example, the binding ability of a binding agent to the target can be compared to the binding ability of a binding agent which binds to a different target, for example, comparing a binding agent selective for a class of amino acids to a binding agent selective for a different class of amino acids. In some examples, a binding agent selective for non-polar side chains is compared to a binding agent selective for polar side chains. In some embodiments, a binding agent selective for a feature, component of a peptide, or one or more amino acid exhibits at least 1×, at least 2×, at least 5×, at least 10×, at least 50×, at least 100×, or at least 500× more binding compared to a binding agent selective for a different feature, component of a peptide, or one or more amino acid.

In a particular embodiment, the binding agent has a high affinity (binds specifically) and high selectivity for the polypeptide analyte, e.g., polypeptide analyte, of interest. In particular, a high binding affinity with a low off-rate may be efficacious for information transfer between the coding tag and recording tag. In certain embodiments, a binding agent has a Kd of about <500 nM, <200 nM, <100 nM, <50 nM, <10 nM, <5 nM, <1 nM, <0.5 nM, or <0.1 nM. In a particular embodiment, the binding agent is added to the polypeptide at a concentration >1×, >5×, >10×, >100×, or >1000× its Kd to drive binding to completion. For example, binding kinetics of an antibody to a single protein molecule is described in Chang et al., J Immunol Methods (2012) 378 (1-2): 102-115. In a particular embodiment, the provided methods for performing a binding reaction is compatible with a binding agent with medium to low affinity for the target polypeptide.

In some embodiments, a binding agent may bind to a native or unmodified or unlabeled terminal amino acid. Moreover, in some cases, these natural amino acid binders don't recognize N-terminal labels. Directed evolution of aaRS scaffolds can be used to generate higher affinity, higher specificity binding agents that recognized the N-terminal amino acids in the context of an N-terminal label. In another example, Havranak et al. (U.S. Patent Publication No. US 2014/0273004) describes engineering aminoacyl tRNA synthetases (aaRSs) as specific NTAA binders. The amino acid binding pocket of the aaRSs has an intrinsic ability to bind cognate amino acids, but generally exhibits poor binding affinity and specificity. Moreover, these natural amino acid binders don't recognize N-terminal labels. Directed evolution of aaRS scaffolds can be used to generate higher affinity, higher specificity binding agents that recognized the N-terminal amino acids in the context of an N-terminal label.

In certain embodiments, a binding agent may bind to a modified terminal amino acid (e.g., an NTAA that has been modified with a functionalizing reagent). In some embodiments, a binding agent may bind to a chemically or enzymatically modified terminal amino acid. A modified or labeled NTAA can be one that is functionalized with phenylisothiocyanate, PITC, 1-fluoro-2,4-dinitrobenzene (Sanger's reagent, DNFB), benzyloxycarbonyl chloride or carbobenzoxy chloride (Cbz-Cl), N-(Benzyloxycarbonyloxy) succinimide (Cbz-OSu or Cbz-O-NHS), dansyl chloride (DNS-Cl, or 1-dimethylaminonaphthalene-5-sulfonyl chloride), 4-sulfonyl-2-nitrofluorobenzene (SNFB), N-Acetyl-Isatoic Anhydride, Isatoic Anhydride, a guanidinylation reagent, a thioacetylation reagent, or a thiobenzylation reagent, or a diheterocyclic methanimine reagent.

Functionalizing reagent can be chosen to increase affinity, specificity and/or selectivity of binding agents towards particular terminal amino acid residues. Examples of such functionalizing reagents and binding agents that can bind to NTAA or functionalized NTAA with certain levels of specificity and selectivity are disclosed in the following patent publications, incorporated herein by reference: U.S. Pat. No. 9,435,810 B2, US 2019/0145982 A1, US 2020/0348308 A1. In some embodiments, binding agents configured to bind to functionalized NTAA residues of polypeptides can be developed through directed evolution of scaffolds with 10 μM or lower affinity using phage display, as disclosed in WO 2022/072560 A1, and in US patent publication US 2022/0283175 A1, incorporated herein by reference. In some specific embodiments, the engineered metalloprotein binding agents comprise an amino acid sequence having at least about 80%, 90% or 95% sequence homology to any one of the amino acid sequences set forth in SEQ ID NO: 14 and SEQ ID NO: 16-SEQ ID NO: 18 (see also US patent publication US 2022/0283175 A1, incorporated herein by reference).

In some preferred embodiments, the N-terminal functionalizing reagent that is used to modify polypeptide analytes is selected from the group consisting of compounds of the following formula:

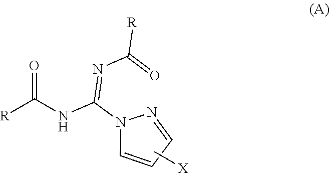

wherein R is CH₃, CF₃, OC(CH₃)₃, or OCH₂C₆H₅,
and X is H, CH₃, CF₃, CF₂H, or OCH₃;

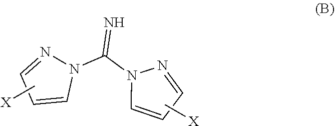

wherein X is H, CH₃, CF₃, CF₂H, OCH₃, or SO₂NH₂;

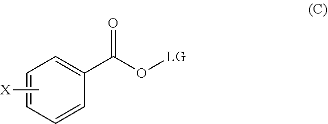

wherein X is H, F, Cl, OCH₃, OCF₃, CN, or SO₂NH₂,
and LG is succinimide, pentafluorophenyl, or tetrafluorophenyl;
and

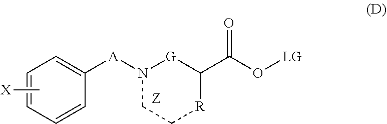

wherein X is H, F, Cl, NH₂, OCH₃, OCF₃, CN, or SO₂NH₂, A=CONH or SO₂, G=0 or 1 CH₂, R is any amino acid or unnatural amino acid, and Z ring=0 (not there), 1, 2, or 3 CH₂.

In some embodiments, the engineered binding agent binds to the N-terminally functionalized target peptide with a thermodynamic dissociation constant (Kd) of 200 nM or less. In some preferred embodiments, the engineered binding agent binds to the N-terminally functionalized target peptide with a thermodynamic dissociation constant (Kd) of 100 nM or less.

In certain embodiments, a binding agent can be an aptamer (e.g., peptide aptamer, DNA aptamer, or RNA aptamer), a peptoid, an antibody or a specific binding fragment thereof, an amino acid binding protein or enzyme, an antibody binding fragment, an antibody mimetic, a protein, a peptidomimetic. Detailed descriptions of antibody and/or protein engineering, including relevant protocols, can be found in, among other places, J. Maynard and G. Georgiou, 2000, Ann. Rev. Biomed. Eng. 2:339-76; Antibody Engineering, R. Kontermann and S. Dubel, eds., Springer Lab Manual, Springer Verlag (2001); U.S. Pat. No. 5,831,012; and S. Paul, Antibody Engineering Protocols, Humana Press (1995). As with antibodies, nucleic acid and peptide aptamers that specifically recognize a polypeptide, e.g., polypeptide, can be produced using known methods. Aptamers bind target molecules in a highly specific, conformation-dependent manner, typically with very high affinity, although aptamers with lower binding affinity can be selected if desired. Aptamers have been shown to distinguish between targets based on very small structural differences such as the presence or absence of a methyl or hydroxyl group and certain aptamers can distinguish between D- and L-enantiomers. Aptamers have been shown to retain functional activity after biotinylation, fluorescein labeling, and when attached to glass surfaces and microspheres. (see, e.g., Jayasena, 1999, Clin Chem 45:1628-50; Kusser2000, J. Biotechnol. 74:27-39; Colas, 2000, Curr Opin Chem Biol 4:54-9). Aptamers which specifically bind arginine and AMP have been described as well (see, Patel and Suri, 2000, J. Biotech. 74:39-60). Oligonucleotide aptamers that bind to a specific amino acid have been disclosed in Gold et al. (1995, Ann. Rev. Biochem. 64:763-97). RNA aptamers that bind amino acids have also been described (Ames and Breaker, 2011, RNA Biol. 8; 82-89; Mannironi et al., 2000, RNA 6:520-27; Famulok, 1994, J. Am. Chem. Soc. 116:1698-1706).

A binding agent can be made by modifying naturally-occurring or synthetically-produced proteins by genetic engineering to introduce one or more mutations in the amino acid sequence to produce engineered proteins that bind to a specific component or feature of a polypeptide (e.g., NTAA, CTAA, or post-translationally modified amino acid or a peptide). In some embodiments, a binding agent that selectively binds to a labeled or functionalized NTAA can be utilized. For example, exopeptidases (e.g., aminopeptidases, carboxypeptidases), exoproteases, mutated exoproteases, mutated anticalins, mutated ClpSs, antibodies, or tRNA synthetases can be modified to create a binding agent that selectively binds to a particular NTAA. In another example, carboxypeptidases can be modified to create a binding agent that selectively binds to a particular CTAA. Strategies for directed evolution of proteins are known in the art (e.g., Yuan et al., 2005, Microbiol. Mol. Biol. Rev. 69:373-392), and include phage display, ribosomal display, mRNA display, CIS display, CAD display, emulsions, cell surface display method, yeast surface display, bacterial surface display, etc.

In yet another embodiment, a binding agent may be a modified aminopeptidase. In some embodiments, the binding agent may be a modified aminopeptidase that has been engineered to recognize the DNP-labeled NTAA providing cyclic control of aminopeptidase degradation of the peptide. Once the DNP-labeled NTAA is eliminated, another cycle of DNFB derivatization is performed in order to bind and eliminate the newly exposed NTAA. In preferred particular embodiment, the aminopeptidase is a monomeric metallo-protease, such an aminopeptidase activated by zinc (Calcagno et al., Appl Microbiol Biotechnol. (2016) 100 (16): 7091-7102). In another example, a binding agent may selectively bind to an NTAA that is modified with sulfonyl nitrophenol (SNP), e.g., by using 4-sulfonyl-2-nitrofluorobenzene (SNFB). Other reagents that may be used to functionalize the NTAA include trifluoroethyl isothiocyanate, allyl isothiocyanate, and dimethylaminoazobenzene isothiocyanate, or a reagent as described in International Patent Publication No. WO 2019/089846. A binding agent may be engineered for high affinity for a modified NTAA, high specificity for a modified NTAA, or both.

In certain embodiments, the binding agent further comprises one or more detectable labels such as fluorescent labels, in addition to the binding moiety. In some embodiments, the binding agent does not comprise a polynucleotide such as a coding tag. Optionally, the binding agent comprises a synthetic or natural antibody. In some embodiments, the binding agent comprises an aptamer. In one embodiment, the detectable label is optically detectable. In some embodiments, the detectable label comprises a fluorescently moiety, a color-coded nanoparticle, a quantum dot or any combination thereof. In one embodiment the label comprises a polystyrene dye encompassing a core dye molecule such as a FluoSphere™, Nile Red, fluorescein, rhodamine, derivatized rhodamine dyes, such as TAMRA, phosphor, polymethadine dye, fluorescent phosphoramidite, TEXAS RED, green fluorescent protein, acridine, cyanine, cyanine 5 dye, cyanine 3 dye, 5-(2′-aminoethyl)-aminonaphthalene-1-sulfonic acid (EDANS), BODIPY, 120 ALEXA or a derivative or modification of any of the foregoing. In one embodiment, the detectable label is resistant to photobleaching while producing lots of signal (such as photons) at a unique and easily detectable wavelength, with high signal-to-noise ratio.

In a particular embodiment, anticalins are engineered for both high affinity and high specificity to labeled NTAAs (e.g., PTC, modified-PTC, Cbz, DNP, SNP, acetyl, guanidinyl, amino guanidinyl, heterocyclic methanimine, etc.). Certain varieties of anticalin scaffolds have suitable shape for binding single amino acids, by virtue of their beta barrel structure. An N-terminal amino acid (either with or without modification) can potentially fit and be recognized in this “beta barrel” bucket. High affinity anticalins with engineered novel binding activities have been described (reviewed by Skerra, 2008, FEBS J. 275:2677-2683). For example, anticalins with high affinity binding (low nM) to fluorescein and digoxygenin have been engineered (Gebauer et al., 2012, Methods Enzymol 503:157-188). Engineering of alternative scaffolds for new binding functions has also been reviewed by Banta et al. (2013, Annu. Rev. Biomed. Eng. 15:93-113).

In some embodiments, the binding agent is derived from a biological, naturally occurring, non-naturally occurring, or synthetic source. In some examples, the binding agent is derived from de novo protein design (Huang et al., (2016) 537 (7620): 320-327).

In certain embodiments, a polypeptide, e.g., a protein, is also contacted with a non-cognate binding agent. As used herein, a non-cognate binding agent is referring to a binding agent that is selective for a different target (e.g., polypeptide feature or component) than the particular target being considered. For example, if the n NTAA is phenylalanine, and the peptide is contacted with three binding agents selective for phenylalanine, tyrosine, and asparagine, respectively, the binding agent selective for phenylalanine would be first binding agent capable of selectively binding to the nth NTAA (i.e., phenylalanine), while the other two binding agents would be non-cognate binding agents for that peptide (since they are selective for NTAAs other than phenylalanine). The tyrosine and asparagine binding agents may, however, be cognate binding agents for other peptides in the sample. If the n NTAA (phenylalanine) was then cleaved from the peptide, thereby converting the n−1 amino acid of the peptide to the n−1 NTAA (e.g., tyrosine), and the peptide was then contacted with the same three binding agents, the binding agent selective for tyrosine would be second binding agent capable of selectively binding to the n−1 NTAA (i.e., tyrosine), while the other two binding agents would be non-cognate binding agents (since they are selective for NTAAs other than tyrosine).

Thus, it should be understood that whether an agent is a binding agent or a non-cognate binding agent will depend on the nature of the particular polypeptide feature or component currently available for binding. Also, if multiple polypeptides are analyzed in a multiplexed reaction, a binding agent for one polypeptide may be a non-cognate binding agent for another, and vice versa. According, it should be understood that the following description concerning binding agents is applicable to any type of binding agent described herein (i.e., both cognate and non-cognate binding agents).

In certain embodiments, the concentration of the binding agents in a solution is controlled to reduce background and/or false positive results of the assay. In some embodiments, the concentration of a binding agent can be at any suitable concentration, e.g., at about 0.0001 nM, about 0.001 nM, about 0.01 nM, about 0.1 nM, about 1 nM, about 5 nM, about 10 nM, about 50 nM, about 100 nM, about 500 nM, or about 1,000 nM. In some embodiments, the ratio between the soluble binding agent molecules and the immobilized polypeptide, e.g., polypeptides, can be at any suitable range, e.g., at about 0.00001:1, about 0.0001:1, about 0.001:1, about 0.01:1, about 0.1:1, about 1:1, about 2:1, about 5:1, about 10:1, about 50:1, about 100:1, about 104:1, about 105:1, about 106:1, or higher, or any ratio in between the above listed ratios. Higher ratios between the soluble binding agent molecules and the immobilized polypeptide(s) and/or the nucleic acids can be used to drive the binding and/or the coding tag information transfer to completion. This may be particularly useful for detecting and/or analyzing low abundance polypeptides in a sample.

### Methods of Assaying Polypeptides

In some aspects, the polypeptide analysis includes contacting the polypeptide with a binding agent capable of binding to the polypeptide, wherein the binding agent comprises a coding tag with identifying information regarding the binding agent; and transferring the information of the coding tag to a recording tag (associated with the target polypeptide) to generate an extended recording tag. In some further embodiments, transferring the information of the coding tag to the recording tag to extend the recording tag may be repeated one or more times. In some embodiments, the polypeptide analysis is a next generation protein assay (NGPA) using multiple binding agents and enzymatically-mediated sequential information transfer. In some cases, the assay is performed on immobilized target molecules bound a cognate binding agent (e.g., antibody) and forming a stable complex, then transferring information from the coding tags of bound antibodies to the recording tag associated with the target. In some cases, the assay is performed on immobilized target molecules bound by two or more cognate binding agents (e.g., antibodies). After a cognate antibody binding event, a combined primer extension and DNA nicking step is used to transfer information from the coding tags of bound antibodies to the recording tag. In some cases, polyclonal antibodies (or mixed population of monoclonal antibody) to multivalent epitopes on a protein can be used for the assay (see e.g., US 2019/0145982 A1 incorporated herein by reference). In some embodiments, the sequence (or a portion of the sequence thereof) and/or the identity of a target protein is determined using polypeptide analysis. In some examples, the polypeptide analysis includes assessing at least a partial sequence or identity of the polypeptide using suitable techniques or procedures. For example, at least a partial sequence of the polypeptide can be assessed by N-terminal amino acid analysis or C-terminal amino acid analysis. In some embodiments, at least a partial sequence of the polypeptide can be assessed using a ProteoCode™ assay. In some examples, at least a partial sequence of the polypeptide can be assessed by the techniques or procedures disclosed and/or claimed in the published applications US 2019/0145982 A1, US 2020/0348308 A1 and US 2020/0348307 A1. In some embodiments, the method includes treating the target peptide with a reagent for modifying a terminal amino acid of the peptide. In some embodiments, the target peptide is contacted with the reagent for modifying (e.g., functionalizing) a terminal amino acid before removing the terminal amino acid. In some embodiments, the method further includes removing the binding agent after transferring information from the coding tag to the recording tag. In some aspects, removing the binding agent is performed after transferring information from the coding tag associated with the binding agent to the recording tag associated with the target. In some embodiments, the method further comprises transferring information of a coding tag associated with the binding agent to the recording tag associated with the target to generate an extended recording tag, thereby generating an extended recording tag. Methods of transferring information of a coding tag associated with the binding agent to the recording tag are disclosed in applications US 2019/0145982 A1, US 2020/0348308 A1, US 2020/0348307 A1, the content of which are incorporated herein by reference.

The polypeptide analysis may include one or more cycles of binding with additional binding agents to the terminal amino acid, transferring information from the additional binding agents to the extended nucleic acid thereby generating a higher order or a further extended recording tag containing information from two or more coding tags, and eliminating the terminal amino acid in a cyclic manner. Additional binding, transfer, labeling, and removal, can occur as described above up to n amino acids to generate an nth order extended nucleic acid, which collectively represent the peptide. In some embodiments, steps including the NTAA in the described exemplary approach can be performed instead with a C terminal amino acid (CTAA). In some embodiments, the order of the steps in the process for a degradation-based peptide or polypeptide sequencing assay can be reversed or be performed in various orders. For example, in some embodiments, the terminal amino acid labeling can be conducted before and/or after the polypeptide is bound to the binding agent. In some embodiments, contacting of the first binding agent and second binding agent to the target, and optionally any further binding agents (e.g., third binding agent, fourth binding agent, fifth binding agent, and so on), are performed at the same time. In an example, the first binding agent and second binding agent, and optionally any further order binding agents, can be first pooled together and added to the polypeptide, or can be added simultaneously to the polypeptide without prior pooling. In other embodiments, the first binding agent and second binding agent, and optionally any further order binding agents, are each contacted with the polypeptide in separate binding cycles, added in sequential order. In certain embodiments, multiple binding agents are used at the same time in parallel. This parallel approach saves time and reduces non-specific binding by non-cognate binding agents to a site that is bound by a cognate binding agent (because the binding agents are in competition).

The extended nucleic acid (e.g., recording tag) is any nucleic acid molecule or sequenceable polymer molecule (see, e.g., Niu et al., 2013, Nat. Chem. 5:282-292; Roy et al., 2015, Nat. Commun. 6:7237) that comprises identifying information for a polypeptide, e.g., a protein. An extended nucleic acid associated with the polypeptide, e.g., protein, with identifying information from the coding tag may comprise information from a binding agent's coding tag representing each binding cycle performed. However, in some cases, an extended nucleic acid may also experience a “missed” binding cycle, e.g., if a binding agent fails to bind to the polypeptide, because the coding tag was missing, damaged, or defective, because the primer extension reaction failed. Even if a binding event occurs, transfer of information from the coding tag may be incomplete or less than 100% accurate, e.g., because a coding tag was damaged or defective, because errors were introduced in the primer extension reaction). Thus, an extended nucleic acid may represent 100%, or up to 95%, 90%, 75%, 50%, 30%, or any subrange thereof, of binding events that have occurred on its associated polypeptide. Moreover, the coding tag information present in the extended nucleic acid may have at least 30%, 50%, 75%, 90%, 95%, or 100% identity the corresponding coding tags. In certain embodiments, an extended recording tag associated with the immobilized peptide may comprise information from multiple coding tags representing multiple, successive binding events. In these embodiments, a single, concatenated extended recording tag associated with the immobilized peptide can be representative of a single polypeptide. As referred to herein, transfer of coding tag information to the recording tag associated with the immobilized peptide also includes transfer to an extended recording tag as would occur in methods involving multiple, successive binding events.

Coding tag information associated with a specific binding agent may be transferred to a recording tag using a variety of methods. In certain embodiments, information of a coding tag is transferred to a recording tag via primer extension (Chan et al. (2015) Curr Opin Chem Biol 26:55-61). A spacer sequence on the 3′-terminus of a recording tag or an extended recording tag anneals with complementary spacer sequence on the 3′ terminus of a coding tag and a polymerase (e.g., strand-displacing polymerase) extends the recording tag sequence, using the annealed coding tag as a template. In some embodiments, oligonucleotides complementary to coding tag encoder sequence and 5′ spacer can be pre-annealed to the coding tags to prevent hybridization of the coding tag to internal encoder and spacer sequences present in an extended recording tag. The 3′ terminal spacer, on the coding tag, remaining single stranded, preferably binds to the terminal 3′ spacer on the recording tag. In other embodiments, a nascent recording tag can be coated with a single stranded binding protein to prevent annealing of the coding tag to internal sites.

In some embodiments, a DNA polymerase that is used for primer extension possesses strand-displacement activity and has limited or is devoid of 3′-5 exonuclease activity. Several of many examples of such polymerases include Klenow exo-(Klenow fragment of DNA Pol 1), T4 DNA polymerase exo-, T7 DNA polymerase exo (Sequenase 2.0), Pfu exo-, Vent exo-, Deep Vent exo-, Bst DNA polymerase large fragment exo-, Bca Pol, 9°N Pol, and Phi29 Pol exo-. In a preferred embodiment, the DNA polymerase is active at room temperature and up to 45° C. In another embodiment, a “warm start” version of a thermophilic polymerase is employed such that the polymerase is activated and is used at about 40° C.-50° C. An exemplary warm start polymerase is Bst 2.0 Warm Start DNA Polymerase (New England Biolabs).

Mis-priming or self-priming events, such as when the terminal spacer sequence of the recording tag primes extension self-extension may be minimized by inclusion of single stranded binding proteins (T4 gene 32, E. coli SSB, etc.), DMSO (1-10%), formamide (1-10%), BSA (10-100 μg/ml), TMACl (1-5 mM), ammonium sulfate (10-50 mM), betaine (1-3 M), glycerol (5-40%), or ethylene glycol (5-40%), in the primer extension reaction.

Most type A polymerases are devoid of 3′ exonuclease activity (endogenous or engineered removal), such as Klenow exo-, T7 DNA polymerase exo-(Sequenase 2.0), and Taq polymerase catalyzes non-templated addition of a nucleotide, preferably an adenosine base (to lesser degree a G base, dependent on sequence context) to the 3′ blunt end of a duplex amplification product. For Taq polymerase, a 3′ pyrimidine (C>T) minimizes non-templated adenosine addition, whereas a 3′ purine nucleotide (G>A) favours non-templated adenosine addition. In some embodiments, using Taq polymerase for primer extension, placement of a thymidine base in the coding tag between the spacer sequence distal from the binding agent and the adjacent barcode sequence (e.g., encoder sequence or cycle specific sequence) accommodates the sporadic inclusion of a non-templated adenosine nucleotide on the 3′ terminus of the spacer sequence of the recording tag. In this manner, the extended recording tag associated with the immobilized peptide (with or without a non-templated adenosine base) can anneal to the coding tag and undergo primer extension.

In some embodiments, polymerase extension buffers are comprised of 40-120 mM buffering agent such as Tris-Acetate, Tris-HCl or HEPES at a pH of 6-9.

In some embodiments, to minimize non-specific interaction of the coding tag labeled binding agents in solution with the nucleic acids of immobilized proteins, competitor (also referred to as blocking) oligonucleotides complementary to nucleic acids containing spacer sequences (e.g., on the recording tag) can be added to binding reactions to minimize non-specific interactions. In some embodiments, the blocking oligonucleotides contain a sequence that is complementary to the coding tag or a portion thereof attached to the binding agent. In some embodiments, blocking oligonucleotides are relatively short. Excess competitor oligonucleotides are washed from the binding reaction prior to primer extension, which effectively dissociates the annealed competitor oligonucleotides from the nucleic acids on the recording tag, especially when exposed to slightly elevated temperatures (e.g., 30-50° C.). Blocking oligonucleotides may comprise a terminator nucleotide at its 3′ end to prevent primer extension.

In some embodiments, the transfer of identifying information (e.g., from a coding tag to a recording tag) can be accomplished by ligation (e.g., an enzymatic or chemical ligation, a splint ligation, a sticky end ligation, a single-strand (ss) ligation such as a ssDNA ligation, or any combination thereof), a polymerase-mediated reaction (e.g., primer extension of single-stranded nucleic acid or double-stranded nucleic acid), or any combination thereof. Examples of ligases include, but are not limited to CV DNA ligase, T4 DNA ligase, T7 DNA ligase, T3 DNA ligase, Taq DNA ligase, E. coli DNA ligase, 9°N DNA ligase, Electroligase® (See e.g., U.S. Patent Publication No. US20140378315). Alternatively, a ligation may be a chemical ligation reaction, such as chemical ligation using standard chemical ligation or “click chemistry” (Gunderson et al., Genome Res (1998) 8 (11): 1142-1153; El-Sagheer et al., Proc Natl Acad Sci USA (2011) 108 (28): 11338-11343; Sharma et al., Anal Chem (2012) 84 (14): 6104-6109; Roloff et al., Methods Mol Biol (2014) 1050:131-141).

In another embodiment, transfer of PNAs can be accomplished with chemical ligation using published techniques. The structure of PNA is such that it has a 5′ N-terminal amine group and an unreactive 3′ C-terminal amide. Chemical ligation of PNA requires that the termini be modified to be chemically active. This is typically done by derivatizing the 5′ N-terminus with a cysteinyl moiety and the 3′ C-terminus with a thioester moiety. Such modified PNAs easily couple using standard native chemical ligation conditions (Roloff et al., (2013) Bioorgan. Med. Chem. 21:3458-3464).

In some embodiments, coding tag information can be transferred using topoisomerase. Topoisomerase can be used be used to ligate a topo-charged 3′ phosphate on the recording tag (or extensions thereof or any nucleic acids attached) to the 5′ end of the coding tag, or complement thereof (Shuman et al., 1994, J. Biol. Chem. 269:32678-32684).

In some examples, the final extended recording tag containing information from one or more binding agents is optionally flanked by universal priming sites to facilitate downstream amplification and/or DNA sequencing. The forward universal priming site (e.g., Illumina's P5-S1 sequence) can be part of the original design of the recording tag and the reverse universal priming site (e.g., Illumina's P7-S2′ sequence) can be added as a final step in the extension of the nucleic acid. In some embodiments, the addition of forward and reverse priming sites can be done independently of a binding agent.

### Recording Tag and Immobilization Methods

In some embodiments, the target polypeptide (e.g., protein or polypeptide) may be labeled with a nucleic acid molecule or a oligonucleotide (e.g., recording tag). In some aspects, a plurality of target polypeptides in the sample is provided with recording tags. The recording tags may be associated or attached, directly or indirectly to the target polypeptides using any suitable means. In some embodiments, a polypeptide may be associated with one or more recording tags. In some aspects, the recording tag may be any suitable sequenceable moiety to which identifying information can be transferred (e.g., information from one or more coding tags). In some embodiments, at least one recording tag is associated or co-localized directly or indirectly with the target polypeptide (e.g., polypeptide). In another embodiment, multiple recording tags are attached to the polypeptide, such as to the lysine residues or peptide backbone. In some embodiments, a polypeptide labeled with multiple recording tags is fragmented or digested into smaller peptides, with each peptide labeled on average with one recording tag. A recording tag may comprise DNA, RNA, or polynucleotide analogs including PNA, gPNA, GNA, HNA, BNA, XNA, TNA, or a combination thereof. A recording tag may be single stranded, or partially or completely double stranded. A recording tag may have a blunt end or overhanging end. In other embodiments, a subset of polypeptides within a sample are labeled with recording tags. In some embodiments, the recording tag may comprise a unique molecular identifier, a compartment tag, a partition barcode, sample barcode, a fraction barcode, a spacer sequence, a universal priming site, or any combination thereof. In some embodiments, the recording tag may comprise a blocking group, such as at the 3′-terminus of the recording tag. In some cases, the 3′-terminus of the recording tag is blocked to prevent extension of the recording tag by a polymerase.

In some embodiments, the recording tag can include a sample identifying barcode. A sample barcode is useful in the multiplexed analysis of a set of samples in a single reaction vessel or immobilized to a single solid support (e.g., a bead or a planar substrate) or collection of solid supports. For example, polypeptides from many different samples can be labeled with recording tags with sample-specific barcodes, and then all the samples pooled together prior to immobilization to a support, cyclic binding of the binding agent, and recording tag analysis.

In certain embodiments, a recording tag comprises an optional unique molecular identifier (UMI), which provides a unique identifier tag for each polypeptides (e.g., polypeptide) to which the UMI is associated with. A UMI can be about 3 to about 40 bases, about 3 to about 20 bases, or about 3 to about 10 bases, or about 3 to about 8 bases in length. A UMI can be used to de-convolute sequencing data from a plurality of extended recording tags to identify sequence reads from individual polypeptides. In some embodiments, within a library of polypeptides, each polypeptide is associated with a single recording tag, with each recording tag comprising a unique UMI. In other embodiments, multiple copies of a recording tag are associated with a single polypeptide, with each copy of the recording tag comprising the same UMI.

In certain embodiments, a recording tag comprises a universal priming site, e.g., a forward or 5′ universal priming site. A universal priming site is a nucleic acid sequence that may be used for priming a library amplification reaction and/or for sequencing. A universal priming site may include, but is not limited to, a priming site for PCR amplification, flow cell adaptor sequences that anneal to complementary oligonucleotides on flow cell surfaces (e.g., Illumina next generation sequencing), a sequencing priming site, or a combination thereof. A universal priming site can be about 10 bases to about 60 bases. In some embodiments, a universal priming site comprises an Illumina P5 primer (5′-AATGATACGGCGACCACCGA-3′-SEQ ID NO:1) or an Illumina P7 primer (5′-CAAGCAGAAGACGGCATACGAGAT-3′-SEQ ID NO:2).

In certain embodiments, a recording tag comprises a compartment tag. In some embodiments, the compartment tag is a component within a recording tag. In some embodiments, the recording tag can also include a barcode which represents a compartment tag in which a compartment, such as a droplet, microwell, physical region on a support, etc. is assigned a unique barcode. The association of a compartment with a specific barcode can be achieved in any number of ways such as by encapsulating a single barcoded bead in a compartment, e.g., by direct merging or adding a barcoded droplet to a compartment, by directly printing or injecting a barcode reagents to a compartment, etc. Applied to protein molecules partitioned into compartments, compartment-specific barcodes can be used to map analyzed peptides back to their originating protein molecules in the compartment.

In certain embodiments, a polypeptide can be immobilized to a support by an affinity capture reagent (and optionally covalently crosslinked), wherein the recording tag is associated with the affinity capture reagent directly, or alternatively, the polypeptide can be directly immobilized to the support with a recording tag. In one embodiment, the polypeptide is attached to a bait nucleic acid which hybridizes to a capture nucleic acid and is ligated to a capture nucleic acid which comprises a reactive coupling moiety for attaching to the support (see, e.g., US 2022/0049246 A1, incorporated by reference herein). In some examples, the bait or capture nucleic acid may serve as a recording tag to which information regarding the polypeptide can be transferred. In some embodiments, the polypeptide is attached to a bait nucleic acid to form a nucleic acid-polypeptide chimera. In some embodiments, the immobilization methods comprise bringing the nucleic acid-polypeptide chimera into proximity with a support by hybridizing the bait nucleic acid to a capture nucleic acid attached to the support, and covalently coupling the nucleic acid-polypeptide chimera to the solid support. In some cases, the nucleic acid-polypeptide chimera is coupled indirectly to the solid support, such as via a linker. In some embodiments, a plurality of the nucleic acid-polypeptide chimeras is coupled on the solid support and any adjacently coupled nucleic acid-polypeptide chimeras are spaced apart from each other at an average distance of about 50 nm or greater.

In some embodiments, the density or number of polypeptides provided with a recording tag is controlled or titrated. In some examples, the desired spacing, density, and/or amount of recording tags in the sample may be titrated by providing a diluted or controlled number of recording tags. In some examples, the desired spacing, density, and/or amount of recording tags may be achieved by spiking a competitor or “dummy” competitor molecule when providing, associating, and/or attaching the recording tags. In some cases, the “dummy” competitor molecule reacts in the same way as a recording tag being associated or attached to a polypeptide in the sample but the competitor molecule does not function as a recording tag. In some specific examples, if a desired density is 1 functional recording tag per 1,000 available sites for attachment in the sample, then spiking in 1 functional recording tag for every 1,000 “dummy” competitor molecules is used to achieve the desired spacing. In some examples, the ratio of functional recording tags is adjusted based on the reaction rate of the functional recording tags compared to the reaction rate of the competitor molecules.

In some examples, the labeling of the polypeptide with a recording tag is performed using standard amine coupling chemistries. In a particular embodiment, the recording tag can comprise a reactive moiety (e.g., for conjugation to a solid support, a multifunctional linker, or a polypeptide), a linker, a universal priming sequence, a barcode, an optional UMI, and a spacer (Sp) sequence for facilitating information transfer to/from a coding tag. In another embodiment, the protein is labeled with a universal DNA tag prior to proteinase digestion into peptides. The universal DNA tags on the labeled peptides from the digest can then be converted into an informative and effective recording tag. A universal DNA tag comprises a short sequence of nucleotides that are used to label a polypeptide and can be used as point of attachment. For example, a recording tag may comprise at its terminus a sequence complementary to the universal DNA tag. In certain embodiments, a universal DNA tag is a universal priming sequence. Upon hybridization of the universal DNA tags on the labeled protein to complementary sequence in recording tags (e.g., bound to beads), the annealed universal DNA tag may be extended via primer extension, transferring the recording tag information to the DNA tagged protein.

The recording tags may comprise a reactive moiety for a cognate reactive moiety present on the target polypeptide, e.g., the target protein, (e.g., click chemistry labeling, photoaffinity labeling). For example, recording tags may comprise an azide moiety for interacting with alkyne-derivatized proteins, or recording tags may comprise a benzophenone for interacting with native proteins, etc. Upon binding of the target protein by the target protein specific binding agent, the recording tag and target protein are coupled via their corresponding reactive moieties. After the target protein is labeled with the recording tag, the target-protein specific binding agent may be removed by digestion of the DNA capture probe linked to the target-protein specific binding agent. For example, the DNA capture probe may be designed to contain uracil bases, which are then targeted for digestion with a uracil-specific excision reagent (e.g., USER™), and the target-protein specific binding agent may be dissociated from the target protein. In some embodiments, other types of linkages besides hybridization can be used to link the recording tag to a polypeptide. A suitable linker can be attached to various positions of the recording tag, such as the 3′ end, at an internal position, or within the linker attached to the 5′ end of the recording tag.

### Coding Tag

The coding tag associated with the binding agent is or comprises a polynucleotide with any suitable length, e.g., a nucleic acid molecule of about 3 bases to about 100 bases, that comprises identifying information for its associated binding agent. A coding tag may be composed of DNA, RNA, polynucleotide analogs, or a combination thereof. Polynucleotide analogs include PNA, gPNA, BNA, GNA, TNA, LNA, morpholino polynucleotides, 2′-O-Methyl polynucleotides, alkyl ribosyl substituted polynucleotides, phosphorothioate polynucleotides, and 7-deaza purine analogs. A coding tag may also be made from a “sequenceable polymer” (see, e.g., Niu et al., 2013, Nat. Chem. 5:282-292; Roy et al., 2015, Nat. Commun. 6:7237; Lutz, 2015, Polypeptides 48:4759-4767). A coding tag may comprise an encoder sequence or a sequence with identifying information, which is optionally flanked by one spacer on one side or optionally flanked by a spacer on each side. A coding tag may also be comprised of an optional UMI and/or an optional binding cycle-specific barcode. A coding tag may refer to the coding tag that is directly attached to a binding agent, or to a complementary sequence hybridized to the coding tag directly attached to a binding agent (e.g., for double stranded coding tags). In certain embodiments, a coding tag may further comprise a binding cycle specific spacer or barcode, a unique molecular identifier, a universal priming site, or any combination thereof. A coding tag may be a single stranded molecule, a double stranded molecule, or a partially double stranded. A coding tag may comprise blunt ends, overhanging ends, or one of each. In some embodiments, the coding tag may comprise a hairpin. In certain embodiments, the hairpin comprises mutually complementary nucleic acid regions are connected through a nucleic acid strand. In some embodiments, the nucleic acid hairpin can also further comprise 3′ and/or 5′ single-stranded region(s) extending from the double-stranded stem segment. In some examples, the hairpin comprises a single strand of nucleic acid.

In some embodiments, each unique binding agent within a library of binding agents has a unique encoder sequence (a barcode sequence that identifies the associated binding agent). For example, 20 unique encoder sequences may be used for a library of 20 binding agents that bind to the 20 standard amino acids. In another example, 30 unique encoder sequences may be used for a library of 30 binding agents that bind to the 20 standard amino acids and 10 post-translational modified amino acids (e.g., phosphorylated amino acids, acetylated amino acids, methylated amino acids). In other embodiments, two or more different binding agents may share the same encoder sequence. For example, two binding agents that each bind to a different standard amino acid may share the same encoder sequence.

In certain embodiments, a coding tag further comprises a spacer sequence at one end or both ends. In certain embodiments, the spacer is binding agent-specific so that a spacer from a previous binding cycle only interacts with a spacer from the appropriate binding agent in a current binding cycle. An example would be pairs of cognate antibodies containing spacer sequences that only allow information transfer if both antibodies sequentially bind to the polypeptide. A spacer sequence may be used as the primer annealing site for a primer extension reaction, or a splint or sticky end in a ligation reaction. In other embodiments, the coding tags within a library of binding agents do not have a binding cycle-specific spacer sequence. A cycle-specific spacer sequence can also be used to concatenate information of coding tags onto a single recording tag when a population of recording tags is associated with a polypeptide. The first binding cycle transfers information from the coding tag to a randomly-chosen recording tag, and subsequent binding cycles can prime only the extended recording tag using cycle-dependent spacer sequences.

A coding tag may include a terminator nucleotide incorporated at the 3′ end of the 3′ spacer sequence. After a binding agent binds to a polypeptide and their corresponding coding tag and recording tags anneal via complementary spacer sequences, it is possible for primer extension to transfer information from the coding tag to the recording tag, or to transfer information from the recording tag to the coding tag. Addition of a terminator nucleotide on the 3′ end of the coding tag prevents transfer of recording tag information to the coding tag. It is understood that for embodiments described herein involving generation of extended coding tags, it may be preferable to include a terminator nucleotide at the 3′ end of the recording tag to prevent transfer of coding tag information to the recording tag.

In some embodiments, the coding tag sequence can be optimized for the particular sequencing analysis platform. In a particular embodiment, the sequencing platform is nanopore sequencing. In some embodiments, the sequencing platform has a per base error rate of >1%, >5%, >10%, >15%, >20%, >25%, or >30%. For example, if the extended nucleic acid is to be analyzed using a nanopore sequencing instrument, the barcode sequences (e.g., sequences comprising identifying information from the coding tag) can be designed to be optimally electrically distinguishable in transit through a nanopore.

A coding tag can be joined to a binding agent directly or indirectly, by any means known in the art, including covalent and non-covalent interactions. In some embodiments, a coding tag may be joined to binding agent enzymatically or chemically. In some embodiments, a coding tag may be joined to a binding agent via ligation. In some cases, a coding tag may be joined to a binding agent to an unnatural amino acid, such as via a covalent interaction with an unnatural amino acid. In some embodiments, a binding agent is joined to a coding tag via SpyCatcher-SpyTag interaction. The SpyTag peptide forms an irreversible covalent bond to the SpyCatcher protein via a spontaneous isopeptide linkage, thereby offering a genetically encoded way to create peptide interactions that resist force and harsh conditions (Zakeri et al., 2012, Proc. Natl. Acad. Sci. 109:E690-697; Li et al., 2014, J. Mol. Biol. 426:309-317). A binding agent may be expressed as a fusion protein comprising the SpyCatcher protein.

In other embodiments, a binding agent is joined to a coding tag via SnoopTag-SnoopCatcher peptide-protein interaction. The SnoopTag peptide forms an isopeptide bond with the SnoopCatcher protein (Veggiani et al., Proc. Natl. Acad. Sci. USA, 2016, 113:1202-1207). A binding agent may be expressed as a fusion protein comprising the SnoopCatcher protein. In yet other embodiments, a binding agent is joined to a coding tag via the HaloTag® protein fusion tag and its chemical ligand. HaloTag is a modified haloalkane dehalogenase designed to covalently bind to synthetic ligands (HaloTag ligands) (Los et al., 2008, ACS Chem. Biol. 3:373-382). The synthetic ligands comprise a chloroalkane linker attached to a variety of useful molecules. A covalent bond forms between the HaloTag and the chloroalkane linker that is highly specific, occurs rapidly under physiological conditions, and is essentially irreversible.

In some embodiments, a binding agent is joined to a coding tag using a cysteine bioconjugation method. In some embodiments, a binding agent is joined to a coding tag using x-clamp-mediated cysteine bioconjugation (See e.g., Zhang et al., Nat Chem. (2016) 8 (2): 120-128). In some cases, a binding agent is joined to a coding tag using 3-arylpropiolonitriles (APN)-mediated tagging (e.g., Koniev et al., Bioconjug Chem. 2014; 25 (2): 202-206).

In certain embodiments, a coding tag may further comprise a unique molecular identifier for the binding agent to which the coding tag is linked. A UMI for the binding agent may be useful in embodiments utilizing extended coding tags for sequencing readouts, which in combination with the encoder sequence provides information regarding the identity of the binding agent and number of unique binding events for a polypeptide.

### Amino Acid Cleavage

In embodiments relating to methods of analyzing target polypeptides using a degradation based approach, following contacting and binding of a first binding agent to an n NTAA of a peptide of n amino acids, and transferring of the first binding agent's coding tag information to a nucleic acid recording tag associated with the polypeptide, thereby generating a first order extended recording tag, the n NTAA is removed. Removal of the n labeled NTAA by contacting with an enzyme or a chemical reagent converts the n−1 amino acid of the polypeptide to an N-terminal amino acid, which is referred to herein as an n-1 NTAA. A second binding agent is contacted with the polypeptide and binds to the n−1 NTAA, and the second binding agent's coding tag information is transferred to the first order extended recording tag, thereby generating a second order extended recording tag (e.g., for generating a concatenated nth order extended recording tag representing the binding history of the polypeptide, since it contains identifying information about all binding agents that were bound to the polypeptide). Additional functionalization, binding, transfer, and removal can occur as described above up to n amino acids to generate an nth order extended recording tag or n separate extended recording tags, which collectively represent the binding history of the polypeptide.

In certain embodiments relating to analyzing peptides, following binding of a terminal amino acid (N-terminal or C-terminal) by a binding agent and transfer of coding tag information, the terminal amino acid is removed or cleaved from the peptide to expose a new terminal amino acid. In some embodiments, the terminal amino acid is an NTAA. In other embodiments, the terminal amino acid is a CTAA. Cleavage of a terminal amino acid can be accomplished by any number of known techniques, including chemical cleavage and enzymatic cleavage. In some embodiments, an engineered enzyme that catalyzes or reagent that promotes the removal of the PITC-derivatized or other labeled N-terminal amino acid is used. In some embodiments, the terminal amino acid is removed or cleaved using mild Edman-like methods described, for example in US20200348307 A1. In some embodiments, the functionalized NTAA residues of immobilized polypeptide analytes are removed by chemical methods disclosed in U.S patent publication No. 2022/0227889 A1, incorporated herein by reference.

Alternatively, modified cleavase enzymes are used to remove the functionalized NTAA residues of immobilized polypeptide analytes, and the modified cleavase enzymes are disclosed in U.S. Pat. No. 11,427,814, incorporated herein by reference. In some embodiments, the modified cleavase enzyme: (i) is configured to cleave a peptide bond between a terminally labeled amino acid residue and a penultimate terminal amino acid residue of a polypeptide; (ii) is derived from a dipeptidyl aminopeptidase, which removes an unlabeled terminal dipeptide from a polypeptide; (iii) comprises two or more amino acid substitutions in the dipeptidyl aminopeptidase in the residues corresponding to positions N214, W215, R219, N329, D673 and G674 of SEQ ID NO: 19, and comprises an amino acid sequence that exhibits at least 20% sequence identity to SEQ ID NO: 20 (see also U.S. Pat. No. 11,427,814, incorporated herein by reference).

Enzymatic cleavage of a NTAA may be accomplished by an aminopeptidase or other peptidases. Natural aminopeptidases have very limited specificity, and generically cleave N-terminal amino acids in a processive manner, cleaving one amino acid off after another. For the methods described here, aminopeptidases (e.g., metalloenzymatic aminopeptidase) may be engineered to possess specific binding or catalytic activity to the NTAA only when modified with an N-terminal label. For example, an aminopeptidase may be engineered such than it only cleaves an N-terminal amino acid if it is modified by a group such as PTC, modified-PTC, Cbz. DNP, SNP, acetyl, guanidinyl, diheterocyclic methanimine, etc. In this way, the aminopeptidase cleaves only a single amino acid at a time from the N-terminus, and allows control of the degradation cycle. In some embodiments, the modified aminopeptidase is non-selective as to amino acid residue identity while being selective for the N-terminal label. In other embodiments, the modified aminopeptidase is selective for both amino acid residue identity and the N-terminal label. Engineered aminopeptidase mutants that bind to and cleave individual or small groups of labelled (biotinylated) NTAAs have been described (see, e.g., PCT Publication No. WO2010/065322). In certain embodiments, the aminopeptidase may be engineered to be non-specific, such that it does not selectively recognize one particular amino acid over another, but rather just recognizes the labeled N-terminus. In yet another embodiment, cyclic cleavage is attained by using an engineered acylpeptide hydrolase (APH) to cleave an acetylated NTAA. In yet another embodiment, amidination (guanidinylation) of the NTAA is employed to enable mild cleavage of the labeled NTAA using NaOH (Hamada, (2016) Bioorg Med Chem Lett 26 (7): 1690-1695).

In some embodiments, the method further comprises contacting the polypeptide with a proline aminopeptidase under conditions suitable to cleave an N-terminal proline before step (b). In some examples, a proline aminopeptidase (PAP) is an enzyme that is capable of specifically cleaving an N-terminal proline from a polypeptide. PAP enzymes that cleave N-terminal prolines are also referred to as proline iminopeptidases (PIPs). Known monomeric PAPs include family members from B. coagulans, L. delbrueckii, N. gonorrhoeae, F. meningosepticum, S. marcescens, T. acidophilum, L. plantarum (MEROPS S33.001) Nakajima et al., J Bacteriol. (2006) 188 (4): 1599-606; Kitazono et al., Bacteriol (1992) 174 (24): 7919-7925). Known multimeric PAPs including D. hansenii (Bolumar et al., (2003) 86 (1-2): 141-151) and similar homologues from other species (Basten et al., Mol Genet Genomics (2005) 272 (6): 673-679). Either native or engineered variants/mutants of PAPs may be employed.

Analysis after Information Transfer

In some embodiments, the extended recording tag generated from performing the provided methods comprises information transferred from one or more coding tags. In some embodiments, the extended recording tags further comprise identifying information from one or more coding tags. In some embodiments, the extended recording tags are amplified (or a portion thereof) prior to determining at least the sequence of the coding tag(s) in the extended recording tag. In some embodiments, the extended recording tags (or a portion thereof) are released prior to determining at least the sequence of the coding tag(s) in the extended recording tag.

The length of the final extended recording tag generated by the methods described herein is dependent upon multiple factors, including the length of the coding tag(s) (e.g., barcode and spacer), and optionally including any unique molecular identifier, spacer, universal priming site, barcode, or combinations thereof. After transfer of the final tag information to the extended nucleic acid (e.g., from any coding tags), the tag can be capped by addition of a universal reverse priming site via ligation, primer extension or other methods known in the art. In some embodiments, the universal forward priming site in the nucleic acid (e.g., on the recording tag) is compatible with the universal reverse priming site that is appended to the final extended nucleic acid. In some embodiments, a universal reverse priming site is an Illumina P7 primer (5′-CAAGCAGAAGACGGCATACGAGAT-3′-SEQ ID NO:2) or an Illumina P5 primer (5′-AATGATACGGCGACCACCGA-3′-SEQ ID NO:1). The sense or antisense P7 may be appended, depending on strand sense of the nucleic acid to which the identifying information from the coding tag is transferred to. An extended nucleic acid library can be cleaved or amplified directly from the support (e.g., beads) and used in traditional next generation sequencing assays and protocols.

In some embodiments, a primer extension reaction is performed on a library of single stranded extended nucleic acids (e.g., extended on the recording tag) to copy complementary strands thereof. In some embodiments, the peptide sequencing assay (e.g., ProteoCode™ assay), comprises several chemical and enzymatic steps in a cyclical progression.

Extended nucleic acids recording tags can be processed and analyzed using a variety of nucleic acid sequencing methods. In some embodiments, extended recording tags containing the information from one or more coding tags and any other nucleic acid components are processed and analyzed. In some embodiments, the collection of extended recording tags can be concatenated. In some embodiments, the extended recording tag can be amplified prior to determining the sequence.

Examples of sequencing methods include, but are not limited to, chain termination sequencing (Sanger sequencing); next generation sequencing methods, such as sequencing by synthesis, sequencing by ligation, sequencing by hybridization, polony sequencing, ion semiconductor sequencing, and pyrosequencing; and third generation sequencing methods, such as single molecule real time sequencing, nanopore-based sequencing, duplex interrupted sequencing, and direct imaging of DNA using advanced microscopy.

A library of nucleic acids (e.g., extended nucleic acids) may be amplified in a variety of ways. A library of nucleic acids (e.g., recording tags comprising information from one or more coding tags) undergo exponential amplification, e.g., via PCR or emulsion PCR. Emulsion PCR is known to produce more uniform amplification (Hori, Fukano et al., Biochem Biophys Res Commun (2007) 352 (2): 323-328). Alternatively, a library of nucleic acids (e.g., extended nucleic acids) may undergo linear amplification, e.g., via in vitro transcription of template DNA using T7 RNA polymerase. The library of nucleic acids (e.g., extended nucleic acids) can be amplified using primers compatible with the universal forward priming site and universal reverse priming site contained therein. A library of nucleic acids (e.g., the recording tag) can also be amplified using tailed primers to add sequence to either the 5′-end, 3′-end or both ends of the extended nucleic acids. Sequences that can be added to the termini of the extended nucleic acids include library specific index sequences to allow multiplexing of multiple libraries in a single sequencing run, adaptor sequences, read primer sequences, or any other sequences for making the library of extended nucleic acids compatible for a sequencing platform. An example of a library amplification in preparation for next generation sequencing is as follows: a 20 μl PCR reaction volume is set up using an extended nucleic acid library eluted from ˜ 1 mg of beads (˜10 ng), 200 μM dNTP. 1 μM of each forward and reverse amplification primers, 0.5 μl (1 U) of Phusion Hot Start enzyme (New England Biolabs) and subjected to the following cycling conditions: 98° C. for 30 sec followed by 20 cycles of 98° C. for 10 sec, 60° C. for 30 sec, 72° C. for 30 sec, followed by 72° C. for 7 min, then hold at 4° C.

Following sequencing of the nucleic acid libraries (e.g., of extended nucleic acids), the resulting sequences can be collapsed by their UMIs if used and then associated with their corresponding polypeptides and aligned to the totality of the proteome. Resulting sequences can also be collapsed by their compartment tags and associated with their corresponding compartmental proteome, which in a particular embodiment contains only a single or a very limited number of protein molecules. Both protein identification and quantification can easily be derived from this digital peptide information.

### Approaches for Balancing Encoding Signals of Polypeptide Analytes.

Provided herein is a method for analyzing a plurality of different polypeptides immobilized on a support, the method comprising:
(a) contacting the plurality of different polypeptides comprising a first polypeptide and a second polypeptide with a binder, wherein the first polypeptide is associated with a first recording tag and the second polypeptide is associated with a second recording tag, and wherein the binder comprises: (i) a binding moiety capable of binding to the first polypeptide; and (ii) a handle attached to the binding moiety and configured to bind to or react with the first recording tag;
(b) allowing the handle to bind to or react with the first recording tag brought in proximity by binding of the binder to the first polypeptide, thereby modifying the first recording tag to generate a modified first recording tag associated with the first polypeptide;
(c) optionally, fragmenting the plurality of different polypeptides immobilized on the support to generate fragments of different polypeptides immobilized on the support;
(d) contacting the plurality of different polypeptides or the fragments of different polypeptides with a plurality of binding agents, wherein each binding agent comprises: (i) a binding moiety capable of binding to a portion or component of a polypeptide of the plurality of different polypeptides or the fragments thereof; and (ii) a coding tag that comprises identifying information regarding the binding agent;
(c) allowing transfer of identifying information from coding tags of the plurality of binding agents to recording tags associated with the plurality of different polypeptides or the fragments of different polypeptides, thereby generating an extended second recording tag associated with the second polypeptide or fragment thereof upon binding of a binding agent to the second polypeptide or fragment thereof, wherein transfer of identifying information to the modified first recording tag associated with the first polypeptide or fragment thereof is suppressed or blocked; and
(f) analyzing the extended second recording tag to obtain identifying information regarding the binding agent that binds to the second polypeptide or fragment thereof, thereby obtaining information about the second polypeptide or fragment thereof, wherein the analyzing comprises nucleic acid sequencing.

Provided herein is also a method for analyzing a plurality of different polypeptides immobilized on a solid support, comprising the steps of: (a) contacting the plurality of different polypeptides comprising a first polypeptide and a second polypeptide with a binder, wherein the first polypeptide is associated with a first recording tag and the second polypeptide is associated with a second recording tag, and wherein the binder comprises: (i) a binding moiety capable of binding to a first polypeptide of the plurality of different polypeptides; and (ii) a handle attached to the binding moiety and configured to bind or react, when in proximity, to the first recording tag; (b) following binding of the binder to the first polypeptide, providing conditions to allow the handle to bind to or react with the first recording tag, thereby modifying the first recording tag to generate a modified first recording tag associated with the first polypeptide; (c) optionally, fragmenting the plurality of different polypeptides immobilized on the solid support to generate fragments of different polypeptides immobilized on the solid support; (d) contacting the plurality of different polypeptides or the fragments of different polypeptides with a plurality of binding agents, comprising contacting the first polypeptide or a fragment thereof with a first binding agent of the plurality of binding agents, and contacting the second polypeptide or a fragment thereof with a second binding agent of the plurality of binding agents, wherein the first binding agent comprises a first coding tag that comprises identifying information regarding the first binding agent, and the second binding agent comprises a second coding tag that comprises identifying information regarding the second binding agent; (e) providing conditions to allow transfer of identifying information from coding tags of binding agents of the plurality of binding agents to recording tags associated with the plurality of different polypeptides or the fragments of different polypeptides, thereby generating an extended second recording tag associated with the second polypeptide, wherein transfer of identifying information regarding the first binding agent from the first coding tag to the modified first recording tag associated with the first polypeptide is suppressed or blocked; (f) analyzing the extended second recording tag to obtain identifying information regarding the second binding agent, thereby obtaining information about the second polypeptide, wherein the analyzing comprises using nucleic acid sequencing.

Provided herein is also a method for analyzing molecules of a polypeptide immobilized on a support, the method comprising:
(a) contacting the molecules of the polypeptide with a first binding agent and a second binding agent,
wherein each molecule of the polypeptide is associated with a recording tag immobilized on a support,
wherein the first binding agent comprises (i) a first binding moiety capable of binding to the polypeptide; and (ii) a first coding tag attached to the first binding moiety and comprising identifying information regarding the first binding agent, and
wherein the second binding agent comprises (i) a second binding moiety capable of binding to the polypeptide; and, optionally, (ii) a handle attached to the second binding moiety and configured to bind to or react with the recording tag;
(b) allowing transfer of the identifying information regarding the first binding agent from the first coding tag to the recording tag by primer extension and/or ligation to generate an extended recording tag, and optionally, allowing the handle to bind to or react with the first recording tag to generate a modified first recording tag;
(c) contacting the molecules of the polypeptide with a third binding agent comprising (i) a third binding moiety capable of binding to the polypeptide; and (ii) a third coding tag attached to the third binding moiety and comprising identifying information regarding the third binding agent;
(d) allowing transfer of the identifying information regarding the third binding agent from the third coding tag to the extended recording tag by primer extension and/or ligation to generate a further extended recording tag, wherein transfer of the identifying information regarding the third binding agent from the third coding tag to the recording tag or to the modified first recording tag is suppressed or blocked; and
(e) analyzing the further extended recording tag to obtain identifying information regarding the first binding agent and/or the third binding agent, thereby obtaining information about the polypeptide, wherein the analyzing comprises nucleic acid sequencing.

Provided herein is also a method for analyzing a plurality of different polypeptides immobilized on a solid support, comprising the steps of:
(a) contacting a first polypeptide of the plurality of different polypeptides with a set of binding agents, wherein the first polypeptide is associated with a first recording tag immobilized on a solid support, and wherein a first binding agent of the set of binding agents comprises: (i) a binding moiety capable of binding to the first polypeptide; and (ii) a first coding tag attached to the binding moiety and comprising identifying information regarding the first binding agent; and a second binding agent of the at least two binding agents comprises (i) the binding moiety essentially identical to the binding moiety of the first binding agent; and, optionally, (ii) a handle attached to the binding moiety of the second binding agent and configured to bind, when in proximity, to the first recording tag associated with the first polypeptide;
(b) following binding of either the first or second binding agent to the first polypeptide, providing conditions to allow transfer of the identifying information regarding the first binding agent from the first coding tag to the first recording tag by primer extension or ligation to generate an extended first recording tag, or to allow the handle to bind to the first recording tag generating a modified first recording tag;
(c) contacting the first polypeptide with a third binding agent capable of binding to the first polypeptide, wherein the third binding agent comprises a binding moiety capable of binding to the first polypeptide and a third coding tag that comprises identifying information regarding the third binding agent;
(d) providing conditions to allow transfer of the identifying information regarding the third binding agent from the third coding tag to the extended first recording tag by primer extension or ligation to generate a further extended first recording tag, wherein transfer of the identifying information regarding the third binding agent from the third coding tag to the first recording tag or to the modified first recording tag is suppressed or blocked;
(c) analyzing the further extended recording tag to obtain identifying information regarding the first binding agent and/or the third binding agent, thereby obtaining information about the first polypeptide, wherein the analyzing comprises using nucleic acid sequencing.

Various embodiments apply equally to the aspects provided herein but will for the sake of brevity be recited only once. Thus, various of the following embodiments apply equally to aspects recited below.

Exemplary embodiments of the disclosed methods are illustrated in FIGS. 3A-3B, FIG. 4-FIG. 6, and FIGS. 7A-7B.

FIGS. 3A-3B show exemplary “negative” capping of abundant protein B before performing the NGPS peptide sequencing assay. Abundant protein B was targeted with a binding agent (such as a specific antibody or aptamer) comprising a coding tag that upon binding to protein B modifies the recording tag associated with protein B, adding an extension spacer B. Next, the plurality of proteins was digested and peptides that remain immobilized on the solid support are subjected to the NGPS peptide sequencing assay. Only peptides associated with the unmodified recording tags are encoded (such as peptide formed from protein A), but encoding of peptide formed from protein B is blocked, since the extension spacer B is not complementary to the spacer on the coding tag of the binding agent that binds peptides during the NGPS peptide assay (e.g., the binding agent that binds to a specific functionalized NTAA residue of peptides).

In some embodiments, to perform “negative” capping, an antibody to an abundant protein, whose signal needs to be blocked or suppressed, comprises a “terminating” coding tag (cTag) which drives primer extension of the recording tag (rTag) associated with the abundant protein, creating a non-functional spacer element.

FIG. 4 and FIG. 5 show exemplary embodiments of “negative” capping of abundant protein B prior to NGPS peptide sequencing assay. Abundant protein B was targeted with a binding agent comprising a chemical moiety or an enzyme (e.g., a nuclease) that upon binding to protein B modifies the recording tag associated with protein B. Only peptides associated with the unmodified recording tags are encoded in the following NGPS peptide assay (such as peptide formed from protein A), but encoding of peptide formed from protein B is blocked, since the associated recording tag is rendered nonfunctional or destroyed. In some embodiments, the modification of the recording tag and the consequent blocking of encoding is irreversible. In some embodiments, the modification of the recording tag is reversible and the encoding involving the recording tag may be resumed, for instance, by reversing a chemical or enzymatic reaction and/or regenerating a functional recording tag.

FIG. 6 shows an exemplary embodiment of “positive” and “negative” capping of proteins A and B prior to NGPS peptide sequencing assay. Prior to NGPS assay, protein A and protein B are contacted with binding agents specific for protein A and for protein B, and conjugated to coding tags comprising either SP_B′ or SP_C′ spacer sequences. Following binding of the binding agents to protein A and protein B, the recording tags associated with protein A and protein B are extended to contain either SP_B (compatible) or SP_C spacer (incompatible) sequences. Next, the plurality of proteins is subjected to protease digestion followed by the NGPS peptide assay. Only peptides associated with recording tags having compatible spacers are encoded in the next round of binding/encoding (e.g., peptide from Protein A), whereas peptides associated with recording tags having incompatible spacers cannot be encoded (e.g., peptide from Protein B). In another embodiment (related but not shown in FIG. 6), binding agent specific for protein B may not comprise a coding tag or a moiety. Following binding of the binding agents to protein A and protein B, the recording tag associated with protein A is extended to contain SP_B (compatible), whereas the recording tag associated with protein B remain unmodified (since there is no tag transfer). Again, the plurality of proteins is subjected to protease digestion followed by the NGPS peptide assay. Only peptides associated with recording tags having compatible spacers are encoded in the next round of binding/encoding (e.g., peptide from Protein A), whereas peptides associated with unmodified recording tags have incompatible spacers for the next round of binding/encoding and cannot be extended/encoded (e.g., peptide from Protein B).

FIGS. 7A-7B show an exemplary competitive attenuation of encoding during NGPS assay for an abundant protein A. Prior to NGPS assay, protein A is contacted with a mixture of binding agents specific for Protein A and conjugated with coding tag comprising either spacer SP_B (compatible, 1% of binding agent molecules) or SP_X (in compatible, 99% of binding agent molecules). Following binding of the binding agents to Protein A, the recording tags associated with Protein A are extended (modified) to contain either SP_B′ or SP_X′ spacer sequences. Next, the plurality of proteins is digested and subjected to the NGPS peptide sequencing assay. Only protein A associated with the recording tag having the compatible SP_B′ spacer sequence is encoded in the next round of binding/encoding, whereas encoding of the recording tag having the incompatible SP_X′ spacer sequence is blocked. In another embodiment (related but not shown in FIGS. 7A-7B), protein A is contacted with a mixture of binding agents specific for Protein A, wherein one agent is conjugated with a coding tag comprising spacer SP_B (compatible, e.g., 1% of binding agent molecules), while another agent (having essentially the same binding moiety specific for Protein A) does not have a coding tag. Following binding of the binding agents to protein A, the recording tag associated with a portion of protein A molecules is extended to contain SP_B (compatible) spacer, whereas the recording tag associated with other protein A molecules remain unmodified (since there is no tag transfer). Accordingly, only protein A molecules that are associated with recording tags having the compatible (and thus functional) spacer can be further extended in the next round of binding/encoding; thus, the encoding signal from protein A can be effectively reduced by controlling ratio of binding agents comprising compatible vs incompatible spacers (or compatible spacer vs no spacer).

FIGS. 9A-9C and FIGS. 10A-10C show exemplary immobilization techniques for native or derivatized proteins on NGPA ProteoCode™ beads. Immobilized proteins can then be analyzed by NGPA ProteoCode™ assay shown in FIG. 1A or FIG. 1C. Alternatively, immobilized proteins can also be analyzed by NGPS peptide sequencing assay.

In some embodiments, any one or a combination of the approaches described in FIGS. 11A-11C can be used to generate a mixture of modifying binders (e.g., as shown in FIG. 11A or FIG. 11B) and non-modifying binders (e.g., as shown in FIG. 11C), and control ratio of the binders in the mixture to obtain a desirable attenuation or suppression of encoding (e.g., of an abundant polypeptide in a sample).

The methods disclosed herein can be used for analysis, including detection, quantitation and/or sequencing, of a plurality of polypeptides simultaneously (multiplexing). In some embodiments, multiple polypeptides from a biological sample are analyzed by NGPS assay, and the disclosed methods are used to suppress or block encoding signals produced by relatively abundant polypeptides (for example, polypeptides having high concentrations in the sample, such as polypeptides having concentrations higher than 95^{th} percentile of polypeptide concentrations) in order to process encoding signals produced by polypeptides having medium or low concentrations in the sample. In some embodiments, suppression of the encoding signal for a particular polypeptide can be complete (100% suppression) or partial (e.g., at least 20%, at least 30%, at least 40%, at least 50%, at least 60%, at least 70%, at least 80%, at least 90%, or at least 95% suppression). For example, in some embodiments, transfer of identifying information regarding the first binding agent from the first coding tag to the modified first recording tag associated with the first polypeptide is suppressed by at least 20%, at least 30%, at least 40%, at least 50%, at least 60%, at least 70%, at least 80%, at least 90%, at least 95% or at least 99%. In some embodiments, transfer of identifying information regarding the first binding agent from the first coding tag to the modified first recording tag associated with the first polypeptide is blocked (e.g., not possible, under the same conditions that permit information transfer to an unmodified recording tag), for example, when the modified first recording tag is unfunctional (e.g., completely or partially destroyed by the handle or modified by the handle in a way that prevents further information transfer by ligation and/or primer extension).

In some embodiments, the disclosed methods for analyzing a plurality of different polypeptides comprise methods for analyzing at least 50, at least 100, at least 500, at least 1000, at least 10000, at least 100,000, or at least 1,000,000 different polypeptides.

In preferred embodiments of the present disclosure, contacting the polypeptide with the first, second or higher order binding agent results in binding of the binding agent to a component of the polypeptide. The cognate binding agent can bind specifically to either unmodified component of the polypeptide or modified component of the polypeptide. For example, a component of the polypeptide is modified with a modifier agent, and the cognate binding agent has binding affinity to the modified component of the polypeptide.

In preferred embodiments, the nucleic acid recording tag associated with the polypeptide is an element of the disclosed assay and is not a component of the polypeptide. Thus, binding agents of the present methods do not bind to the nucleic acid recording tag.

In some embodiments of the method, step (a) comprises providing a plurality of polypeptides and associated nucleic acid recording tags joined to the solid support; and each of steps (b) and (d) comprises contacting the plurality of polypeptides with a first or higher order plurality of binding agents capable of binding to the polypeptides, wherein the first or higher order plurality of binding agents comprise a first or higher order nucleic acid coding tag(s) with identifying information regarding the first or higher order plurality of binding agents.

In some embodiments, providing the polypeptide and an associated recording tag joined to a solid support comprises the following steps: attaching the polypeptide to the recording tag to generate a nucleic acid-polypeptide conjugate; bringing the nucleic acid-polypeptide conjugate into proximity with a solid support by hybridizing the recording tag in the nucleic acid-polypeptide conjugate to a capture nucleic acid attached to the solid support; and covalently coupling the nucleic acid-polypeptide conjugate to the solid support.

In some embodiments, providing the polypeptide and an associated recording tag joined to a solid support further comprises attaching the polypeptide analyte to the nucleic acid recording tag optionally joined to the solid support.

In some embodiments of the method, the plurality of polypeptides is spaced apart on the solid support at an average distance >50 nm.

In some preferred embodiments, the method further comprises modifying the N-terminal amino acid (NTAA) of the polypeptide with a chemical moiety to produce a modified NTAA. In some embodiments of the method, the second or higher order binding agent is capable of binding to the modified NTAA. In some embodiments, the method further comprises removing the modified NTAA to expose a new NTAA of the polypeptide. In some embodiments of the method, the extended nucleic acid recording tag obtained after extension reactions of step (e) is amplified prior to analysis.

In some embodiments of the method, the DNA polymerase is an engineered DNA polymerase having a reduced template independent nucleotide addition ability during the primer extension reaction. Some examples of such engineered DNA polymerases are disclosed in U.S. Pat. No. 7,501,237 B2 which is incorporated herein by reference.

In some embodiments, the primer extension reaction performed during information transfer step is performed under conditions to reduce or prevent template independent nucleotide addition by the polymerase during the primer extension reaction. In some embodiments, the conditions of the primer extension reaction performed during information transfer step decrease yield of template independent nucleotide addition to 10% or less. In some embodiments, the yield of template independent nucleotide addition during the primer extension reaction is below 10%. In some embodiments, the yield of template independent nucleotide addition during the primer extension reaction is below 5%. In some embodiments, the yield of template independent nucleotide addition during the primer extension reaction is below 1%.

In some embodiments, providing the polypeptide and an associated recording tag joined to a solid support comprises the following steps: attaching the polypeptide to the recording tag to generate a nucleic acid-polypeptide conjugate; bringing the nucleic acid-polypeptide conjugate into proximity with a solid support by hybridizing the recording tag in the nucleic acid-polypeptide conjugate to a capture nucleic acid attached to the solid support; and covalently coupling the nucleic acid-polypeptide conjugate to the solid support.

In some embodiments, providing the polypeptide and an associated recording tag joined to a solid support further comprises attaching the polypeptide analyte to the nucleic acid recording tag optionally joined to the solid support.

In some embodiments, the nucleic acid recording tag is associated directly or indirectly to the polypeptide analyte via a non-nucleotide chemical moiety.

In some embodiments of the disclosed methods, nucleic acid tags (such as recording tags or coding tags) are used that comprise a barcode (e.g., a sample barcode, a fraction barcode, spatial barcode, a compartment tag, etc.) and/or an optional UMI. In some embodiments, error-correcting or error-tolerant barcodes can be used to decrease potential errors during analyzing and decoding of barcode sequences. Nucleic acid barcodes can be designed to be tolerant to error-prone NGS sequencers, such as nanopore-based sequencers where the current base call error rate is around 5-10%. A number of error correcting code systems have been described in the art and can be used herein. These include Hamming codes, Lee distance codes, asymmetric Lee distance codes, Reed-Solomon codes, Levenshtein codes, and others. Error-tolerant barcodes can be generated based on Hamming and Levenshtein codes using R Bioconductor package, “DNAbarcodes”, which are capable of correcting insertion, deletion, and substitution errors, depending on the design parameters chosen (see, e.g., US20190145982 A1 and Buschmann and Bystrykh, 2013, Levenshtein error-correcting barcodes for multiplexed DNA sequencing. BMC Bioinformatics 14, 272 (2013), incorporated herein by reference).

In some embodiments, providing conditions to allow transfer of identifying information from a coding tag of the binding agent to a recording tag associated with the polypeptide comprises addition of an enzyme (such as a DNA polymerase or a DNA ligase) to the immobilized polypeptide, as well as an appropriate buffer for this enzyme (such as a buffer for DNA polymerase or DNA ligase). Standard buffers that provide functionality of DNA polymerase or DNA ligase are known in the art.

In preferred embodiments, to provide encoding reaction specificity, transfer of identifying information regarding a binding agent from a coding tag of the binding agent to a recording tag associated with an immobilized polypeptide occurs only following (or after) binding of the binding agent to the immobilized polypeptide. The binding agent binds specifically to a component of the immobilized polypeptide (in various embodiments, binds to a single NTAA residue, to a modified amino acid residue, such as residue modified post-translationally, to an epitope, or to more than one epitopes simultaneously); and binding of the binding agent to the immobilized polypeptide does not depend on the presence of the recording tag associated with the immobilized polypeptide.

In some embodiments, before providing the polypeptide analyte and the associated nucleic acid recording tag joined to the solid support, the provided methods further comprise attaching the polypeptide analyte to the nucleic acid recording tag optionally joined to the solid support. Various alternatives can be used during the attachment step. For example, the polypeptide analyte can first be attached to the nucleic acid recording tag forming a conjugate, and then the conjugate is attached to the solid support. Alternatively, the nucleic acid recording tag can be attached (immobilized) to the solid support, and then the polypeptide analyte is attached to the immobilized nucleic acid recording tag.

In some embodiments, when a plurality of the nucleic acid-polypeptide conjugates is coupled on the solid support, any adjacently coupled nucleic acid-polypeptide conjugates are spaced apart from each other at an average distance of about 50 nm or greater.

In some embodiments, the polypeptide is attached to the 3′ end of the recording tag. In other embodiments, the polypeptide is attached to the 5′ end of the recording tag. In yet other embodiments, the polypeptide is attached to an internal position of the recording tag.

In some embodiments, a barcode is attached to the nucleic acid-polypeptide conjugate, wherein the barcode comprises a compartment barcode, a partition barcode, a sample barcode, a fraction barcode, or any combination thereof.

In some embodiments, the recording tag is covalently attached to the polypeptide to generate the nucleic acid-polypeptide conjugate. In some embodiments, the recording tag and/or capture nucleic acid further comprises a universal priming site, wherein the universal priming site comprises a priming site for amplification, sequencing, or both.

In some embodiments, the capture nucleic acid (e.g., capture DNA) is used to immobilize polypeptide analytes (or polypeptide-nucleic acid conjugates) on a solid support. In some embodiments, the capture nucleic acid is derivatized or comprises a moiety (e.g., a reactive coupling moiety) to allow binding to a solid support. In some embodiments, the capture nucleic acid comprises a moiety (e.g., a reactive coupling moiety) to allow binding to the recording tag. In some other embodiments, the recording tag is derivatized or comprises a moiety (e.g., a reactive coupling moiety) to allow binding to a solid support. Methods of derivatizing a nucleic acid for binding to a solid support and reagents for accomplishing the same are known in the art. For this purpose, any reaction which is preferably rapid and substantially irreversible can be used to attach nucleic acids to the solid support. The capture nucleic acid may be bound to a solid support through covalent or non-covalent bonds. In a preferred embodiment, the capture nucleic acid is covalently bound to biotin to form a biotinylated conjugate. The biotinylated conjugate is then bound to a solid support, for example, by binding to a solid, insoluble support derivatized with avidin or streptavidin. The capture nucleic acid can be derivatized for binding to a solid support by incorporating modified nucleic acids in the loop region. In other embodiments, the capture moiety is derivatized in a region other than the loop region.

Exemplary bioorthogonal reactions that can be used for binding of polypeptides and/or associated recording tags to a solid support or for generating nucleic acid-polypeptide conjugates include the copper catalyzed reaction of an azide and alkyne to form a triazole (Huisgen 1, 3-dipolar cycloaddition), strain-promoted azide alkyne cycloaddition (SPAAC), reaction of a diene and dienophile (Diels-Alder), strain-promoted alkyne-nitrone cycloaddition, reaction of a strained alkene with an azide, tetrazine or tetrazole, alkene and azide [3+2] cycloaddition, alkene and tetrazine inverse electron demand Diels-Alder (IEDDA) reaction (e.g., m-tetrazine (mTet) or phenyl tetrazine (pTet) and trans-cyclooctene (TCO); or pTet and an alkene), alkene and tetrazole photoreaction, Staudinger ligation of azides and phosphines, and various displacement reactions, such as displacement of a leaving group by nucleophilic attack on an electrophilic atom (Horisawa 2014, Knall, Hollauf et al. 2014). Exemplary displacement reactions include reaction of an amine with: an activated ester; an N-hydroxysuccinimide ester; an isocyanate; an isothioscyanate, an aldehyde, an epoxide, or the like. In some embodiments, iEDDA click chemistry is used for immobilizing polypeptides to a solid support or for generating nucleic acid-polypeptide conjugates since it is rapid and delivers high yields at low input concentrations. In another embodiment, m-tetrazine rather than tetrazine is used in an iEDDA click chemistry reaction, as m-tetrazine has improved bond stability. In another embodiment, phenyl tetrazine (pTet) is used in an iEDDA click chemistry reaction.

In some embodiments, a plurality of capture nucleic acids is coupled to the solid support. In some cases, the sequence region that is complementary to the recording tag on the capture nucleic acids is the same among the plurality of capture nucleic acids. In some cases, the recording tag attached to various polypeptides comprises the same complementary sequence to the capture nucleic acid.

In some embodiments, the surface of the solid support is passivated (blocked). A “passivated” surface refers to a surface that has been treated with outer layer of material. Methods of passivating surfaces include standard methods from the fluorescent single molecule analysis literature, including passivating surfaces with polymer like polyethylene glycol (PEG) (Pan et al., 2015, Phys. Biol. 12:045006), polysiloxane (e.g., Pluronic F-127), star polymers (e.g., star PEG) (Groll et al., 2010, Methods Enzymol. 472:1-18), hydrophobic dichlorodimethylsilane (DDS)+self-assembled Tween-20 (Hua et al., 2014, Nat. Methods 11:1233-1236), diamond-like carbon (DLC), DLC+PEG (Stavis et al., 2011, Proc. Natl. Acad. Sci. USA 108:983-988), and zwitterionic moieties (e.g., U.S. Patent Application Publication US 2006/0183863). In addition to covalent surface modifications, a number of passivating agents can be employed as well including surfactants like Tween-20, polysiloxane in solution (Pluronic series), poly vinyl alcohol (PVA), and proteins like BSA and casein. Alternatively, density of polypeptides (e.g., proteins, polypeptide, or peptides) can be titrated on the surface or within the volume of a solid support by spiking a competitor or “dummy” reactive molecule when immobilizing the proteins, polypeptides or peptides to the solid support. In some embodiments, PEGs of various molecular weights can also be used for passivation from molecular weights of about 300 Da to 50 kDa or more.

In certain embodiments where multiple nucleic acid-polypeptide conjugates are immobilized on the same solid support, the nucleic acid-polypeptide conjugates can be spaced appropriately to accommodate methods of identification to be used. For example, it may be advantageous to space the nucleic acid-polypeptide conjugates that optimally to allow a nucleic acid-based method for identifying the polypeptides to be performed. In some embodiments, the method for analyzing polypeptides involve transferring information of the nucleic acid coding tag attached to a binding agent to the nucleic acid recording tag to generate an extended nucleic acid recording tag, and information transfer from the coding tag may reach a neighboring recording tag.

To control polypeptide spacing or nucleic acid-polypeptide conjugate spacing on the solid support, the density of functional coupling groups (e.g., TCO) may be titrated on the support surface. In some embodiments, adjacently coupled polypeptides or nucleic acid-polypeptide conjugates are spaced apart from each other on the surface or within the volume (e.g., porous supports) of a solid support at an average distance of about 50 nm to about 500 nm, or about 50 nm to about 400 nm, or about 50 nm to about 300 nm, or about 50 nm to about 200 nm, or about 50 nm to about 100 nm. In some embodiments, adjacently coupled polypeptides or nucleic acid-polypeptide conjugates are spaced apart from each other on the surface of a solid support with an average distance of at least 50 nm or at least 100 nm. In some embodiments, adjacently coupled polypeptides or nucleic acid-polypeptide conjugates are spaced apart from each other on the surface or within the volume of a solid support such that, empirically, the relative frequency of inter- to intra-molecular events (e.g. transferring information from the complementary coding tag to a neighboring recording tag vs. the cognate recording tag is <1:10; <1:100; <1:1,000; or <1:10,000. In some embodiments, the plurality of nucleic acid-polypeptide conjugate is coupled on the solid support such that any adjacently coupled nucleic acid-polypeptide conjugates are spaced apart from each other at an average distance which ranges from about 50 to 500 nm, from about 50 to 1000 nm, from about 50 to 1500 nm, from about 50 to 2000 nm.

In some embodiments, the spacing of the polypeptide on the solid support is achieved by controlling the concentration and/or number of capture nucleic acids on the solid support. In some embodiments, any adjacently coupled capture nucleic acids are spaced apart from each other on the surface or within the volume (e.g., porous supports) of a solid support at a distance of about 50 nm, about 100 nm, or about 200 nm. In some embodiments, any adjacently coupled capture nucleic acids are spaced apart from each other on the surface of a solid support with an average distance of at least 50 nm. In some embodiments, any adjacently coupled capture nucleic acids are spaced apart from each other on the surface or within the volume of a solid support such that, empirically, the relative frequency of inter- to intra-molecular events (e.g. transfer of information) is <1:10; <1:100; <1:1,000; or <1:10,000.

A suitable spacing frequency can be determined empirically using a functional assay and can be accomplished by dilution and/or by spiking a “dummy” spacer molecule that competes for attachments sites on the support surface. For example, PEG-5000 (MW˜5000) is used to block the interstitial space between peptides on the support surface (e.g., bead surface). In addition, the peptide is coupled to a functional moiety that is also attached to a PEG-5000 molecule. In some embodiments, the functional moiety is an aldehyde, an azide/alkyne, or a malemide/thiol, or an epoxide/nucleophile, or an inverse electron demand Diels-Alder (iEDDA) group, or a moiety for a Staudinger reaction. In some embodiments, the functional moiety is an aldehyde group. In a preferred embodiment, this is accomplished by coupling a mixture of NHS-PEG-5000-TCO+NHS-PEG-5000-Methyl to amine-derivatized beads. The stoichiometric ratio between the two PEGs (TCO vs. methyl) is titrated to generate an appropriate density of functional coupling moieties (TCO groups) on the support surface; the methyl-PEG is inert to coupling. The effective spacing between TCO groups can be calculated by measuring the density of TCO groups on the surface. In certain embodiments, the mean spacing between coupling moieties (e.g., TCO) on the solid support is at least 50 nm, at least 100 nm, at least 250 nm, or at least 500 nm. After PEG5000-TCO/methyl derivatization of the beads, the excess NH₂ groups on the surface are quenched with a reactive anhydride (e.g. acetic or succinic anhydride).

In some embodiments, the spacing is accomplished by titrating the ratio of available attachment molecules on the support surface. In some examples, the support surface (e.g., bead surface) is functionalized with a carboxyl group (COOH) which is treated with an activating agent (e.g., activating agent is EDC and Sulfo-NHS). In some examples, the support surface (e.g., bead surface) comprises NHS moieties. In some embodiments, a mixture of mPEGₙ-NH₂ and NH₂-PEGₙ-mTet is added to the activated beads (wherein n is any number, e.g., any number from n=1 to n=100 or more). In one example, the ratio between the mPEG₃-NH₂ (not available for coupling) and NH₂-PEG₄-mTet (available for coupling) is titrated to generate an appropriate density of functional moieties available to attach the polypeptide on the support surface. In certain embodiments, the mean spacing between coupling moieties (e.g., NH₂-PEG₄-mTet) on the solid support is at least 50 nm, at least 100 nm, at least 250 nm, or at least 500 nm. In some specific embodiments, the ratio of NH₂-PEGₙ-mTet to mPEGₙ-NH₂ is about or greater than 1:1000, about or greater than 1:10,000, about or greater than 1:100,000, or about or greater than 1:1,000,000. In some further embodiments, the capture nucleic acid attaches to the NH₂-PEGₙ-mTet.

In some embodiments, the DNA polymerase used for primer extension reaction is an engineered DNA polymerase having a reduced template independent nucleotide addition ability during the primer extension reaction.

In some preferred embodiments, the binding agent used to contact the plurality of different polypeptides comprises (i) a binding moiety capable of binding to a first polypeptide from the plurality of different polypeptides; and (ii) a handle attached to the binding moiety and configured to bind, when in close proximity, to a first recording tag associated with the first polypeptide. Following binding of the binding agent to the first polypeptide, the handle is configured to bind to the first recording tag and generate a modified (non-functional) first recording tag. Multiple approaches can be utilized to generate the modified (non-functional) first recording tag, and thus suppress or block transfer of identifying information regarding the first binding agent from a first coding tag of the first binding agent to the first recording tag associated with the first polypeptide.

In some embodiments, the handle comprises a polynucleotide. In some embodiments, the handle binds to the first recording tag by nucleic acid hybridization or via split ligation. Binding of the nucleic acid handle to the first recording tag generates a modified first recording tag comprising an additional spacer sequence at 3′ end via primer extension or ligation. Therefore, on the next steps of the method, following binding of the plurality of binding agents to the first polypeptide that is associated with the modified first recording tag, transfer of identifying information regarding the first binding agent to the modified first recording tag associated with the first polypeptide is suppressed or blocked. In other embodiments, the handle does not comprise a polynucleotide, and transfer of identifying information regarding the first binding agent to the modified first recording tag is suppressed or blocked by a different approach.

In some embodiments, upon binding to the first recording tag, the handle modifies the first recording tag to produce an unfunctional first recording tag associated with the first polypeptide.

In some embodiments, the handle comprises a protein enzyme. For example, the handle may comprise a nuclease, a restriction enzyme or a nucleic acid-modifying enzyme that acts locally on the first recording tag the due to covalent attachment to the binding moiety of the binding agent, and only following binding of the binding agent to the first polypeptide. The first recording tag becomes unfunctional (modified) after exposure to the handle.

In some embodiments, the handle is or comprises a small chemical moiety capable of reacting with the first recording tag to produce an unfunctional first recording tag associated with the first polypeptide.

In some embodiments, the handle is or comprises a small chemical moiety capable of reacting with the first recording tag to produce an unfunctional first recording tag associated with the first polypeptide.

In some embodiments, following binding of the handle to the first recording tag, the handle remains attached to the first recording tag to form an unfunctional first recording tag associated with the first polypeptide.

In some embodiments, the transfer of identifying information regarding the second binding agent from the second coding tag to the second recording tag comprises: (o) generating a double stranded second recording tag by (i) joining at least one end of the second recording tag to an end of the second coding tag by a nucleic acid joining reagent, and (ii) optionally, extending the double stranded second recording tag using the second coding tag as a template by a polymerase; and (p) cleaving the double stranded second recording tag with a double strand nucleic acid cleaving reagent to generate the extended second recording tag.

In some embodiments, the cleavage in step (p) releases the second binding agent from the second polypeptide.

In some embodiments, the double-stranded nucleic acid is cleaved by a nuclease, optionally wherein the nuclease is a restriction enzyme.

In some embodiments, the modified first recording tag does not contain a cleavage site for the restriction enzyme, so that information transfer to this modified first recording tag is blocked. In some embodiments, cleavage site for the restriction enzyme is removed by the handle configured to bind to or react with the first recording tag.

In some embodiments, the 5′ end of the second recording tag is joined to the 3′ end of the second coding tag by a ligase, with or without gap filling prior to ligation by the ligase.

In some embodiments, the second recording tag comprises a hairpin, and the 3′ end of the second recording tag is extended using the second coding tag as the template to generate the double-stranded nucleic acid.

In some embodiments, the cleavage facilitates release of the second binding agent from the second polypeptide or fragment thereof.

In some embodiments, the contacting and transferring steps (e.g., steps (d) and (c)) are repeated sequentially one or more times in a cyclic manner. In some of these embodiments, the disclosed methods further comprise removing a portion of the second polypeptide prior to repeating the contacting step (step (d)). In some of these embodiments, the disclosed methods further comprise removing the N-terminal amino acid (NTAA) of the second polypeptide to expose a new NTAA of the second polypeptide prior to repeating the contacting step (step (d)). In some of these embodiments, a 3′ overhang of the second extended recording tag is generated by the double strand nucleic acid cleaving reagent in the cleaving step (p), and the 3′ overhang is available to hybridize with another coding tag when the contacting step (step (d)) is repeated.

Provided herein is also a method for analyzing a plurality of different polypeptides from a sample, comprising the steps of: (a) providing a first polypeptide from the plurality of different polypeptides and an associated first recording tag immobilized on a solid support; (b) contacting the first polypeptide with at least two binding agents, wherein a first binding agent of the at least two binding agents comprises: (i) a binding moiety capable of binding to the first polypeptide immobilized on the solid support; and (ii) a first coding tag attached to the binding moiety and comprising identifying information regarding the first binding agent; and a second binding agent of the at least two binding agents comprises (i) the binding moiety essentially identical to the binding moiety of the first binding agent; and, optionally, (ii) a handle attached to the binding moiety of the second binding agent and configured to bind, when in close proximity, to the first recording tag associated with the first polypeptide; (c) following binding of either the first or second binding agent to the first polypeptide, providing conditions to allow transfer of the identifying information regarding the first binding agent from the first coding tag to the first recording tag by primer extension or ligation to generate an extended first recording tag, or to allow the handle to bind to the first recording tag generating a modified first recording tag; (d) contacting the first polypeptide with a third binding agent capable of binding to the first polypeptide, wherein the third binding agent comprises a binding moiety capable of binding to the first polypeptide analyte and a third coding tag that comprises identifying information regarding the third binding agent; (c) following binding of the third binding agent to the first polypeptide, providing conditions to allow transfer of the identifying information regarding the third binding agent from the third coding tag to the extended first recording tag by primer extension or ligation to generate a further extended first recording tag, wherein transfer of the identifying information regarding the third binding agent from the third coding tag to the first recording tag or to the modified first recording tag is suppressed or blocked; (f) analyzing the further extended recording tag, wherein analyzing comprises a sequencing method, and obtaining the identifying information regarding the first binding agent, and/or the third binding agent to provide information regarding the first polypeptide; and (g) performing steps (a)-(f) for a second polypeptide from the plurality of different polypeptides, wherein (i) corresponding first, second and third binding agents for the second polypeptide comprise binding moieties capable of binding to the second polypeptide; and (ii) a ratio between quantities of the first binding agent and the second binding agent at step (b) is different for the first and the second polypeptides.

In some embodiments, the ratio between quantities of the first binding agent and the second binding agent at step (b) is calculated before performing the step (b) based on abundance of the first and the second polypeptides in the sample.

In some embodiments, the binding moiety of the first binding agent is identical to the binding moiety of the second binding agent.

In some embodiments, the second binding agent comprises or consists of the binding moiety essentially identical to the binding moiety of the first binding agent and does not comprise the handle.

In some embodiments, the third binding agent comprises a binding moiety capable of binding to the first or second peptide analyte, and the binding moiety of the third binding agent is capable of binding to one or more N-terminal or C-terminal amino acid residues of the peptide analyte, or capable of binding to the one or more N-terminal or C-terminal amino acid residues modified by a functionalizing reagent.

In some embodiments, the second binding agent comprises or consists of the binding moiety essentially identical to the binding moiety of the first binding agent. If the binding moiety of the first binding agent comprises a protein molecule, then the binding moiety of the second binding agent comprises the same or highly homologous protein molecule, which is configured to bind to the same component of the polypeptide analyte. In some embodiments, the sequences of the protein molecules present in the binding moieties differ by no more than 30%, 20%, 10%, 5% or 1%. In preferred embodiments, both protein molecules present in the binding moieties have essentially the same binding affinity to the immobilized polypeptide (or to a component of the polypeptide), and share the same recognition region(s) in the immobilized polypeptide.

In some embodiments, molecules of the first polypeptide are more abundant than molecules of the second polypeptide, and the ratio between amounts of the first binding agent and the second binding agent for the first polypeptide is smaller than the ratio for the second polypeptide. In some embodiments, the ratio for the first polypeptide and/or the ratio for the second polypeptide is selected or determined before contacting the molecules of the first and/or second polypeptides with the corresponding first and second binding agents. In some embodiments, the method further comprises estimating relative abundance of the first and the second polypeptides in a biological sample, wherein the relative abundance correlates with the relative abundance of the first and the second polypeptides immobilized on supports.

In some embodiments, at step (b), the first polypeptide and second polypeptide are contacted at the same time with the same set of binding agents that comprises binding agents capable of binding to the first polypeptide and to the second polypeptide.

In some embodiments, the first polypeptide and second polypeptide are immobilized on different solid supports, which are mixed and treated together. In some other embodiments, the first polypeptide and second polypeptide are immobilized on the same solid support.

In preferred embodiments, the modified recording tag is an unfunctional recording tag associated with the polypeptide, which implies that the unfunctional recording tag does not support further transfer of identifying information regarding the next cognate binding agent bound to the polypeptide from the coding tag of the binding agent. In several different embodiments, the unfunctional (modified) recording tag may be degraded by a nuclease, modified by a chemical entity, or modified to contain a terminal spacer that is not compatible with nucleic acid hybridization-based interaction with the coding tag during the next encoding cycle.

In some embodiments, transfer of identifying information regarding the first binding agent from the first coding tag of the first binding agent to the modified first recording tag associated with the first polypeptide is suppressed or blocked. In preferred embodiments, the transfer of identifying information is suppressed by at least 90%, 95%, 99% or 99.9%. In some embodiments, the transfer of identifying information is completely blocked (not possible); for example, when the modified recording tag is degraded by a nuclease.

In some embodiments, transfer of the identifying information regarding the third binding agent from the third coding tag to the first recording tag or to the modified first recording tag is suppressed or blocked. In some embodiments, the transfer of the identifying information regarding the third binding agent from the third coding tag to the first recording tag is blocked due to absence of the handle or a polynucleotide.

In some embodiments of competitive attenuation of the encoding of a polypeptide which concentration in a sample needs to be adjusted, the polypeptide is contacted with at least two binding agents, wherein the first binding agent comprises a binding moiety capable of binding to the polypeptide and a first coding tag attached to the binding moiety and comprising identifying information regarding the binding agent, whereas the second binding agent comprises the same binding moiety (or binding moiety essentially identical the binding moiety of the first binding agent) and, optionally, a handle attached to the binding moiety and configured to bind, when in close proximity, to the first recording tag associated with the polypeptide. In preferred embodiments, the first and the second binding agents are added to the reaction as a mixture, so they compete for binding to the polypeptide. In some embodiments, the second binding agent comprises the binding moiety and does not comprise the handle. In some embodiments, the second binding agent comprises both the binding moiety and the handle.

In some embodiments, the handle is or comprises a polynucleotide. In some embodiments, the handle binds to the first recording tag by nucleic acid hybridization or via split ligation. Binding of the nucleic acid handle to the first recording tag generates a modified first recording tag comprising an additional spacer sequence at 3′ end via primer extension or ligation. Therefore, on the next steps of the method, following binding of the third binding agent to the first polypeptide that is associated with the modified first recording tag, transfer of identifying information regarding the third binding agent to the modified first recording tag associated with the first polypeptide is suppressed or blocked. In other embodiments, the handle does not comprise a polynucleotide, and transfer of identifying information regarding the third binding agent to the modified first recording tag is suppressed or blocked by a different approach.

In some embodiments, upon binding to the first recording tag, the handle modifies the first recording tag to produce an unfunctional modified first recording tag associated with the first polypeptide.

In some embodiments, the handle comprises a protein enzyme. For example, the handle may comprise a nuclease, a restriction enzyme or a nucleic acid-modifying enzyme that acts locally on first recording tag the due to covalent attachment to the binding moiety of the binding agent, and only following binding of the binding agent to the first polypeptide. The first recording tag becomes unfunctional (modified) after exposure to the handle.

In some embodiments, the handle is or comprises a small chemical moiety capable of reacting with the first recording tag to produce an unfunctional first recording tag associated with the first polypeptide.

In some embodiments, following binding of the binding agent to the first polypeptide, a method to generate a modified (unfunctional) first recording tag is one selected from the following group:
1) Unextendable (3′ OH blocked or deleted) ssDNA ligation (using ssDNA ligase, see Example 8);
2) Unextendable (3′ OH blocked or deleted) dsDNA ligation (using T4 DNA ligase, see Example 9);
3) Unextendable (3′ OH blocked or deleted) nucleotide addition (using TdT, see Example 10);
4) Complementary sequence hybridizing to recoding tag followed by restriction enzyme digestion (see Example 11);
5) Click chemistry reaction between azide group in recording tag and alkyne group in coding tag triggered by copper (see Example 12); and/or
6) Photo-crosslinking between dT in recording tag and a Psoralen moiety in coding tag.

### Exemplary Embodiments

Among the provided embodiments are:

1. A method for analyzing a plurality of different polypeptides from a sample, comprising the steps of:
(a) providing the plurality of different polypeptides and associated recording tags immobilized on a solid support;
(b) contacting the plurality of different polypeptides with a binding agent comprising (i) a binding moiety capable of binding to a first polypeptide from the plurality of different polypeptides; and (ii) a handle attached to the binding moiety and configured to bind, when in close proximity, to a first recording tag associated with the first polypeptide;
(c) following binding of the binding agent to the first polypeptide, providing conditions to allow the handle to bind to the first recording tag and generate a modified first recording tag;
(d) contacting the plurality of different polypeptides with a plurality of binding agents capable of binding to cognate polypeptides from the plurality of different polypeptides, wherein each binding agent from the plurality of binding agents comprises a coding tag that comprises identifying information regarding the binding agent;
(e) following binding of a first binding agent from the plurality of binding agents to the first polypeptide from the plurality of different polypeptides that is associated with the modified first recording tag, and of a second binding agent from the plurality of binding agents to a second polypeptide from the plurality of different polypeptides, providing conditions to allow transfer of identifying information regarding the second binding agent from a second coding tag of the second binding agent to a second recording tag associated with the second polypeptide to generate an extended second recording tag, wherein transfer of identifying information regarding the first binding agent from a first coding tag of the first binding agent to the modified first recording tag associated with the first polypeptide is suppressed or blocked; and
(f) analyzing the extended second recording tag, wherein analyzing comprises a sequencing method, and obtaining the identifying information regarding the second binding agent to provide information regarding the second polypeptide, thereby analyzing the plurality of different polypeptides.

2. The method of embodiment 1, wherein the handle comprises a polynucleotide.

3. The method of embodiment 1, wherein the handle does not comprise a polynucleotide.

4. The method of embodiment 2, wherein the handle binds to the first recording tag by nucleic acid hybridization.

5. The method of any one of embodiments 1-4, wherein the handle comprises a protein enzyme.

6. The method of embodiment 5, wherein the protein enzyme is a nuclease or a restriction enzyme that is configured, upon binding to the first recording tag, to produce an unfunctional first recording tag associated with the first polypeptide.

7. The method of any one of embodiments 1-6, wherein upon binding to the first recording tag, the handle modifies the first recording tag to produce an unfunctional first recording tag associated with the first polypeptide.

8. The method of embodiment 1, wherein following binding of the handle to the first recording tag, the handle remains attached to the first recording tag to form an unfunctional first recording tag associated with the first polypeptide.

9. The method of embodiment 1, wherein the handle comprises a small chemical moiety capable of reacting with the first recording tag to produce an unfunctional first recording tag associated with the first polypeptide.

10. A method for analyzing a plurality of different polypeptides from a sample, comprising the steps of:
(a) providing a first polypeptide from the plurality of different polypeptides and an associated first recording tag immobilized on a solid support;
(b) contacting the first polypeptide with at least two binding agents, wherein a first binding agent of the at least two binding agents comprises: (i) a binding moiety capable of binding to the first polypeptide immobilized on the solid support; and (ii) a first coding tag attached to the binding moiety and comprising identifying information regarding the first binding agent; and a second binding agent of the at least two binding agents comprises (i) the binding moiety essentially identical to the binding moiety of the first binding agent; and, optionally, (ii) a handle attached to the binding moiety of the second binding agent and configured to bind, when in close proximity, to the first recording tag associated with the first polypeptide;
(c) following binding of either the first or second binding agent to the first polypeptide, providing conditions to allow transfer of the identifying information regarding the first binding agent from the first coding tag to the first recording tag by primer extension or ligation to generate an extended first recording tag, or to allow the handle to bind to the first recording tag generating a modified first recording tag;
(d) contacting the first polypeptide with a third binding agent capable of binding to the first polypeptide, wherein the third binding agent comprises a binding moiety capable of binding to the first polypeptide analyte and a third coding tag that comprises identifying information regarding the third binding agent;
(e) following binding of the third binding agent to the first polypeptide, providing conditions to allow transfer of the identifying information regarding the third binding agent from the third coding tag to the extended first recording tag by primer extension or ligation to generate a further extended first recording tag, wherein transfer of the identifying information regarding the third binding agent from the third coding tag to the first recording tag or to the modified first recording tag is suppressed or blocked;
(f) analyzing the further extended recording tag, wherein analyzing comprises a sequencing method, and obtaining the identifying information regarding the first binding agent, and/or the third binding agent to provide information regarding the first polypeptide; and
(g) performing steps (a)-(f) for a second polypeptide from the plurality of different polypeptides, wherein
(i) corresponding first, second and third binding agents for the second polypeptide comprise binding moieties capable of binding to the second polypeptide; and
(ii) a ratio between quantities of the first binding agent and the second binding agent at step (b) is different for the first and the second polypeptides.

11. The method of embodiment 10, wherein the ratio between quantities of the first binding agent and the second binding agent at step (b) is calculated before performing the step (b) based on abundance of the first and the second polypeptides in the sample.

12. The method of embodiment 10 or embodiment 11, wherein the binding moiety of the first binding agent is identical to the binding moiety of the second binding agent.

13. The method of any one of embodiments 10-12, wherein the handle comprises a polynucleotide.

14. The method of embodiment 13, wherein the handle binds to the first recording tag by nucleic acid hybridization.

15. The method of any one of embodiments 10-12, wherein the handle does not comprise a polynucleotide.

16. The method of any one of embodiments 10-12, wherein the handle comprises a protein enzyme.

17. The method of embodiment 16, wherein the protein enzyme is a nuclease or a restriction enzyme that is configured, upon binding to the first recording tag, to produce an unfunctional first recording tag associated with the first polypeptide.

18. The method of any one of embodiments 10-12, wherein upon binding to the first recording tag, the handle modifies the first recording tag to produce an unfunctional first recording tag associated with the first polypeptide.

19. The method of any one of embodiments 10-18, wherein the third binding agent comprises a binding moiety capable of binding to the first or second peptide analyte, and the binding moiety of the third binding agent is capable of binding to one or more N-terminal or C-terminal amino acid residues of the peptide analyte, or capable of binding to the one or more N-terminal or C-terminal amino acid residues modified by a functionalizing reagent.

20. The method of any one of embodiments 10-12, wherein the handle is a small chemical moiety capable of reacting with the first recording tag to produce an unfunctional first recording tag associated with the first polypeptide.

21. The method of embodiment 10 or any one of embodiments 13-20, wherein the second binding agent comprises the binding moiety essentially identical to the binding moiety of the first binding agent and does not comprise the handle.

22. The method of any one of embodiments 10-21, wherein at step (b), the first polypeptide and second polypeptide are contacted at the same time with the same set of binding agents that comprises binding agents capable of binding to the first polypeptide and to the second polypeptide.

23. The method of any one of embodiments 10-22, wherein the first polypeptide and second polypeptide are immobilized on different solid supports.

24. The method of any one of embodiments 10-22, wherein the first polypeptide and second polypeptide are immobilized on the same solid support.

25. A method for analyzing a plurality of different polypeptides immobilized on a support, the method comprising:
(a) contacting the plurality of different polypeptides comprising a first polypeptide and a second polypeptide with a binder, wherein the first polypeptide is associated with a first recording tag and the second polypeptide is associated with a second recording tag, and wherein the binder comprises: (i) a binding moiety capable of binding to the first polypeptide; and (ii) a handle attached to the binding moiety and configured to bind to or react with the first recording tag;
(b) allowing the handle to bind to or react with the first recording tag brought in proximity by binding of the binder to the first polypeptide, thereby modifying the first recording tag to generate a modified first recording tag associated with the first polypeptide;
(c) optionally, fragmenting the plurality of different polypeptides immobilized on the support to generate fragments of different polypeptides immobilized on the support;
(d) contacting the plurality of different polypeptides or the fragments of different polypeptides with a plurality of binding agents, wherein each binding agent comprises: (i) a binding moiety capable of binding to a portion or component of a polypeptide of the plurality of different polypeptides or the fragments thereof; and (ii) a coding tag that comprises identifying information regarding the binding agent;
(e) allowing transfer of identifying information from coding tags of the plurality of binding agents to recording tags associated with the plurality of different polypeptides or the fragments of different polypeptides, thereby generating an extended second recording tag associated with the second polypeptide or fragment thereof upon binding of a binding agent to the second polypeptide or fragment thereof, wherein transfer of identifying information to the modified first recording tag associated with the first polypeptide or fragment thereof is suppressed or blocked; and
(f) analyzing the extended second recording tag to obtain identifying information regarding the binding agent that binds to the second polypeptide or fragment thereof, thereby obtaining information about the second polypeptide or fragment thereof, wherein the analyzing comprises nucleic acid sequencing.

26. The method of embodiment 25, wherein the handle comprises a polynucleotide.

27. The method of embodiment 26, wherein the handle binds to the first recording tag via nucleic acid hybridization.

28. The method of embodiment 25, wherein the handle does not comprise a polynucleotide.

29. The method of any one of embodiments 25-28, wherein the handle comprises a protein, optionally wherein the protein is an enzyme.

30. The method of embodiment 29, wherein the protein is a nuclease that is configured, upon binding to the first recording tag, to produce an unfunctional first recording tag associated with the first polypeptide, optionally wherein the nuclease is a restriction enzyme.

31. The method of any one of embodiments 25-30, wherein upon binding to the first recording tag, the handle modifies the first recording tag to produce an unfunctional first recording tag associated with the first polypeptide.

32. The method of embodiment 31, wherein following binding of the handle to the first recording tag, the handle remains attached to the first recording tag to form an unfunctional first recording tag associated with the first polypeptide.

33. The method of embodiment 31, wherein the handle comprises a chemical moiety capable of reacting with the first recording tag to produce an unfunctional first recording tag associated with the first polypeptide.

34. The method of any one of embodiments 25-33, wherein the plurality of binding agents comprises:
i) a first binding agent comprising a first coding tag that comprises identifying information regarding the first binding agent, wherein the first binding agent is capable of binding to the first polypeptide or fragment thereof, or a component thereof; and
ii) a second binding agent comprising a second coding tag that comprises identifying information regarding the second binding agent, wherein the second binding agent is capable of binding to the second polypeptide or fragment thereof, or a component thereof.

35. The method of embodiment 34, wherein the first binding agent and the second binding agent are the same.

36. The method of embodiment 35, wherein the first binding agent and the second binding agent are different.

37. The method of any one of embodiments 34-36, wherein the transfer of identifying information regarding the second binding agent from the second coding tag to the second recording tag comprises:
(o) generating a double-stranded nucleic acid comprising the second recording tag by (i) joining an end of the second recording tag to an end of the second coding tag, and (ii) optionally, extending the second recording tag using the second coding tag as a template by a polymerase; and
(p) cleaving the double-stranded nucleic acid to generate the extended second recording tag.

38. The method of embodiment 37, wherein the 5′ end of the second recording tag is joined to the 3′ end of the second coding tag by a ligase, with or without gap filling prior to ligation by the ligase.

39. The method of embodiment 38, wherein the second recording tag comprises a hairpin.

40. The method of embodiment 39, wherein the 3′ end of the second recording tag is extended using the second coding tag as the template to generate the double-stranded nucleic acid.

41. The method of any one of embodiments 37-40, wherein the double-stranded nucleic acid is cleaved by a nuclease, optionally wherein the nuclease is a restriction enzyme.

42. The method of any one of embodiments 37-41, wherein the cleavage facilitates release of the second binding agent from the second polypeptide or fragment thereof.

43. The method any one of embodiments 25-42, wherein (d) and (e) are repeated one or more times in sequential cycles.

44. The method of embodiment 43, wherein in a particular cycle of the sequential cycles, the same plurality of binding agents are used compared to a preceding cycle and/or a subsequent cycle.

45. The method of embodiment 43, wherein in a particular cycle of the sequential cycles, a different plurality of binding agents are used compared to a preceding cycle and/or a subsequent cycle.

46. The method of any one of embodiments 43-45, further comprising removing a portion of the second polypeptide or fragment thereof prior to one or more of the sequential cycles of repeating (d) and (e).

47. The method of any one of embodiments 43-46, wherein the first binding agent and/or the second binding agent are capable of binding to a N-terminal amino acid (NTAA) residue or to a NTAA residue functionalized with a modifying reagent.

48. The method of embodiment 47, further comprising removing the N-terminal amino acid (NTAA) of the second polypeptide or fragment thereof to expose a new NTAA of the second polypeptide or fragment thereof prior to one or more of the sequential cycles of repeating (d) and (e).

49. The method of any one of embodiments 1-48, comprising generating a 3′ overhang of the extended second recording tag, wherein the 3′ overhang is available to hybridize with the coding tag of a binding agent of the plurality of binding agent or a different plurality of binding agent.

50. The method of any one of embodiments 25-49, wherein the first polypeptide and the second polypeptide are immobilized on different supports.

51. The method of any one of embodiments 25-49, wherein the first polypeptide and the second polypeptide are immobilized on the same support.

52. The method of any one of embodiments 25-51, wherein the support is a bead.

53. The method of any one of embodiments 25-51, wherein the support is a planar substrate.

54. The method of any one of embodiments 25-53, wherein the first and second polypeptides are of different amino acid sequences, and wherein on the support, molecules of the first polypeptide are more abundant than molecules of the second polypeptide.

55. The method of any one of embodiments 25-53, wherein the first and second polypeptides are of the same amino acid sequences, and wherein on the support, molecules of the first polypeptide comprising a first post-translational modification are more abundant than molecules of the second polypeptide comprising a second post-translational modification.

56. The method of any one of embodiments 25-55, wherein each polypeptide of the plurality of different polypeptides is covalently attached to the support.

57. The method of any one of embodiments 25-56, wherein each polypeptide of the plurality of different polypeptides is covalently attached to an associated recording tag.

58. The method of embodiment 57, wherein the associated recording tag is covalently attached to the support, thereby immobilizing each polypeptide of the plurality of different polypeptides on the support.

59. A method for analyzing molecules of a polypeptide immobilized on a support, the method comprising:
(a) contacting the molecules of the polypeptide with a first binding agent and a second binding agent,
wherein each molecule of the polypeptide is associated with a recording tag immobilized on a support,
wherein the first binding agent comprises (i) a first binding moiety capable of binding to the polypeptide; and (ii) a first coding tag attached to the first binding moiety and comprising identifying information regarding the first binding agent, and
wherein the second binding agent comprises (i) a second binding moiety capable of binding to the polypeptide; and, optionally, (ii) a handle attached to the second binding moiety and configured to bind to or react with the recording tag;
(b) allowing transfer of the identifying information regarding the first binding agent from the first coding tag to the recording tag by primer extension and/or ligation to generate an extended recording tag, and optionally, allowing the handle to bind to or react with the first recording tag to generate a modified first recording tag;
(c) contacting the molecules of the polypeptide with a third binding agent comprising (i) a third binding moiety capable of binding to the polypeptide; and (ii) a third coding tag attached to the third binding moiety and comprising identifying information regarding the third binding agent;
(d) allowing transfer of the identifying information regarding the third binding agent from the third coding tag to the extended recording tag by primer extension and/or ligation to generate a further extended recording tag, wherein transfer of the identifying information regarding the third binding agent from the third coding tag to the recording tag or to the modified first recording tag is suppressed or blocked; and
(e) analyzing the further extended recording tag to obtain identifying information regarding the first binding agent and/or the third binding agent, thereby obtaining information about the polypeptide, wherein the analyzing comprises nucleic acid sequencing.

60. The method of embodiment 59, wherein the polypeptide is a first polypeptide, the method further comprising:

performing (a)-(e) for molecules of a second polypeptide different from the first polypeptide, wherein (i) corresponding first, second and third binding agents for the second polypeptide comprise binding moieties capable of binding to the second polypeptide; and (ii) a ratio between amounts of the first binding agent and the second binding agent is different for the first and the second polypeptides.

61. The method of embodiment 60, wherein molecules of the first polypeptide are more abundant than molecules of the second polypeptide, and the ratio between amounts of the first binding agent and the second binding agent for the first polypeptide is smaller than the ratio for the second polypeptide.

62. The method of embodiment 60 or 61, wherein the ratio for the first polypeptide and/or the ratio for the second polypeptide is selected or determined before contacting the molecules of the first and/or second polypeptides with the corresponding first and second binding agents.

63. The method of embodiment 62, comprising estimating relative abundance of the first and the second polypeptides in a biological sample, wherein the relative abundance correlates with the relative abundance of the first and the second polypeptides immobilized on supports.

64. The method of any one of embodiments 59-63, wherein for the first polypeptide and/or for the second polypeptide, the binding moiety of the first binding agent is essentially identical to the binding moiety of the second binding agent.

65. The method of any one of embodiments 60-64, wherein the first polypeptide and second polypeptide are immobilized on the same support

### EXAMPLES

The following examples are offered to illustrate but not to limit the methods, compositions, and uses provided herein. Certain aspects of the present disclosure, including, but not limited to, embodiments for the Proteocode™ polypeptide sequencing assay, information transfer between coding tags and recording tags, methods of making nucleotide-polypeptide conjugates, methods for attachment of nucleotide-polypeptide conjugates to a support, methods of generating barcodes, methods of generating specific binding agents recognizing an N-terminal amino acid of a polypeptide, reagents and methods for modifying and/or removing an N-terminal amino acid from a polypeptide, methods for analyzing extended recording tags were disclosed in earlier published application, e.g., US 2019/0145982 A1, US 2020/0348308 A1, US 2020/0348307 A1, US 2021/0208150 A1, US 2022/0049246 A1, U.S. Pat. No. 11,427,814, US 2022/0227889 A1, US 2022/0283175 A1, the contents of which are incorporated herein by reference in its entirety.

### Example 1. Immobilization of Recording Tag-Labeled Polypeptides to a Solid Support

Recording tag-labeled polypeptides are immobilized on a support via an IEDDA click chemistry reaction using an mTet group on the recording tag and a TCO group on the surface of activated beads (solid support). 200 ng of M-270 TCO beads are resuspended in 100 μl phosphate coupling buffer. 5 pmol of DNA recording tag labeled peptides comprising an mTet moiety on the recording tag is added to the beads for a final concentration of 50 nM. The reaction is incubated for 1 hr at room temperature. After immobilization, unreacted TCO groups on the support are quenched with 1 mM methyl tetrazine acid in phosphate coupling buffer for 1 hr at room temperature.

Magnetic beads suitable for click-chemistry immobilization are created by converting M-270 amine magnetic Dynabeads (Thermo Fisher, USA) to either azide or TCO-derivatized beads capable of coupling to alkyne or methyl Tetrazine-labeled oligo-peptide conjugates, respectively (see also Examples 20-21 of US 2019/0145982 A1). Namely, 10 mg of M-270 beads are washed and resuspended in 500 μl borate buffer (100 mM sodium borate, pH 8.5). A mixture of TCO-PEG (12-120)-NHS (Nanocs) and methyl-PEG (12-120)-NHS is resuspended at 1 mM in DMSO and incubated with M-270 amine beads at room temperature overnight. The ratio of the Methyl to TCO PEG is titrated to adjust the final TCO surface density on the beads such that there is <100 TCO moieties/um2. Unreacted amine groups are capped with a mixture of 0.1M acetic anhydride and 0.1M DIEA in DMF (500 μl for 10 mg of beads) at room temperature for 2 hrs. After capping and washing 3× in DMF, the beads are resuspended in phosphate coupling buffer at 10 mg/ml.

### Example 2. Polypeptide Immobilization Using Nucleic Acid Hybridization and Joining to a Solid Support

This example describes exemplary methods for joining (immobilizing) nucleic acid-polypeptide conjugates, such as conjugates of a polypeptide with recording tag, to a solid support. In a hybridization based method of immobilization, nucleic acid-polypeptide conjugates were hybridized and ligated to short hairpin capture DNAs that were chemically immobilized on beads (NHS-Activated Sepharose™ High Performance, Cytiva, USA). The capture DNAs were conjugated to the beads using trans-cyclooctene (TCO) and methyltetrazine (mTet)-based click chemistry. TCO-modified short hairpin capture DNAs (16 basepair stem, 4 base loop, 17 base 5′ overhang) were reacted with mTet-coated beads. Phosphorylated nucleic acid-polypeptide conjugates (20 nM) were annealed to the hairpin DNAs attached to beads in 0.5 M NaCl, 50 mM sodium citrate, 0.02% SDS, pH 7.0, and incubated for 30 minutes at 37° C. The beads were washed once with PBST (1× phosphate buffer, 0.1% Tween-20) and resuspended in 1× Quick ligation solution (New England Biolabs, USA) with T4 DNA ligase. After a 30 min incubation at 25° C., the beads were washed once with PBST, three times with 0.1 M NaOH, 0.1% Tween-20, three times with 1× phosphate buffer, 0.1% Tween-20, and resuspended in 50 μL of PBST.

The total immobilized nucleic acid-polypeptide conjugates including amino FA-terminal peptides (FAGVAMPGAEDDVVGSGSK; SEQ ID NO: 3), amino AFA-terminal peptides (AFAGVAMPGAEDDVVGSGSK; SEQ ID NO: 4), and an amino AA-terminal peptides (AAGVAMPGAEDDVVGSGSK; SEQ ID NO: 5) were quantified by qPCR using specific primer sets. For comparison, peptides were immobilized onto beads using a non-hybridization based method that did not involve a ligation step. The non-hybridization based method was performed by incubating 30 μM TCO-modified DNA-tagged peptides including amino FA-terminal peptides, amino AFA-terminal peptides, and amino AA-terminal peptides, with mTet-coated magnetic beads overnight at 25° C.

As shown in Table 1, similar Ct values were observed in the non-hybridization preparation method with 1:100,000 grafting density and the hybridization based preparation method with 1:10,000 grafting density. Loading amount of DNA-tagged peptides for the hybridization based preparation method was 1/3000 compared to that for the non-hybridization preparation method. In general, it was observed that less starting material was needed for the hybridization based immobilization method.

TABLE 1
Comparison of loading hybridization and
non-hybridization immobilization methods.

| | Non-hybridization based | Hybridization based |
|---|---|---|
| | immobilization method | immobilization method |
| Grafting:Passivation | (−Ligation) | (+Ligation) |
| 1:100,000 | 19.4 | 25.4 |
| 1:10,000 | — | 21.1 |

### Example 3. Preparation of Polypeptides from Biological Samples

In one example, polypeptides used in the encoding methods are obtained by processing a biological sample, such as a cell lysate or a plasma sample. There are a wide variety of protocols known in the art for making protein lysates from various sample types. Most variations on the protocol depend on cell type and whether the extracted proteins in the lysate are to be analyzed in a non-denatured or denatured state. For the disclosed methods, either native conformation or denatured proteins can be immobilized to a solid support. Moreover, after immobilization of native proteins, the proteins immobilized on the solid support can be denatured.

Examples of non-denaturing protein lysis buffers include: RPPA buffer consisting of 50 mm HEPES (pH 7.4), 150 mM NaCl, 1% Triton X-100, 1.5 mM MgCl2, 10% glycerol; and commercial buffers such as M-PER mammalian protein extraction reagent (Thermo-Fisher). A denaturing lysis buffer comprises 50 mm HEPES (pH 8), 1% SDS. The addition of Urea (1M-3M) or Guanidine HCl (1-8M) can also be used in denaturing the protein sample. In addition to the above components of lysis buffers, protease and phosphatase inhibitors are also generally included. Examples of protease inhibitors and typical concentrations include aptrotinin (2 μg/ml), leupeptin (5-10 μg/ml), benzamidine (15 μg/ml), pepstatin A (1 μg/ml), PMSF (1 mM), EDTA (5 mM), and EGTA (1 mM). Examples of phosphatase inhibitors include Na pyrophosphate (10 mM), sodium fluoride (5-100 mM) and sodium orthovanadate (1 mM). Additional additives can include DNAaseI to remove DNA from the protein sample, and reducing agents such as DTT to reduce disulfide bonds.

An example of a non-denaturing protein lysate protocol prepared from tissue culture cells is as follows: Adherent cells are trypsinized (0.05% trypsin-EDTA in PBS), collected by centrifugation (200 g for 5 min.), and washed 2× in ice cold PBS. Ice-cold M-PER mammalian extraction reagent (˜1 mL per 107 cells/100 mm dish or 150 cm2 flask) supplemented with protease/phosphatase inhibitors and additives (e.g., EDTA free complete inhibitors (Roche) and PhosStop (Roche) is added. The resulting cell suspension is incubated on a rotating shaker at 4° C. for 20 min. and then centrifuged at 4° C. at 12,000 rpm (depending on cell type) for 20 min to isolate the protein supernatant. The protein is quantitated using the BCA assay, and resuspended at 1 mg/ml in PBS. The protein lysates can be used immediately or snap frozen in liquid nitrogen and stored at −80° C.

An example of a denaturing protein lysate protocol, based on the SP3 protocol of Hughs et al., prepared from tissue culture cells is as follows: adherent cells are trypsinized (0.05% trypsin-EDTA in PBS), collected by centrifugation (200 g for 5 min.), and washed 2× in ice cold PBS. Ice-cold denaturing lysis buffer (˜1 mL per 107 cells/100 mm dish or 150 cm2 flask) supplemented with protease/phosphatase inhibitors and additives (e.g., 1× complete Protease Inhibitor Cocktail (Roche)) is added. The resulting cell suspension is incubated at 95° C. for 5 min and placed on ice for 5 min. Benzonase Nuclease (500 U/ml) is added to the lysate and incubated at 37° C. for 30 min to remove DNA and RNA. The proteins are reduced by addition of 5 μL of 200 mM DTT per 100 μL of lysate and incubated for 45° C. for 30 min. Alklylation of protein cysteine groups is accomplished by addition of 10 μL of 400 mM iodoacetamide per 100 μL of lysate and incubated in the dark at 24° C. for 30 min. Reactions are quenched by addition of 10 μL of 200 mM DTT per 100 μL of lysate. Proteins are optionally acylated by adding 2 μl an acid anhydride and 100 μl of 1 M Na2CO3 (pH 8.5) per 100 μl of lysate. Incubate for 30 min at room temp. Valeric, benzoic, and proprionic anhydride are recommended rather than acetic anhydride to enable “in vivo” acetylated lysines to be distinguished from “in situ” blocking of lysine groups by acylation (Sidoli, Yuan et al. 2015). The reaction is quenched by addition of 5 mg of Tris(2-aminoethyl)amine, polymer (Sigma) and incubation at room temperature for 30 min. Polymer resin is removed by centrifuging lysate at 2000 g for 1 min through a 0.45 um cellulose acetate Spin-X tube (Corning). The protein is quantitated using the BCA assay, and resuspended at 1 mg/ml in PBS.

### Example 4. Polypeptide Sample Preparation Workflow for the Encoding Assay

This example demonstrates an exemplary sample preparation workflow used for preparing peptide-recording tag conjugates and immobilizing them on a solid support.

Protein denaturation and digestion. For a 10 μg of protein sample, samples were diluted to the desired protein input concentration in an appropriate buffer (10 μg/45 μL; 100 mM carbonate/bicarbonate buffer at pH 9.15 with 0.1% sodium dodecyl sulfate (SDS)). Cysteines were reduced with TCEP added to a final concentration of 5 mM. Samples were incubated for 15 min at 37° C., and, after cooling, iodoacetamide (IAA) stock was added to a final concentration of 20 mM. Samples were incubated at 37° C. for 15 min to allow the alkylation to proceed. Lysine side chains were blocked by addition of NHS-acetate (ARR1, 10 mM) at 60° C. for 30 min. Trypsin was added at a 1:25 ratio, by mass, for each sample and incubated for 2 hours at 37° C. to digest the sample. Resulting peptides were then functionalized at the amine terminus using 10 mM photocleavable linker (AAR2, a self-immolative linker comprising para-nitrophenyl carbonate reactive ester coupled to a para-nitrobenzylcarbonate and a PEG-mTET enrichment tag) at 37° C. for 60 min.

Peptide immobilization to solid support. Peptides were immobilized to a solid support (TCO agarose, Click Chemistry Tools) through the enrichment tag (mTET moiety). The peptide mixture was incubated with 130 μL TCO beads for 60 min at 37° C. to immobilize the modified peptides. Other combinations of enrichment tag and compatible solid support can be implemented. Excess material (e.g., cellular components), unreacted peptides, and reaction components were removed by washing three times with PBS-T (PBS (phosphate-buffered saline) plus 0.1% TWEEN® 20).

CHD functionalization of C-terminal arginines and polypeptide-DNA conjugate formation. Each sample was resuspended after concentration in vacuo in 20 μL 0.2 M NaOH (pH 13.7), 1 M KPhos (pH 8.3), or 2 M KPhos (pH 8.3). CHD Stock (CHD-PEG₃-azide in DMSO) was added for a final concentration of 10 mM and incubated at 37° C. for 1 hr, 80° C. for 1.5 hours, or 80° C. for 1 hour, respectively. The reaction was neutralized by adding equal volume 1 M Tris, pH 7.4, and washed to remove excess/unreacted CHD-PEG₃-azide and impurities. Samples were diluted to 10 μg/1000 μL in PBS-T. On-bead DNA-polypeptide conjugate (polypeptide-conjugation reagent-nucleic acid conjugate) formation was carried out using a DBCO-DNA (Dibenzocyclooctyne-coupled DNA; DNA=5′-/5Phos/CAA GTT CTC AGT AAT GCG TAG/DBCOdT/CC GCG ACA CTA G-3′; SEQ ID NO: 6) and incubating for 16 hours. The beads containing the conjugated product were washed to remove excess DBCO-DNA.

Further processing of peptide-DNA conjugates. Upon completion of incubation, beads were centrifuged and washed to remove any excess DBCO-DNA. Sample barcodes were added and beads were washed twice with 200 μL PBS-T. The peptide-DNA chimera was eluted with 10 μL 4 mM biotin, 20 mM Tris-HCl, and 50 mM NaCl. Chimera formation and barcoding were confirmed by loading 0.5 μL of sample (5 pmol) on TBU gel electrophoresis. (15% TBU gel, 200V, 50 min). The peptides were then immobilized on a solid support. The DNA of the peptide-DNA chimera was hybridized and ligated to a DNA recording tag containing a complementary sequence attached to beads at appropriate spacing and density (see Example 2 and US patent publication US 2022/0049246 A1, incorporated herein).

### Example 5. Exemplary Multicycle Encoding Assay

In this example, polypeptides were immobilized on a solid support as in Example 2. For the encoding assay described in this Example, a cognate test peptide, FSGVAMPGAEDDVVGSGSK (azide) (SEQ ID NO: 7), and a non-cognate test peptide, AFSGVAMPGAEDDVVGSGSK (azide) (SEQ ID NO: 8), conjugated to DNA recording tags (/5Phos/CGACGCTCT/iAmMC6T/CCGATCTNNNTTGTCACACTAC, SEQ ID NO: 9, and/5Phos/CGACGCTCT/iAmMC6T/CCGATCTNNNAGGACACACTAC, SEQ ID NO: 10, respectively) were used. The recording tag-polypeptide conjugates were joined to immobilized bead-attached capture DNA (CACTCAGTCCATTAACNNNNNNNNNNCTAGTGTCGCGGACTACGCATTACTGAGA AGCTTGCTAGTCGACGTGGTCCTTTTGGACCACGTCGACTAG, SEQ ID NO: 11) essentially as described in Example 2. DNA-polypeptide conjugates (20 nM) were annealed to the capture DNAs attached to beads in 5×SSC, 0.02% SDS, and incubated for 30 minutes at 37° C. The beads were washed once with PBST and resuspended in 1× Quick ligation solution (New England Biolabs, USA) with T4 DNA ligase. After a 30-minute incubation at 25° C., and the beads were washed with PBST, two times of 0.1M NaOH+0.1% Tween-20 and twice of PBST.

The exemplary binding agent, an engineered F-binder which has affinity specifically to N-terminal phenylalanine residues of polypeptides, was used in this example to recognize the target conjugates. The F-binder was engineered from ClpS2 (obtained by Phage display library screening as disclosed in US 2021/0208150 A1). The F-binder was expressed as a fusion with SpyCatcher protein, and the fusion was reacted to SpyTag-coding tag fusion (the coding tag was attached to SpyTag via a PEG linker, as described in US 2021/0208150 A1) leading to the F-binder-coding tag conjugate, used further during the encoding assay. The coding tags used in the assay comprised barcodes containing identifying information for the F-binder, and coding tags were different for each cycle of the assay.

Each encoding cycle of the encoding assay described in this example consists of contacting the immobilized peptides with the F-binder-coding tag conjugate, followed by transferring information of the coding tag to the recording tags associated with the peptides by a primer extension reaction after partial hybridization between the coding tag and the recording tag through a shared spacer region using a DNA polymerase having 5′-to-3′ polymerization activity and having substantially reduced 3′-to-5′ exonuclease activity. After the recording tag extension, the F-binder-coding tag conjugate is washed away, and the immobilized peptides with associated extended recording tags are ready for the next cycle of encoding. In the encoding assays of this and the following Examples, the same F-binder is used in each encoding cycle, but conjugated with cycle-specific coding tags, so a different binder coding tag conjugate is used in each encoding cycle. The cycle-specific coding tags allow to evaluate efficiency of encoding (information transfer) during each encoding cycle by analyzing extended recording tag sequences.

In this Example, multicycle encoding reactions were carried out using Klenow Fragment exo-enzyme mutants to perform the primer extension reaction. The efficiencies of the encoding reactions were evaluated based on yield (based on fractions of recording tag reads encoded) and background signal (fractions of recording tag reads encoded generated on the non-cognate peptide). Under optimal conditions, the coding tag and the recording tag form hybridization complex via hybridization of the corresponding spacer regions mostly when the F-binder binds to the cognate peptide (which has F at the N-terminus). During each cycle the recording tag incorporates the coding tag barcode if the extension reaction occurs. Sequencing of recording tags after each cycle is used to estimate the fraction of recording tags being extended (encoded). If the spacer region length is longer than optimal, hybridization between the coding tag and the recording tag of a non-cognate peptide can occur even without the binding of the F-binder to the peptide, thus creating a non-specific signal and making essentially impossible to identify the component of the peptide to which the binding agent has affinity. In this experiment, both specific (generated from the cognate peptide) and non-specific (generated from the non-cognate peptide) signals were assessed after each encoding cycle.

The F-binder with the coding tag (50 nM) was incubated with the recording tag-peptide conjugates immobilized on the beads for 15 min at 25° C., followed by washing with (500 mM NaCl, 3 mM Na₂HPO₄, 1.1 mM KH2PO₃ and 0.1% Tween-20, pH 7.4). The beads were incubation with 125 nM KF exo-wild type or its mutants for 10 min at 25° C. in presence of 50 nM cycle cap oligo (for the extension of non-encoded DNA), dNTPs (each at 125 uM), 50 mM Tris-HCl (pH, 7.5), 2 mM MgSO₄, 50 mM NaCl, 1 mM DTT, 0.1% Tween-20, and 0.1 mg/mL BSA. The samples were washed with either PBST, 0.1 M NaOH+0.1% Tween-20, and PBST at 25 C (for 2 cycle encoding); or PBST, PBST+30% formamide at 50° C. for 5 min, and PBST (for 5 cycle encoding).

After the encoding cycles, the sample were finally introduced with primer binding site for PCR and NGS by incubation of 400 uM of an capping oligo (GAAGAGTAATTAGATCGGAAGAGCACACGTCTGAACTCGACTGGAGTTCAGACGT GTGCTCTTCCGATCTAATTACTCTTCTAGAGATGG/3SpC3/(for 2 cycle encoding, SEQ ID NO: 12) with 0.125 U/uL of WT Klenow fragment (3′->5′ exo-), dNTPs (each at 125 uM), 50 mM Tris-HCl (pH, 7.5), 2 mM MgSO₄, 50 mM NaCl, 1 mM DTT, 0.1% Tween-20, and 0.1 mg/mL BSA at 25° C. for 10 min.

When wild type Klenow Fragment exo-enzyme was used during primer extension, a high non-specific signal generated from the non-cognate peptide was observed starting from the second cycle (FIG. 8). The reason for this non-specific signal was an increase in the spacer region length after primer extension due to significant template independent nucleotide addition, which effectively adds an extra “A” (A-tailing) to the spacer region. Eliminating A-tailing by utilizing the KF exo-mutants R754L, R754Y or R754H generated much less non-specific signal during multi-cycle encoding, while preserving the specific signal generated from the cognate peptide. The ratio between the specific signal and non-specific signal produced during multi-cycle encoding was at least 5 or more (FIG. 8).

### Example 6. Capping Abundant Polypeptides During the Encoding Assay to Adjust Signal Generated from them

Polypeptides isolated from biological samples (e.g., plasma, cells, tissues) are chemically linked to hairpin capture DNA immobilized on beads (see Example 2). Each capture DNA contains a recording tag with spacer A (SP_A) at the 3′ end (FIGS. 3A-3B). Binding agents (e.g., antibody, chemicals, proteins) conjugated to coding tag comprising complementary sequence to spacer A (SP_A′) and spacer B (SP_B′) are added to the beads to bind to polypeptides targeted to depletion. After washing unbound binding agent-coding tag conjugates, an enzyme (e.g., polymerase, ligase, restriction enzyme) or a chemical (e.g., copper for click chemistry) is added to modify the 3′ end of recording tag conjugated to the polypeptide bound by the binding agent and generate the unfunctional recording tag. The beads are washed and treated with trypsin to digest polypeptides for sequencing. At the first cycle of polypeptide sequencing, a plurality of binding agents specific for N-terminal amino acid residues bind to N-termini of polypeptides, and the binding agent's identifying information is transferred only to the recording tag comprising the unmodified 3′ end spacer A (SP_A) by using an enzyme (DNA polymerase or ligase) addition. As a result, the encoding reaction for the immobilized polypeptides successfully proceeds further only on the unmodified recording tags associated with the polypeptides.

Specific experimental conditions for modification methods of recording tags of polypeptides targeted to depletion are disclosed in the following Examples 7-14 below.

### Example 7. Modification by Extension

Recording tag containing 5′ phosphorylation, DBCO modification at internal position and spacer A (SP_A) sequence at 3′ end is hybridized to hairpin DNA immobilized on Sepharose™ beads by incubation at 37° C. for 30 min, washed once with PBST, and ligated by incubation with 150 μL of ligation mixture containing 66 mM Tris-HCl, 10 mM MgCl₂, 1 mM DTT, 1 mM ATP, 7.5% PEG 6000, 1 L of Quick ligase (NEB) at 25° C. for 30 min. The DBCO-modified recording tag beads are washed three times with 150 μL of PBST (PBS+0.1% Tween-20). On the other hand, proteins/peptides mixture in biological samples (e.g. plasma, cells, tissues) are incubated with azide-PEG₄-NHS ester, and the azide-modified protein/peptides are purified using 7K MWCO Zeba Spin Desalting Columns (Thermo Fisher). The azide-modified protein/peptides mixture is incubated with DBCO-modified recording tag beads at 37° C. overnight to immobilize protein/peptide on recording tag beads. The proteins/peptides immobilized recording tag beads are washed three times with 150 μL PBST for assay.

First cycle binding agents (e.g., antibody, chemicals, proteins) are used to encode depletion-target proteins and peptides. The proteins/peptides immobilized recording tag beads containing a recording tag with spacer A (SP_A) at the 3′ end are incubated with 150 μL of 50 nM binding agents conjugated to coding tag containing complementary sequences to spacer A (SP_A′) and spacer B (SP_B′) in PBST at 25° C. for 30 minutes. Unbound binding agent-coding tag conjugates are washed twice with 150 μL of PBST+500 mM NaCl, and then incubated with the 150 μL of encoding mixture containing 0.125 U/μL Klenow fragment (3′->5′ exo-), dNTP mixture (125 μM for each), 50 mM Tris-HCl (pH, 7.5), 2 mM MgSO₄, 50 mM NaCl, 1 mM DTT. 0.1% Tween-20, and 0.1 mg/mL BSA at 25° C. for 5 minutes to copy the spacer B to the recording tag with depletion-target. The beads are washed three times with 150 μL of PBST. As a result, the recording tags with depletion-target are installed with spacer B, and other recording tags keep spacer A.

After washing three times with 150 μL of PBST and 150 μL of PBS, the beads are resuspended in 200 μL of 10 mM ammonium bicarbonate, pH 9, and 2 μL of 5 mM TCEP is added and incubated at 37° C. for 30 minutes for reduction. The iodoacetamide is added to the beads mixture to adjust to 20 mM and incubated at 37° C. for 30 minutes for cysteine alkylation. After washing three times with 150 μL of PBST, the beads are incubated with 150 μL of acetylation reaction solution including 50 mM Ac-NHS, 50 mM carbonate/bicarbonate, 4 M Urea at 60° C. for 60 minutes. The trypsin is added to the beads mixture with 150 μL of Tris-HCl, pH 8.0 to adjust to 0.4 mg/mL and incubated at 37° C. overnight for proteins/peptides digestion. The trypsinized beads are washed three times with 150 μL of PBST for protein sequencing.

For protein sequencing, N-terminus specific binding agents are used to identify the amino acids at N-terminus of peptides on beads. The trypsinized peptides immobilized recording tag beads are incubated with 150 μL of 50 nM binding agents conjugated to coding tag containing and complementary sequences to spacer A (SP_A′) and spacer C (SP_C′) in PBST at 25° C. for 15 minutes. Unbound binding agent-coding tag conjugates are washed twice with 150 μL of PBST+500 mM NaCl, and then incubated with the 150 μL of encoding mixture containing 0.125 U/μL Klenow fragment (3′->5′ exo-), dNTP mixture (125 μM for each), 50 mM Tris-HCl (pH, 7.5), 2 mM MgSO₄, 50 mM NaCl, 1 mM DTT, 0.1% Tween-20, 0.1 mg/mL BSA and cycle capping oligo SpA_SpC at 25° C. for 5 minutes to copy the binding agent barcode and the spacer C to the recording tag containing spacer A at the 3′ end. The beads are washed three times with 150 μL of PBST. As a result, recording tags other than depletion-target are installed with spacer C for additional protein sequencing cycles.

After multicycle steps for protein sequencing, the recording tags containing protein sequence information are capped by NGS adaptor sequences, followed by PCR for next generation sequencing.

### Example 8. Modification by ssDNA Ligation

Recording tag containing 5′ phosphorylated, DBCO modification at internal position and spacer A (SP_A) sequence at 3′ end is hybridized to hairpin DNA immobilized on Sepharose™ beads by incubation at 37° C. for 30 min, washed once with PBST, and ligated by incubation with 150 μL of ligation mixture containing 66 mM Tris-HCl, 10 mM MgCl₂, 1 mM DTT, 1 mM ATP, 7.5% PEG 6000, 1 μL of Quick ligase (NEB) at 25° C. for 30 min. The DBCO-modified recording tag beads are washed three times with 150 μL of PBST (PBS+0.1% Tween-20). On the other hand, proteins/peptides mixture in biological samples (e.g., plasma, cells, tissues) are incubated with azide-PEG₄-NHS ester, and the azide-modified protein/peptides are purified using 7K MWCO Zeba Spin Desalting Columns (Thermo Fisher). The azide-modified protein/peptides mixture is incubated with DBCO-modified recording tag beads at 37° C. overnight to immobilize protein/peptide on recording tag beads. The proteins/peptides immobilized recording tag beads are washed three times with 150 μL PBST for assay.

First cycle binding agents (e.g., antibody, chemicals, proteins) are used to block depletion-target proteins and peptides. The proteins/peptides immobilized recording tag beads containing a recording tag with spacer A (SP_A) at the 3′ end are incubated with 150 μL of 50 nM binding agents conjugated to coding tag containing 5′ phosphorylated and 3′ C₃ blocked single strand short DNA sequence in PBST at 25° C. for 30 minutes. Unbound binding agent-coding tag conjugates are washed twice with 150 μL of PBST+500 mM NaCl, and then incubated with the 150 μL of ssDNA ligation mixture containing 33 mM Tris-acetate (pH 7.5), 66 mM potassium acetate, 0.5 mM DTT, 2.5 mM MnCl₂, 1 M Betaine and 5 U/μL CircLigase II ssDNA Ligase (Lucigen) at 60° C. for 1 hour to block the 3′ end of recording tag with depletion-target. The beads are washed three times with 150 μL of PBST. As a result, the 3′ end of recording tags with depletion-target are blocked, resulting in unextendable ends, and other recording tags keep extendable spacer A.

After washing three times with 150 μL of PBST and 150 μL of PBS, the beads are resuspended in 200 μL of 10 mM ammonium bicarbonate, pH 9, and 2 μL of 5 mM TCEP is added and incubated at 37° C. for 30 minutes for reduction. The iodoacetamide is added to the beads mixture to adjust to 20 mM and incubated at 37° C. for 30 minutes for alkylation. After washing three times with 150 μL of PBST, the beads are incubated with 150 μL of acetylation reaction solution including 50 mM Ac-NHS, 50 mM carbonate/bicarbonate, 4 M Urca at 60° C. for 60 minutes. The trypsin is added to the beads mixture with 150 μL of Tris-HCl. pH 8.0 to adjust to 0.4 mg/mL and incubated at 37° C. overnight for proteins/peptides digestion. The trypsinized beads are washed three times with 150 μL of PBST for protein sequencing.

For protein sequencing, N-terminus specific binding agents are used to identify the amino acids at N-terminus of peptides on beads. The trypsinized peptides immobilized recording tag beads are incubated with 150 μL of 50 nM binding agents conjugated to coding tag containing and complementary sequences to spacer A (SP_A′) and spacer C (SP_C′) in PBST at 25° C. for 15 minutes. Unbound binding agent-coding tag conjugates are washed twice with 150 μL of PBST+500 mM NaCl, and then incubated with the 150 μL of encoding mixture containing 0.125 U/μL Klenow fragment (3′->5′ exo-), dNTP mixture (125 μM for each), 50 mM Tris-HCl (pH, 7.5), 2 mM MgSO₄, 50 mM NaCl, 1 mM DTT, 0.1% Tween-20, 0.1 mg/mL BSA and cycle capping oligo SpA_SpC at 25° C. for 5 minutes to copy the binding agent barcode and the spacer C to the recording tag containing extendable spacer A at the 3′ end. The beads are washed three times with 150 μL of PBST. As a result, recording tags other than depletion-target are installed with spacer C for additional protein sequencing cycles.

After multicycle steps for protein sequencing, the recording tags containing protein sequence information are capped by NGS adaptor sequences, followed by PCR for next generation sequencing.

### Example 9. Modification by dsDNA Ligation

Recording tag containing 5′ phosphorylated, DBCO modification at internal position and spacer A (SP_A) sequence at 3′ end is hybridized to hairpin DNA immobilized on Sepharose™ beads by incubation at 37° C. for 30 min, washed once with PBST, and ligated by incubation with 150 μL of ligation mixture containing 66 mM Tris-HCl, 10 mM MgCl₂, 1 mM DTT, 1 mM ATP, 7.5% PEG 6000, 1 μL of Quick ligase (NEB) at 25° C. for 30 min. The DBCO-modified recording tag beads are washed three times with 150 μL of PBST (PBS+0.1% Tween-20). On the other hand, proteins/peptides mixture in biological samples (e.g., plasma, cells, tissues) are incubated with azide-PEG₄-NHS ester, and the azide-modified protein/peptides are purified using 7K MWCO Zeba Spin Desalting Columns (Thermo Fisher). The azide-modified protein/peptides mixture is incubated with DBCO-modified recording tag beads at 37° C. overnight to immobilize protein/peptide on recording tag beads. The proteins/peptides immobilized recording tag beads are washed three times with 150 μL PBST for assay.

First cycle binding agents (e.g., antibody, chemicals, proteins) are used to block depletion-target proteins and peptides. The proteins/peptides immobilized recording tag beads containing a recording tag with spacer A (SP_A) at the 3′ end are incubated with 150 μL of 50 nM binding agents conjugated to coding tag containing 5′ phosphorylated and 3′ C₃ blocked double strand DNA with 3′ short overhang sequences to spacer A (SP_A′) in PBST at 25° C. for 30 minutes. Unbound binding agent-coding tag conjugates are washed twice with 150 μL of PBST+500 mM NaCl, and then incubated with the 150 μL of dsDNA ligation mixture containing 66 mM Tris-HCl, 10 mM MgCl₂, 1 mM DTT, 1 mM ATP, 7.5% PEG6000, 1 μL of Quick ligase (NEB) at 25° C. for 5 minutes to block the 3′ end of recording tag with depletion-target. The beads are washed three times with 150 μL of PBST. As a result, the 3′ end of recording tags with depletion-target are blocked, resulting in unextendable ends, and other recording tags keep extendable spacer A.

After washing three times with 150 μL of PBST and 150 μL of PBS, the beads are resuspended in 200 μL of 10 mM ammonium bicarbonate, pH 9, and 2 μL of 5 mM TCEP is added and incubated at 37° C. for 30 minutes for reduction. The iodoacetamide is added to the beads mixture to adjust to 20 mM and incubated at 37° C. for 30 minutes for cysteine alkylation. After washing three times with 150 μL of PBST, the beads are incubated with 150 μL of acetylation reaction solution including 50 mM Ac-NHS, 50 mM carbonate/bicarbonate, 4 M Urea at 60° C. for 60 minutes. The trypsin is added to the beads mixture with 150 μL of Tris-HCl, pH 8.0 to adjust to 0.4 mg/mL and incubated at 37° C. overnight for proteins/peptides digestion. The trypsinized beads are washed three times with 150 μL of PBST for protein sequencing.

For protein sequencing, N-terminus specific binding agents are used to identify the amino acids at N-terminus of peptides on beads. The trypsinized peptides immobilized recording tag beads are incubated with 150 μL of 50 nM binding agents conjugated to coding tag containing and complementary sequences to spacer A (SP_A′) and spacer C (SP_C′) in PBST at 25° C. for 15 minutes. Unbound binding agent-coding tag conjugates are washed twice with 150 μL of PBST+500 mM NaCl, and then incubated with the 150 μL of encoding mixture containing 0.125 U/μL Klenow fragment (3′->5′ exo-), dNTP mixture (125 μM for each), 50 mM Tris-HCl (pH, 7.5), 2 mM MgSO₄, 50 mM NaCl, 1 mM DTT, 0.1% Tween-20, 0.1 mg/mL BSA and cycle capping oligo SpA_SpC at 25° C. for 5 minutes to copy the binding agent barcode and the spacer C to the recording tag containing extendable spacer A at the 3′ end. The beads are washed three times with 150 μL of PBST. As a result, recording tags other than depletion-target are installed with spacer C for additional protein sequencing cycles.

After multicycle steps for protein sequencing, the recording tags containing protein sequence information are capped by NGS adaptor sequences, followed by PCR for next generation sequencing.

### Example 10. Modification by Unextendible Nucleotide Addition

Recording tag containing 5′ phosphorylated, DBCO modification at internal position and spacer A (SP_A) sequence at 3′ end is hybridized to hairpin DNA immobilized on Sepharose™ beads by incubation at 37° C. for 30 min, washed once with PBST, and ligated by incubation with 150 μL of ligation mixture containing 66 mM Tris-HCl, 10 mM MgCl₂, 1 mM DTT, 1 mM ATP, 7.5% PEG 6000, 1 μL of Quick ligase (NEB) at 25° C. for 30 min. The DBCO-modified recording tag beads are washed three times with 150 μL of PBST (PBS+0.1% Tween-20). On the other hand, proteins/peptides mixture in biological samples (e.g., plasma, cells, tissues) are incubated with azide-PEG₄-NHS ester, and the azide-modified protein/peptides are purified using 7K MWCO Zeba Spin Desalting Columns (Thermo Fisher). The azide-modified protein/peptides mixture is incubated with DBCO-modified recording tag beads at 37° C. overnight to immobilize protein/peptide on recording tag beads. The proteins/peptides immobilized recording tag beads are washed three times with 150 μL PBST for assay.

First cycle binding agents (e.g., antibody, chemicals, proteins) are used to block depletion-target proteins and peptides. The proteins/peptides immobilized recording tag beads containing a recording tag with spacer A (SP_A) at the 3′ end are incubated with 150 μL of 50 nM binding agents conjugated to ddTTP via 3′ PEG linker in PBST at 25° C. for 30 minutes. Unbound binding agent-coding tag conjugates are washed twice with 150 μL of PBST+500 mM NaCl, and then incubated with the 150 μL of single nucleotide extension mixture containing 50 mM potassium acetate, 20 mM Tris-acetate, 10 mM magnesium acetate, pH 7.9, 0.25 mM CoCl₂ and 20 U/μL of Terminal Transferase (NEB) at 37° C. for 30 minutes to block the 3′ end of recording tag with depletion-target. The beads are washed three times with 150 μL of PBST. As a result, the 3′ end of recording tags with depletion-target are blocked, resulting in unextendable ends, and other recording tags keep extendable spacer A.

After washing three times with 150 μL of PBST and 150 μL of PBS, the beads are resuspended in 200 μL of 10 mM ammonium bicarbonate, pH 9, and 2 μL of 5 mM TCEP is added and incubated at 37° C. for 30 minutes for reduction. The iodoacetamide is added to the beads mixture to adjust to 20 mM and incubated at 37° C. for 30 minutes for cysteine alkylation. After washing three times with 150 μL of PBST, the beads are incubated with 150 μL of acetylation reaction solution including 50 mM Ac-NHS, 50 mM carbonate/bicarbonate, 4 M Urea at 60° C. for 60 minutes. The trypsin is added to the beads mixture with 150 μL of Tris-HCl, pH 8.0 to adjust to 0.4 mg/mL and incubated at 37° C. overnight for proteins/peptides digestion. The trypsinized beads are washed three times with 150 μL of PBST for protein sequencing.

For protein sequencing, N-terminus specific binding agents are used to identify the amino acids at N-terminus of peptides on beads. The trypsinized peptides immobilized recording tag beads are incubated with 150 μL of 50 nM binding agents conjugated to coding tag containing and complementary sequences to spacer A (SP_A′) and spacer C (SP_C′) in PBST at 25° C. for 15 minutes. Unbound binding agent-coding tag conjugates are washed twice with 150 μL of PBST+500 mM NaCl, and then incubated with the 150 μL of encoding mixture containing 0.125 U/μL Klenow fragment (3′->5′ exo-), dNTP mixture (125 UM for each), 50 mM Tris-HCl (pH, 7.5), 2 mM MgSO₄, 50 mM NaCl, 1 mM DTT, 0.1% Tween-20, 0.1 mg/mL BSA and cycle capping oligo SpA_SpC at 25° C. for 5 minutes to copy the binding agent barcode and the spacer C to the recording tag containing extendable spacer A at the 3′ end. The beads are washed three times with 150 μL of PBST. As a result, recording tags other than depletion-target are installed with spacer C for additional protein sequencing cycles.

After multicycle steps for protein sequencing, the recording tags containing protein sequence information are capped by NGS adaptor sequences, followed by PCR for next generation sequencing.

### Example 11. Modification by Restriction Enzyme Digestion

Recording tag containing 5′ phosphorylated, DBCO modification and BamHI sequence at internal position, and spacer A (SP_A) sequence at 3′ end is hybridized to hairpin DNA immobilized on Sepharose™ beads by incubation at 37° C. for 30 min, washed once with PBST, and ligated by incubation with 150 μL of ligation mixture containing 66 mM Tris-HCl, 10 mM MgCl₂, 1 mM DTT, 1 mM ATP, 7.5% PEG 6000, 1 L of Quick ligase (NEB) at 25° C. for 30 min. The DBCO-modified recording tag beads are washed three times with 150 μL of PBST (PBS+0.1% Tween-20). On the other hand, proteins/peptides mixture in biological samples (e.g., plasma, cells, tissues) are incubated with azide-PEG₄-NHS ester, and the azide-modified protein/peptides are purified using 7K MWCO Zeba Spin Desalting Columns (Thermo Fisher). The azide-modified protein/peptides mixture is incubated with DBCO-modified recording tag beads at 37° C. overnight to immobilize protein/peptide on recording tag beads. The proteins/peptides immobilized recording tag beads are washed three times with 150 μL PBST for assay.

First cycle binding agents (e.g., antibody, chemicals, proteins) are used to block depletion-target proteins and peptides. The proteins/peptides immobilized recording tag beads containing a recording tag with spacer A (SP_A) at the 3′ end are incubated with 150 μL of 50 nM binding agents conjugated to oligo containing 8nt complementary sequences to BamHI site in recoding tags in PBST at 25° C. for 30 minutes. Unbound binding agent-coding tag conjugates are washed twice with 150 μL of PBST+500 mM NaCl, and then incubated with the 150 μL of restriction enzyme mixture containing 50 mM Potassium Acetate, 20 mM Tris-acetate, 100 μg/mL Recombinant Albumin and 1 U BamHI (NEB) at 37° C. for 30 minutes to digest recording tags with depletion-target. The beads are washed three times with 150 μL of PBST. As a result, the recording tags with depletion-target are digested, resulting in an unreadable sequence, and other recording tags keep extendable spacer A.

After washing three times with 150 μL of PBST and 150 μL of PBS, the beads are resuspended in 200 μL of 10 mM ammonium bicarbonate, pH 9, and 2 μL of 5 mM TCEP is added and incubated at 37° C. for 30 minutes for reduction. The iodoacetamide is added to the beads mixture to adjust to 20 mM and incubated at 37° C. for 30 minutes for cysteine alkylation. After washing three times with 150 μL of PBST, the beads are incubated with 150 μL of acetylation reaction solution including 50 mM Ac-NHS, 50 mM carbonate/bicarbonate, 4 M Urea at 60° C. for 60 minutes. The trypsin is added to the beads mixture with 150 μL of Tris-HCl, pH 8.0 to adjust to 0.4 mg/mL and incubated at 37° C. overnight for proteins/peptides digestion. The trypsinized beads are washed three times with 150 μL of PBST for protein sequencing.

For protein sequencing, N-terminus specific binding agents are used to identify the amino acids at N-terminus of peptides on beads. The trypsinized peptides immobilized recording tag beads are incubated with 150 μL of 50 nM binding agents conjugated to coding tag containing and complementary sequences to spacer A (SP_A′) and spacer C (SP_C′) in PBST at 25° C. for 15 minutes. Unbound binding agent-coding tag conjugates are washed twice with 150 μL of PBST+500 mM NaCl, and then incubated with the 150 μL of encoding mixture containing 0.125 U/μL Klenow fragment (3′->5′ exo-), dNTP mixture (125 UM for each), 50 mM Tris-HCl (pH, 7.5), 2 mM MgSO₄, 50 mM NaCl, 1 mM DTT, 0.1% Tween-20, 0.1 mg/mL BSA and cycle capping oligo SpA_SpC at 25° C. for 5 minutes to copy the binding agent barcode and the spacer C to the recording tag containing extendable spacer A at the 3′ end. The beads are washed three times with 150 μL of PBST. As a result, recording tags other than depletion-target are installed with spacer C for additional protein sequencing cycles.

After multicycle steps for protein sequencing, the recording tags containing protein sequence information are capped by NGS adaptor sequences, followed by PCR for next generation sequencing.

### Example 12. Modification by Chemical Ligation

Recording tag containing 5′ phosphorylated, DBCO modification at internal position and azide modified spacer A (SP_A) sequence at 3′ end is hybridized to hairpin DNA immobilized on Sepharose™ beads by incubation at 37° C. for 30 min, washed once with PBST, and ligated by incubation with 150 μL of ligation mixture containing 66 mM Tris-HCl, 10 mM MgCl₂, 1 mM DTT, 1 mM ATP, 7.5% PEG 6000, 1 μL of Quick ligase (NEB) at 25° C. for 30 min. The DBCO-modified recording tag beads are washed three times with 150 μL of PBST (PBS+0.1% Tween-20). On the other hand, proteins/peptides mixture in biological samples (e.g., plasma, cells, tissues) are incubated with azide-PEG₄-NHS ester, and the azide-modified protein/peptides are purified using 7K MWCO Zeba Spin Desalting Columns (Thermo Fisher). The azide-modified protein/peptides mixture is incubated with DBCO-modified recording tag beads at 37° C. overnight to immobilize protein/peptide on recording tag beads. The proteins/peptides immobilized recording tag beads are washed three times with 150 μL PBST for assay.

First cycle binding agents (e.g., antibody, chemicals, proteins) are used to block depletion-target proteins and peptides. The proteins/peptides immobilized recording tag beads containing a recording tag with spacer A (SP_A) at the 3′ end are incubated with 150 μL of 50 nM binding agents conjugated to coding tag containing alkyne-modified sequence complementary to spacer A in PBST at 25° C. for 30 minutes to hybridize to spacer A. Unbound binding agent-coding tag conjugates are washed twice with 150 μL of PBST+500 mM NaCl, and then incubated with the 150 μL of click ligation mixture containing 100 mM HEPES (pH 7.5), 4 mM CuSO₄, 8 mM THPTA, 20 mM Sodium Ascorbate at 25° C. for 1 hour to block the spacer A of recording tag with depletion-target. The beads are washed three times with 150 μL of PBST. As a result, the 3′ spacer A of recording tags with depletion-target are blocked, resulting in unextendable ends, and other recording tags keep extendable spacer A.

After washing three times with 150 μL of PBST and 150 μL of PBS, the beads are resuspended in 200 μL of 10 mM ammonium bicarbonate, pH 9, and 2 μL of 5 mM TCEP is added and incubated at 37° C. for 30 minutes for reduction. The iodoacetamide is added to the beads mixture to adjust to 20 mM and incubated at 37° C. for 30 minutes for cysteine alkylation. After washing three times with 150 μL of PBST, the beads are incubated with 150 μL of acetylation reaction solution including 50 mM Ac-NHS, 50 mM carbonate/bicarbonate, 4 M Urea at 60° C. for 60 minutes. The trypsin is added to the beads mixture with 150 μL of Tris-HCl, pH 8.0 to adjust to 0.4 mg/mL and incubated at 37° C. overnight for proteins/peptides digestion. The trypsinized beads are washed three times with 150 μL of PBST for protein sequencing.

For protein sequencing, N-terminus specific binding agents are used to identify the amino acids at N-terminus of peptides on beads. The trypsinized peptides immobilized recording tag beads are incubated with 150 μL of 50 nM binding agents conjugated to coding tag containing and complementary sequences to spacer A (SP_A′) and spacer C (SP_C′) in PBST at 25° C. for 15 minutes. Unbound binding agent-coding tag conjugates are washed twice with 150 μL of PBST+500 mM NaCl, and then incubated with the 150 μL of encoding mixture containing 0.125 U/μL Klenow fragment (3′->5′ exo-), dNTP mixture (125 μM for each), 50 mM Tris-HCl (pH, 7.5), 2 mM MgSO₄, 50 mM NaCl, 1 mM DTT, 0.1% Tween-20, 0.1 mg/mL BSA and cycle capping oligo SpA_SpC at 25° C. for 5 minutes to copy the binding agent barcode and the spacer C to the recording tag containing hybridizable spacer A at the 3′ end. The beads are washed three times with 150 μL of PBST. As a result, recording tags other than depletion-target are installed with spacer C for additional protein sequencing cycles.

After multicycle steps for protein sequencing, the recording tags containing protein sequence information are capped by NGS adaptor sequences, followed by PCR for next generation sequencing.

### Example 13. Competitive Attenuation of Signal Generated from Abundant Polypeptides During the Encoding Assay

Polypeptides isolated from biological samples (e.g., plasma, cells, tissues) are chemically linked to hairpin capture DNA immobilized on beads (see Example 2). Each capture DNA contains a recording tag with spacer A (SP_A) at 3′ end. A plurality of polypeptides is immobilized and an exemplary immobilized polypeptide (protein A) which encoding signal needs to be adjusted before NGPS assay is shown on FIG. 7A. Prior to NGPS assay, protein A is contacted with a mixture of binding agents comprising at least two binding agents that can specifically bind to protein A (cognate binding agents) and each conjugated with coding tag comprising either Sp_B′ (first binding agent, representing 1% of the mixture) or Sp_X′ (second binding agent, representing 99% of the mixture) spacer sequences. Following binding of the binding agents to protein A, the recording tags associated with Protein A are extended (modified) to contain either Sp_B or Sp_X sequence (FIG. 7A). Next, the plurality of proteins is digested with a trypsin and peptides remaining immobilized on the solid support are subjected to the NGPS peptide sequencing assay. Peptide A remains immobilized after digestion of Protein A with trypsin.

During the NGPS peptide sequencing assay, peptide A is contacted with a plurality of binding agents specific for different NTAA residues (see FIG. 2). Each binding agent comprises a coding tag having a Sp_B′ spacer at the 3′ end and complementary to the Sp_B sequence present in the fraction of the recording tags associated with peptide A. Following binding of cognate binding agent to the NTAA of peptide A, the complementary spacers Sp_B′ and Sp_B form hybridization duplexes and the identifying information regarding the binding agent (present in the barcode Sp_C′) is transferred by primer extension from the coding tag of the binding agent to the recording tag extended with the Sp_B sequence, generating a further extended recording tag. Only protein A associated with the recording tag having the Sp_B sequence is encoded, while extension of the recording tag having the Sp_X sequence is blocked (FIG. 7B).

### Example 14. Capping of an Abundant Protein with an Antibody-Based Binder to Suppress or Block the Encoding Signal

This example is based on FIGS. 3A-3B. A solid support comprises porous beads (NHS-Activated Sepharose High Performance, Cytiva, USA), on which a variety of polypeptides from a human plasma sample is attached including an abundant protein human serum albumin (HSA). Immobilized polypeptides are each associated with a nucleic acid recording tag as disclosed in Example 2. Immobilized polypeptides are set for a high-throughput polypeptide analysis that comprises an encoding reaction (e.g., see FIG. 1C and FIG. 2); however, it is desirable to suppress an encoding signal for abundant analytes, such as HSA (reduce amounts of extended recording tags associated with abundant analytes that need to be sequenced).

The binder, anti-human serum albumin antibody (Southernbiotech, Catalog No. 2080-01) is conjugated to a nucleic acid coding tag (comprising sequence CTGGAGCAGAGGAGAAGCAGTGCTCCAGGC. SEQ ID NO: 13) comprising a barcode flanked by 2nt overhang (indicated as SP_A′ in FIG. 3A) and a Type IIS restriction enzyme BtsI-V2 binding sequence with 2 bp digestion site (indicated as SP_B′). The coding tag is attached to the antibody via methyltetrazine (mTet)/trans-cyclooctene (TCO) click chemistry (standard conditions). Beads with immobilized plasma proteins are incubated with the binder (125 nM anti-HSA antibody-coding tag conjugate) in 1× Quick Ligation Reaction Buffer (66 mM Tris-HCl, 10 mM MgCl₂, 1 mM Dithiothreitol, 1 mM ATP, 7.5% PEG6000, pH 7.6, New England Biolabs, USA) supplemented with 0.1% Tween-20 at 37° C. for 2 hours. After washing twice with PBS+0.1% Tween-20 at 37° C., the beads are incubated with 1× quick ligation buffer comprising 125 μM of each dNTP. 0.1% Tween-20, 12.5 U/μL T4 DNA ligase (New England Biolabs, USA), 0.125 U/μL Klenow fragment exo-(MClab, USA) and 0.05 U/μL BtsI-v2 (New England Biolabs, USA) for 15 min at 25° C. The beads are washed once with PBS+0.1% Tween-20, once with 0.1M NaOH+0.1% Tween-20, and twice with PBS+0.1% Tween-20. After the described encoding reaction (based on the architecture shown in FIG. 1C), the spacer SP_A of the recording tag associated with HSA (protein B. FIG. 3A) is extended with spacer SP_B transferred from the anti-HSA antibody-coding tag conjugate during the encoding reaction.

The polypeptide conjugates immobilized on beads are then treated as illustrated in FIG. 3B. In the first step, the polypeptide conjugates immobilized on beads are digested into peptides by incubating with 50 μL of Lysis Buffer (Easy Pep™ MS Sample Prep Kits, Thermo Fisher Scientific). 25 μL of reduction solution (EasyPep™ MS Sample Prep Kits, Thermo Fisher Scientific) are added and incubated at 37° C. for 15 min with shaking at 850 rpm. After that. 25 μL of alkylation solution (EasyPep™ MS Sample Prep Kits, Thermo Fisher Scientific) are added and incubated at 37° C. for 15 min with shaking at 850 rpm. After removing the liquid, freshly-prepared 10 mM Sulfo-NHS-Acetate (Pierce Catalog 26777) solution (50 mM HEPES buffer, pH 7.5) are added to the beads and incubated at 37° C. for 10 min. A solution of Tris-HCl pH7.5 is added to quench the reaction, and the final concentration of the Tris-HCl is 20 mM. After washing, 100 μl of 9.2 ng/μL Trypsin/LysC (EasyPep™ MS Sample Prep Kits, Thermo Fisher Scientific) in Lysis buffer is added to digest the immobilized polypeptide conjugates. The incubation is conducted at 37° C. for 3 h with stirring at 850 rpm. The beads are washed twice with 1 M NaCl, twice with PBS, twice with a mixture of acetonitrile:H₂O (1:1 ratio), and twice with PBS.

In the second step, NGPS reactions as illustrated in FIG. 2 are performed on immobilized peptides generated during the first step (protease digestion). An exemplary N-terminal modifier (NTM) M64 is installed on NTAA residues of immobilized peptides, providing higher affinity and specificity during binding reactions with metalloprotein-based binding agents that recognize NTM-modified NTAA residues (a proper size of NTM that fit a binding pocket of the binding metalloprotein binding agent is required, see also US 2022/0283175 A1, incorporated herein by reference), and also compatibility with removal of the NTM-modified NTAA residues after binding by a Cleavase enzyme (see U.S. Pat. No. 11,427,814, incorporated herein by reference).

Exemplary method of installing M64 NTM onto NTAA residues of immobilized peptides, shown as NTAA-PP. Beads with immobilized peptides are treated with 25 μL of 0.4 M MOPS buffer, pH=7.6 and 25 μL of acetonitrile (ACN). Separately, the active ester reagent is prepared from M64 and dissolved in 25 μL DMA and 25 μL ACN to a concentration of 0.05 M stock solution. Then, 50 μL of the active ester stock solution is added to the peptide-ACN: MOPS solution and incubated at 65° C. for 60 minutes. Upon completion, the peptides were functionalized with the respective modification as shown in the scheme below.

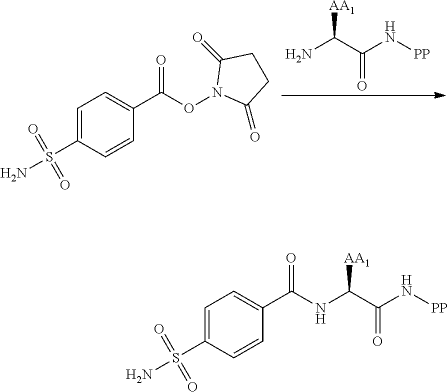

Next, the binding moiety having specificity towards M64-modified N-terminal F residues of immobilized peptides (engineered M64-F protein (SEQ ID NO: 14), see also US 2022/0283175 A1) is conjugated to a nucleic acid coding tag comprising the following sequence: CGTGAGCAGAGGAGAAGCAGTGCTCACGGC; SEQ ID NO: 15. which comprises a barcode flanked by 2nt overhang (indicated as SP_A′ in FIG. 3B) and a Type IIS restriction enzyme BtsI-V2 binding sequence with 2 bp digestion site (indicated as SP_C′ in FIG. 3B), generating a binding agent specific for modified N-terminal F residues. Similarly, binding agents specific for different M64-modified NTAA residues may also be generated (see US 2022/0283175 A1), wherein each binding agent comprises a unique coding tag that comprises identifying information regarding the binding agent. Such binding agents may be used as a mixture in the NGPS assay (see FIG. 2). The coding tag unique for each binding agent is attached to the binding moiety via a I-Sp18 PEG linker shown below:

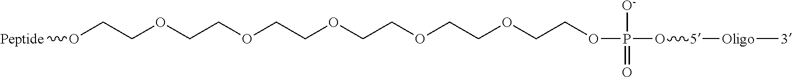

Peptide analysis illustrated in FIG. 3B is performed as follows. Beads with M64-N-terminally functionalized peptides are incubated with a mixture of binding agents containing 50 nM of each binding agent (protein-nucleic acid conjugate) in 50 mM MOPS buffer, 33 mM NaSO4, 1 mM EDTA and 0.1% Tween-20, pH 7.5 at 25° C. for 30 minutes. After washing twice with PBS+0.1% Tween-20, the beads are incubated with 1× quick ligation buffer, 125 μM dNTP each, 0.1% Tween-20, 12.5 U/μL T4 DNA ligase (New England Biolabs, USA), 0.125 U/μL Klenow fragment exo-(MClab, USA) and 0.05 U/μL BtsI-v2 (New England Biolabs, USA) for 15 min at 25° C. The beads are washed once with PBS+0.1% Tween-20, once with 0.1M NaOH+0.1% Tween-20, and twice with PBS+0.1% Tween-20. After this step, the region SP_A of the recording tag attached to the target peptide (peptide A, FIG. 3B) is extended by incorporating the region SP_C comprising identifying barcode for the binding agent that was bound to the modified NTAA of peptide A during this step. To insert universal priming site into the extended recording tag for NGS library preparation, the beads are incubated with 1× quick ligation buffer, 12.5 U/μL T4 DNA ligase, 0.125 U/μL Klenow fragment exo-, 125 uM dNTP each, 0.1% Tween-20, and 0.4 μM capping oligo for region SP_C at 25° C. for 15 minutes. After washing once with PBS+0.1% Tween-20, once with 0.1 M NaOH+0.1% Tween-20, and twice with PBS+0.1% Tween-20, the beads are incubated with 1× rCutSmart Buffer, 0.1% Tween-20, and 0.02 U/μL of USER enzyme (New England Biolabs, USA) at 37° C. for 30 minutes. The beads are washed once with PBS+0.1% Tween-20, once with 0.1 M NaOH+0.1% Tween-20, and twice PBS+0.1% Tween-20, and applied to polymerase reaction (PCR) for next-generation sequencing (NGS). Binding history of peptide A and other peptides that have appropriate spacer sequences in the associated extended recording tag is decoded from the NGS data, which in turn can be translated into peptide sequences.

### Example 15. Adjusting Encoding Signals for Several Targeted Analytes

This example is based on an embodiment shown in FIGS. 7A-7B. A solid support comprises porous beads (NHS-Activated Sepharose High Performance, Cytiva, USA), on which a variety of polypeptides from a human plasma sample is attached including an abundant protein A, as well as proteins B, C and D, which need to be detected by high-throughput peptide sequencing (e.g., NGPS, see FIG. 2). For this assay, it is desirable to suppress an encoding signal for abundant analytes, such as protein A (reduce amounts of extended recording tags associated with abundant analytes that need to be sequenced). Immobilized polypeptides are each associated with a nucleic acid recording tag as disclosed in Example 2.

The mixture of antibody-based binders is prepared comprising two binders for protein A, and a binder for each of proteins B, C and D. The first binder for protein A and all binders for proteins B, C and D comprise “functional” coding tags containing regions SP_A′ and SP_B′ (see FIG. 7A), whereas the second binder for protein A comprises “dead end” coding tag containing regions SP_A′ and SP_X′ (see FIG. 7A). The ratio of the first binder to the second binder in the mixture of binders is 1:99. Following binding and encoding (conditions are the same as described in Example 14), the recording tags associated with proteins B, C and D are extended with SP_B; however, only a small fraction (appr. 1/100) of the recording tags associated with protein A are extended with SP_B, whereas the remaining recording tags associated with protein A are extended with SP_X (see FIG. 7B).

Next, the proteins A, B, C and D immobilized on beads are digested into peptides as described in Example 14, and the resulting peptides A, B, C and D immobilized on beads are subjected to the NGPS assay using a mixture of binding agents specific to different M64-modified NTAA residues as described in Example 14. Only peptide-associated extended recording tags comprising the SP_B region are able to participate in information transfer from coding tags of binding agents, whereas information transfer to extended recording tags comprising the SP_X region is blocked (SP_X would not form a duplex with a coding tag of a binding agent, see FIG. 7B). Thus, encoding signal (amount of NGS data) from peptide A is reduced relative to encoding signal from peptides B, C and D by ˜100 fold.

### Example 16. Encoding Assays for Other Types of Macromolecules

The approaches described in Figures and the Examples shown above can be adopted for other types of macromolecules, such as lipid, carbohydrate or macrocycle. To perform encoding assay, such macromolecules need to be immobilized on a solid support (such as beads) and associated with nucleic acid recording tag. The encoding steps remain the same regardless of the type of the macromolecule immobilized. The association with the recording tag can be direct (such as covalent attachment) or indirect (such as association through a solid support). In the latter case, the recording tag should co-localize or be in a close proximity with the macromolecule during the encoding assay. Binding agents can be chosen to bind specifically to a component of the macromolecule. Each binding agent needs to be conjugated to corresponding nucleic acid coding tag that contains a barcode with identifying information regarding the binding agent. During encoding, the barcode information is transferred to the recording tag associated with the macromolecule, generating the extended recording tag, so that binding history of the macromolecule is recorded into the extended recording tag. The binding cycle can be repeated multiple times using different binding agents interacting with the macromolecule, either separately, or in a mixture. Below, representative methods known in the art are disclosed that can be utilized for adaptation of the disclosed encoding assay for macromolecules of different types, such as a carbohydrate, a lipid or a macrocycle.

First, exemplary binding agents that can specifically bind to components of a carbohydrate, a lipid or a macrocycle are known. For example, lectins are carbohydrate-binding proteins that can selectively recognize glycan epitopes of free carbohydrates or glycoproteins, and can be utilized as specific binding agents for macromolecules that contain carbohydrates. Importantly, there are known lectins that recognize different components of carbohydrates, such as mannose-binding lectins, galactose/N-acetyl glucosamine-binding lectins, sialic acid/N-acetyl glucosamine-binding lectins, fucose-binding lectins (disclosed for example, in WO 2012/049285 A1). Also, lipid-binding proteins are well-known and can be utilized as binding agents (see, for example, Bernlohr D A, et al., Intracellular lipid-binding proteins and their genes. Annu Rev Nutr. 1997; 17:277-303). Lipid-binding antibodies are commonly known and can be utilized as binding agents for macromolecules that contain lipids (see, for example, Alving C R. Antibodies to lipids and liposomes: immunology and safety. J Liposome Res. 2006; 16 (3): 157-66). Furthermore, proteins that specifically bind to macrocycles are also known (see, for example, Villar E A, et al., How proteins bind macrocycles. Nat Chem Biol. 2014 September; 10 (9): 723-31; Hunter™, et al., Protein recognition of macrocycles: binding of anti-HIV metallocyclams to lysozyme. Proc Natl Acad Sci USA. 2005 Feb. 15; 102 (7): 2288-92).

Second, an exemplary carbohydrate detection encoding assay can be performed as follows, utilizing methods known in the art.

Approach I. Reductive amination (based on Yang S J, Zhang H. Glycan analysis by reversible reaction to hydrazide beads and mass spectrometry. Anal Chem. 2012; 84 (5): 2232-2238).

(a) Generate an immobilized recording tag-attached carbohydrate conjugate. Oxidize carbohydrates with sodium periodate to generate an aldehyde. Conjugate amine terminated DNA recording tag and reduce the resulting imine using sodium cyanoborohydride to generate a carbohydrate-recording tag conjugate. Preferably, hydrazide, alkoxyamine, or similarly reactive DNA may be employed to generate more stable reaction products (e.g., hydrazones) that do not require reducing agents. Immobilize DNA-coupled carbohydrate to a solid support via the DNA recording tag as described in Example 2.

(b) Generate lectin-DNA coding tag (the binding agent-coding tag) conjugates by utilizing SpyCatcher-concanavalin A (ConA) fusion as described earlier. Coding tag contains a barcode with identifying information regarding ConA.

(c) Transfer barcode information from lectin-associated coding tag to the recording tag as described in Example 14, thus analyzing whether the carbohydrate contains a component that binds to ConA.

Approach II. Diazo coupling (based on Matsuura K, et al., Facile synthesis of stable and lectin-recognizable DNA-carbohydrate conjugates via diazo coupling. Bioconjug Chem. 2000 Mar.-April; 11 (2): 202-11). In approach II, step (a) (immobilization of recording tag-attached carbohydrate conjugate) can be performed as follows. 1) Aminate carbohydrate with ammonium hydrogen carbonate in water to generate β-glycosylamines; 2) Convert amine to amide with carboxylate derivatives bearing a nitrophenyl functionality. Hydrogenate nitro groups over palladium catalyst and treat with NaNO₂ and HCl to provide the diazo compounds.

Steps (b) and (c) are the same as in the in approach I.

Third, an exemplary lipid detection encoding assay can be performed as follows, utilizing methods known in the art.

Approach I. Fatty acids (based on Hiroshi Miwa, High-performance liquid chromatographic determination of free fatty acids and esterified fatty acids in biological materials as their 2-nitrophenylhydrazides, Analytica Chimica Acta, Volume 465, Issues 1-2, 2002, Pages 237-255, ISSN 0003-2670).

(a) Extract fatty acids from a biological source and activate carboxylic acid with EDC/CDI chemistry. Couple amine- or hydrazide-terminated DNA recording tag to generate a recording tag-attached lipid conjugate. Immobilize DNA-coupled lipid to a solid support via the DNA recording tag as described in Example 2.

Approach II. Reactive lipids (based on X. Wei & H. Yin (2015) Covalent modification of DNA by α, β-unsaturated aldehydes derived from lipid peroxidation: Recent progress and challenges, Free Radical Research, 49:7, 905-917).

(a) Obtain a reactive lipid substrate such as malondialdehyde (MDA) or 4-hydroxynonenal (HNE); couple hydrazide-terminated DNA recording tag to reactive lipid species. Alternatively, couple amine-terminated DNA recording tag to aldehyde on reactive lipid and reduce resulting imine with sodium cyanoborohydride. In the next step for both approaches, generate a binding agent-DNA coding tag conjugate by utilizing SpyCatcher-binding agent fusion as described earlier. Coding tag contains a barcode with identifying information regarding the binding agent. Fatty acid-binding protein (FABP), other lipid binding proteins or lipid binding antibodies can be used as a binding agent. Finally, transfer barcode information from binding agent-associated coding tag to the recording tag as described in Example 14, thus analyzing whether the lipid contains a component that binds to the binding agent.

Forth, an exemplary macrocycle (microcystin) detection encoding assay can be performed as follows, utilizing methods known in the art, based on McElhiney J. et al., Rapid isolation of a single-chain antibody against the cyanobacterial toxin microcystin-LR by phage display and its use in the immunoaffinity concentration of microcystins from water. Appl Environ Microbiol. 2002 November; 68 (11): 5288-95.

(a) Generate DNA recording tag-coupled microcystin by reacting dehydroalanine of microcystin with 2-mercaptoethylamine to generate a primary amine, followed by coupling DNA recording tag to primary amine using an amine reactive DNA recording tag (e.g., NHS-DNA derivative).

(b) Generate single chain antibody-SpyCatcher binding agent that recognizes microcystin. Single chain antibody production is described in McElhiney J. et al. 2002. Couple DNA coding tag to SpyTag (the coding tag contains a barcode with identifying information regarding the single chain antibody), followed by reacting with SpyCatcher to generate the binding agent-coding tag conjugate as described earlier.

(c) Transfer barcode information from single chain antibody-associated coding tag to the recording tag as described in Example 14, thus analyzing whether the macromolecule contains microcystin.

The present disclosure is not intended to be limited in scope to the particular disclosed embodiments, which are provided, for example, to illustrate various aspects of the disclosure. Various modifications to the compositions and methods described will become apparent from the description and teachings herein. Such variations may be practiced without departing from the true scope and spirit of the disclosure and are intended to fall within the scope of the present disclosure. These and other changes can be made to the embodiments in light of the above-detailed description. In general, in the following claims, the terms used should not be construed to limit the claims to the specific embodiments disclosed in the specification and the claims, but should be construed to include all possible embodiments along with the full scope of equivalents to which such claims are entitled. Accordingly, the claims are not limited by the disclosure.

1. A method for analyzing a plurality of different polypeptides immobilized on a support, the method comprising:
(a) contacting the plurality of different polypeptides comprising a first polypeptide and a second polypeptide with a binder, wherein the first polypeptide is associated with a first recording tag and the second polypeptide is associated with a second recording tag, and wherein the binder comprises: (i) a binding moiety capable of binding to the first polypeptide; and (ii) a handle attached to the binding moiety and configured to bind to or react with the first recording tag;
(b) allowing the handle to bind to or react with the first recording tag brought in proximity by binding of the binder to the first polypeptide, thereby modifying the first recording tag to generate a modified first recording tag associated with the first polypeptide;
(c) optionally, fragmenting the plurality of different polypeptides immobilized on the support to generate fragments of different polypeptides immobilized on the support;
(d) contacting the plurality of different polypeptides or the fragments of different polypeptides with a plurality of binding agents, wherein each binding agent comprises: (i) a binding moiety capable of binding to a portion or component of a polypeptide of the plurality of different polypeptides or the fragments thereof; and (ii) a coding tag that comprises identifying information regarding the binding agent;
(e) allowing transfer of identifying information from coding tags of the plurality of binding agents to recording tags associated with the plurality of different polypeptides or the fragments of different polypeptides, thereby generating an extended second recording tag associated with the second polypeptide or fragment thereof upon binding of a binding agent to the second polypeptide or fragment thereof, wherein transfer of identifying information to the modified first recording tag associated with the first polypeptide or fragment thereof is suppressed or blocked; and
(f) analyzing the extended second recording tag to obtain identifying information regarding the binding agent that binds to the second polypeptide or fragment thereof, thereby obtaining information about the second polypeptide or fragment thereof, wherein the analyzing comprises nucleic acid sequencing.

2. The method of claim 1, wherein the handle comprises a polynucleotide and binds to the first recording tag via nucleic acid hybridization.

3. (canceled)

4. The method of claim 1, wherein the handle does not comprise a polynucleotide.

5. The method of claim 1, wherein the handle comprises a protein, wherein the protein is an enzyme.

6. The method of claim 5, wherein the protein is a nuclease that is configured, upon binding to the first recording tag, to produce an unfunctional first recording tag associated with the first polypeptide.

7. The method of claim 1, wherein upon binding to the first recording tag, the handle modifies the first recording tag to produce an unfunctional first recording tag associated with the first polypeptide.

8. The method of claim 7, wherein following binding of the handle to the first recording tag, the handle remains attached to the first recording tag to form an unfunctional first recording tag associated with the first polypeptide.

9. The method of claim 7, wherein the handle comprises a chemical moiety capable of reacting with the first recording tag to produce an unfunctional first recording tag associated with the first polypeptide.

10. The method of claim 1, wherein the plurality of binding agents comprises:
i) a first binding agent comprising a first coding tag that comprises identifying information regarding the first binding agent, wherein the first binding agent is capable of binding to the first polypeptide or fragment thereof, or a component thereof; and
ii) a second binding agent comprising a second coding tag that comprises identifying information regarding the second binding agent, wherein the second binding agent is capable of binding to the second polypeptide or fragment thereof, or a component thereof.

11-12. (canceled)

13. The method of claim 10, wherein the transfer of identifying information regarding the second binding agent from the second coding tag to the second recording tag comprises:
(o) generating a double-stranded nucleic acid comprising the second recording tag by (i) joining an end of the second recording tag to an end of the second coding tag, and (ii) optionally, extending the second recording tag using the second coding tag as a template by a polymerase; and
(p) cleaving the double-stranded nucleic acid to generate the extended second recording tag.

14-18. (canceled)

19. The method of claim 1, wherein (d) and (e) are repeated one or more times in sequential cycles.

20-21. (canceled)

22. The method of claim 19, further comprising removing a portion of the second polypeptide or fragment thereof prior to one or more of the sequential cycles of repeating (d) and (e).

23-26. (canceled)

27. The method of claim 1, wherein the first polypeptide and the second polypeptide are immobilized on the same support.

28-29. (canceled)

30. The method of claim 1, wherein the first and second polypeptides are of different amino acid sequences, and wherein on the support, molecules of the first polypeptide are more abundant than molecules of the second polypeptide.

31. The method of claim 1, wherein the first and second polypeptides are of the same amino acid sequences, and wherein on the support, molecules of the first polypeptide comprising a first post-translational modification are more abundant than molecules of the second polypeptide comprising a second post-translational modification.

32-34. (canceled)

35. A method for analyzing molecules of a polypeptide immobilized on a support, the method comprising:
(a) contacting the molecules of the polypeptide with a first binding agent and a second binding agent,
wherein each molecule of the polypeptide is associated with a recording tag immobilized on a support,
wherein the first binding agent comprises (i) a first binding moiety capable of binding to the polypeptide; and (ii) a first coding tag attached to the first binding moiety and comprising identifying information regarding the first binding agent, and
wherein the second binding agent comprises (i) a second binding moiety capable of binding to the polypeptide; and, optionally, (ii) a handle attached to the second binding moiety and configured to bind to or react with the recording tag;
(b) allowing transfer of the identifying information regarding the first binding agent from the first coding tag to the recording tag by primer extension and/or ligation to generate an extended recording tag, and optionally, allowing the handle to bind to or react with the first recording tag to generate a modified first recording tag;
(c) contacting the molecules of the polypeptide with a third binding agent comprising (i) a third binding moiety capable of binding to the polypeptide; and (ii) a third coding tag attached to the third binding moiety and comprising identifying information regarding the third binding agent;
(d) allowing transfer of the identifying information regarding the third binding agent from the third coding tag to the extended recording tag by primer extension and/or ligation to generate a further extended recording tag, wherein transfer of the identifying information regarding the third binding agent from the third coding tag to the recording tag or to the modified first recording tag is suppressed or blocked; and
(e) analyzing the further extended recording tag to obtain identifying information regarding the first binding agent and/or the third binding agent, thereby obtaining information about the polypeptide, wherein the analyzing comprises nucleic acid sequencing.

36. The method of claim 35, wherein the polypeptide is a first polypeptide, the method further comprising:
performing (a)-(e) for molecules of a second polypeptide different from the first polypeptide, wherein (i) corresponding first, second and third binding agents for the second polypeptide comprise binding moieties capable of binding to the second polypeptide; and (ii) a ratio between amounts of the first binding agent and the second binding agent is different for the first and the second polypeptides.

37. The method of claim 36, wherein molecules of the first polypeptide are more abundant than molecules of the second polypeptide, and the ratio between amounts of the first binding agent and the second binding agent for the first polypeptide is smaller than the ratio for the second polypeptide.

38. The method of claim 36 or 37, wherein the ratio for the first polypeptide and/or the ratio for the second polypeptide is selected or determined before contacting the molecules of the first and/or second polypeptides with the corresponding first and second binding agents.

39. The method of claim 38, comprising estimating relative abundance of the first and the second polypeptides in a biological sample, wherein the relative abundance correlates with the relative abundance of the first and the second polypeptides immobilized on supports.

40. The method of claim 35, wherein for the first polypeptide and/or for the second polypeptide, the binding moiety of the first binding agent is essentially identical to the binding moiety of the second binding agent.

41. The method of claim 36, wherein the first polypeptide and second polypeptide are immobilized on the same support.


SEQUENCE LISTING (20 sequences)

SEQ ID NO: 1 - DNA, 20 bp, synthetic construct
  source 1..20: mol_type = other DNA
  misc_feature 1..20: note = P5 primer
aatgatacggcgaccaccga

SEQ ID NO: 2 - DNA, 24 bp, synthetic construct
  source 1..24: mol_type = other DNA
  misc_feature 1..24: note = P7 primer
caagcagaagacggcatacgagat

SEQ ID NO: 3 - protein, 19 aa, synthetic construct
  REGION 1..19: note = FA-terminal peptide
FAGVAMPGAEDDVVGSGSK

SEQ ID NO: 4 - protein, 20 aa, synthetic construct
  REGION 1..20: note = AFA-terminal peptide
AFAGVAMPGAEDDVVGSGSK

SEQ ID NO: 5 - protein, 19 aa, synthetic construct
  REGION 1..19: note = AA-terminal peptide
AAGVAMPGAEDDVVGSGSK

SEQ ID NO: 6 - DNA, 34 bp, synthetic construct
  source 1..34: mol_type = other DNA
  misc_feature 1..34: note = DBCO-DNA
  modified_base 1: mod_base = OTHER; note = 5'-phosphorylated
  modified_base 22: mod_base = OTHER; note = modified with dibenzocyclooctyne (DBCO)
caagttctcagtaatgcgtagtccgcgacactag

SEQ ID NO: 7 - protein, 19 aa, synthetic construct
  REGION 1..19: note = Test peptide
FSGVAMPGAEDDVVGSGSK

SEQ ID NO: 8 - protein, 20 aa, synthetic construct
  REGION 1..20: note = Test peptide
AFSGVAMPGAEDDVVGSGSK

SEQ ID NO: 9 - DNA, 22 bp, synthetic construct
  source 1..22: mol_type = other DNA
  misc_feature 1..22: note = Recording tag
  misc_difference 8..10: note = n is a, c, g, or t
  modified_base 1: mod_base = OTHER; note = conjugated to another oligonucleotide through internal amino modifier C6 dT
ccgatctnnnttgtcacactac

SEQ ID NO: 10 - DNA, 22 bp, synthetic construct
  source 1..22: mol_type = other DNA
  misc_feature 1..22: note = Recording tag
  misc_difference 8..10: note = n is a, c, g, or t
  modified_base 1: mod_base = OTHER; note = conjugated to another oligonucleotide through internal amino modifier C6 dT
ccgatctnnnaggacacactac

SEQ ID NO: 11 - DNA, 97 bp, synthetic construct
  source 1..97: mol_type = other DNA
  misc_feature 1..97: note = Capture DNA
  misc_difference 17..26: note = n is a, c, g, or t
cactcagtccattaacnnnnnnnnnnctagtgtcgcggactacgcattactgagaagcttgctagtcgacgtggtccttttggaccacgtcgactag

SEQ ID NO: 12 - DNA, 90 bp, synthetic construct
  source 1..90: mol_type = other DNA
  misc_feature 1..90: note = Capping oligo
  modified_base 90: mod_base = OTHER; note = modified with C3 (three carbon) spacer
gaagagtaattagatcggaagagcacacgtctgaactcgactggagttcagacgtgtgctcttccgatctaattactcttctagagatgg

SEQ ID NO: 13 - DNA, 30 bp, synthetic construct
  source 1..30: mol_type = other DNA
  misc_feature 1..30: note = Coding tag
ctggagcagaggagaagcagtgctccaggc

SEQ ID NO: 14 - protein, 256 aa, synthetic construct
  REGION 1..256: note = Engineered M64-F-binder
HWGYGKHNGPEHWHKDFPIAKGERQSPVDIDTHTAKYDPSLKPLSVSYDQATSLRILNNGHAFEVEFDDSQDKAVLKGGPLDGTYRLVQFHFHWGSLDGQGSEHTVDKKKYAAELHLVHWNTKYGDYGKAMQQPDGLAVLGIFLKVGSAKPGLQKVVDVLDSIKTKGKSADFTNFDPRGLLPESLDYWTYPGSGTVPPLLESVTWIVLKEPISVSSEQVLKFRKLNFNGEGEPEELMVDNWRPAQPLKNRQIKASF

SEQ ID NO: 15 - DNA, 30 bp, synthetic construct
  source 1..30: mol_type = other DNA
  misc_feature 1..30: note = Coding tag
cgtgagcagaggagaagcagtgctcacggc

SEQ ID NO: 16 - protein, 256 aa, synthetic construct
  source 1..256: note = Engineered M64-H-binder
  REGION 1..256
HWGYGKHNGPEHWHKDFPIAKGERQSPVDIDTHTAKYDPSLKPLSVSYDQATSLRILNDGHAFEVEFDDSQDKAVLKGGPLDGTYRLIQFHFHWGSLDGQGSEHTVDKKKYAAELHLVHWNTKYGDFGKALQQPDGMAILGIFLKVGSAKPGLQKVVDVLDSIKTKGKSADFTNFDPRGLLPESLDYWTYPGSQTIPPLLESVTWIVLKEPISVSSEQVLKFRKLNFNGEGEPEELMVDNWRPAQPLKNRQIKASF

SEQ ID NO: 17 - protein, 256 aa, synthetic construct
  REGION 1..256: note = Engineered M64-E-binder
HWGYGKHNGPEHWHKDFPIAKGERQSPVDIDTHTAKYDPSLKPLSVSYDQATSLRILNNGHAFLVEFDDSQDKAVLKGGPLDGTYRLKQFHFHWGSLDGQGSEHTVDKKKYAAELHLVHWNTKYGDYGKALQQPDGIAVLGIFLKVGSAKPGLQKVVDVLDSIKTKGKSADFTNFDPRGLLPESLDYWTYPGSRTIPPLLESVTWIVLKEPISVSSEQVLKFRKLNFNGEGEPEELMVDNWRPAQPLKNRQIKASF

SEQ ID NO: 18 - protein, 256 aa, synthetic construct
  REGION 1..256: note = Engineered M64-T-binder
HWGYGKHNGPEHWHKDFPIAKGERQSPVDIDTHTAKYDPSLKPLSVSYDQATSLRILNDGHAFRVEFDDSQDKAVLKGGPLDGTYRLKQFHFHWGSLDGQGSEHTVDKKKYAAELHLVHWNTKYGDFGKAGQQPDGIAILGIFLKVGSAKPGLQKVVDVLDSIKTKGKSADFTNFDPRGLLPESLDYWTYPGSETIPPLLESVTWIVLKEPISVSSEQVLKFRKLNFNGEGEPEELMVDNWRPAQPLKNRQIKASF

SEQ ID NO: 19 - protein, 723 aa, Thermomonas sp.
  REGION 1..723: note = Dipeptidyl peptidase from S46 family
MHKTRLVAALAAALATLAPAAWADEGMWVPQQLPEIAGALKKAGLKLDPKQLSDLTGDPMGAVVSLGGCTGSFVSPQGLVATNHHCAYGAIQLNSTPEKNLIKDGFNAPTQADELSAGPNARIYVLEGITDVTAQAKAAMAAAGNDPVARANALEAFEKKITSDCEAEPGYRCRVYSFMGGITYRLFKNLEIKDVRLVYAPPSSVGKFGGDIDNWMWPRHTGDFSFYRAYVGKDGKPAPYSKDNVPYRPKHWLKIADTPLGEGDFVMVAGYPGRTDRYALVAEFENTQNWLYPAISKAYKDQIALVEAAAKDNPEIAVKYAAALAGWNNTSKNFDGQLEGFKRNDVLAIKRREEAAVLEWLRARGKAGTPALEAHAALVKLVADTARTQERDLVLGSFNRTGIIGVAVNLYRLAIERQKPDAEREPGYQQRDLPVIEGSLKQMERRYVPAMDRQLRAYWLDRYVALPAAQHVAAVDAWLGGSDKAAAEAALARLDQSRLGSLEERLKWFNADRAAFEASTDPAIQYAVAVMPTLLAMEQQAKTRYGVALEARPRYLQAVVDYKKSKGQAVYPDANSTLRITYGHVKGYTGLNGKVYTPFTTLEEVAAKETGVEPFDNPKALLEAVAAKRYAGLADARLGTVPVNFLADLDITGGNSGSPVLDANGRLVGLAFDGTLESVASNWVFDPVLTRMISVDQRYMRWIMQEVMPAPQLLEELGVPPRQ

SEQ ID NO: 20 - protein, 700 aa, synthetic construct
  REGION 1..700: note = engineered dipeptidyl peptidase clone
DEGMWVPQQLPEIAGALKKAGLKLDPKQLSDLTGDPMGAVVSLGGCTGSFVSPQGLVATNHHCAYGAIQLNSTPEKNLIKDGFNAPTQADELSAGPNARIYVLEGITDVTAQAKAAMAAAGNDPVARANALEAFEKKITSDCEAEPGYRCRVYSFMGGITYRLFKNLEIKDVRLVYAPPSSVGKFGGDIDMGMWPTHTGDFSFYRAYVGKDGKPAPYSKDNVPYRPKHWLKIADTPLGEGDFVMVAGYPGRTDRYALVAEFENTQNWLYPAISKAYKDQIALVEAAAKDNPEIAVKYAAALAGWNRTSKNFDGQLEGFKRNDVLAIKRREEAAVLEWLRARGKAGTPALEAHAALVKLVADTARTQERDLVLGSFNRTGIIGVAVNLYRLAIERQKPDAEREPGYQQRDLPVIEGSLKQMERRYVPAMDRQLRAYWLDRYVALPAAQHVAAVDAWLGGSDKAAAEAALARLDQSRLGSLEERLKWFNADRAAFEASTDPAIQYAVAVMPTLLAMEQQAKTRYGVALEARPRYLQAVVDYKKSKGQAVYPDANSTLRITYGHVKGYTGLNGKVYTPFTTLEEVAAKETGVEPFDNPKALLEAVAAKRYAGLADARLGTVPVNFLADLDITGGNSGSPVLDANGRLVGLAFAVTLESVASNWVFDPVLTRMISVDQRYMRWIMQEVMPAPQLLEELGVPPRQ